(19) United States
(12) Patent Application Publication
(10) Pub. No.: US 2015/0344426 A1
(43) Pub. Date: Dec. 3, 2015

(54) COMPOUNDS AND THERAPEUTIC USES THEREOF

(71) Applicants: J. Adam Willardsen, Draper, UT (US); Jeffrey W. Lockman, Princeton Junction, NJ (US); Brett R. Murphy, Collbran, CO (US); Weston R. Judd, Farmington, UT (US); Kraig M. Yager, Murray, UT (US)

(72) Inventors: J. Adam Willardsen, Draper, UT (US); Jeffrey W. Lockman, Princeton Junction, NJ (US); Brett R. Murphy, Collbran, CO (US); Weston R. Judd, Farmington, UT (US); Kraig M. Yager, Murray, UT (US)

(73) Assignee: ALZHEIMER'S INSTITUTE OF AMERICA, INC., Kansas City, KS (US)

(21) Appl. No.: 14/582,641

(22) Filed: Dec. 24, 2014

Related U.S. Application Data
(63) Continuation of application No. PCT/US2013/048274, filed on Jun. 27, 2013.
(60) Provisional application No. 61/665,297, filed on Jun. 27, 2012.

(51) Int. Cl. C07D 213/58; C07D 401/12; C07D 403/12

(52) U.S. Cl. CPC C07D 213/58; C07D 403/12; C07D 401/12

(57) ABSTRACT

The invention relates to compounds, pharmaceutical compositions and methods useful for treating cancer, systemic or chronic inflammation, rheumatoid arthritis, diabetes, obesity, T-cell mediated autoimmune disease, ischemia, and other complications associated with these diseases and disorders.

### RELATED APPLICATIONS

This application is a continuation of International Patent Application No. PCT/US2013/048274, filed on Jun. 27, 2013, and titled “Compounds and Therapeutic Uses Thereof,” which claims priority to U.S. Provisional Application No. 61/665,297 filed on Jun. 27, 2012 and titled “Compounds and Therapeutic Uses Thereof,” the contents of both of which are hereby incorporated by reference in their entirety.

### FIELD OF THE INVENTION

The present invention relates generally to the field of medicinal chemistry. Specifically, the present invention provides compounds that inhibit Nicotinamide phosphoribosyltransferase (Nampt). The invention also provides methods for making these compounds, pharmaceutical compositions comprising these compounds, and methods for treating diseases with these compounds; particularly cancer, systemic or chronic inflammation, rheumatoid arthritis, diabetes, obesity, T-cell mediated autoimmune disease, ischemia, and other complications associated with these diseases and disorders, that respond favorably to the inhibition of Nampt.

### BACKGROUND OF THE INVENTION

Nicotinamide phosphoribosyltransferase (Nampt; also known as visfatin and pre-B-cell colony-enhancing factor 1 (PBEF)) catalyzes the condensation of nicotinamide (NaM) with 5-phosphoribosyl-1-pyrophosphate to yield nicotinamide mononucleotide. This is the first and rate-limiting step in one biosynthetic pathway that cells use to make nicotinamide adenine dinucleotide (NAD⁺).

NAD⁺ has many important cellular functions. Classically, it plays a role as a key coenzyme in metabolic pathways, where it continually cycles between its oxidized form (NAD⁺) and its reduced form (NADH). More recently, NAD⁺ has been shown to be involved in genome integrity maintenance, stress response, and Ca²⁺ signaling, where it is consumed by enzymes including poly(ADP-ribose) polymerases (PARPs), sirtuins, and cADP-ribose synthases, respectively. (Reviewed in Belenky, P. et al., NAD⁺ metabolism in health and disease. Trends Biochem. Sci. 32, 12-19 (2007).)

As a critical coenzyme in redox reactions, NAD⁺ is required in glycolysis and the citric acid cycle; where it accepts the high energy electrons produced and, as NADH, passes these electrons on to the electron transport chain. The NADH-mediated supply of high energy electrons is the driving force behind oxidative phosphorylation, the process by which the majority of ATP is generated in aerobic cells. Consequently, having sufficient levels of NAD⁺ available in the cell is critical for the maintenance of proper ATP levels in the cell. Understandably, reduction in cellular NAD⁺ levels by Nampt inhibition can be expected to eventually lead to depletion of ATP and, ultimately, cell death.

In view of the above, it is perhaps not surprising that inhibitors of Nampt are being developed as chemotherapeutic agents for the treatment of cancer. In fact, there are currently two Nampt inhibitors in clinical trials for the treatment of cancer (Holen, K. et al. The pharmacokinetics, toxicities, and biologic effects of FK866, a nicotinamide adenine dinucleotide biosynthesis inhibitor. Invest. New Drugs. 26, 45-51 (2008); Hovstadius, P. et al. A Phase I study of CHS 828 in patients with solid tumor malignancy. Clin. Cancer Res. 8, 2843-2850 (2002); Ravaud, A. et al., Phase I study and pharmacokinetic of CHS-828, a guanidino-containing compound, administered orally as a single dose every 3 weeks in solid tumours: an ECSG/EORTC study. Eur. J. Cancer. 41, 702-707 (2005); and von Heideman, A. et al. Safety and efficacy of NAD depleting cancer drugs: results of a phase I clinical trial of CHS 828 and overview of published data. Cancer Chemother. Pharmacol. (2009) September 30 [Epub ahead of print]).

Consequently, there is a clear need for compounds that inhibit Nampt, which can not only be used in the treatment of cancer, but can also be used in the treatment of systemic or chronic inflammation, rheumatoid arthritis, diabetes, obesity, T-cell mediated autoimmune disease, ischemia, and other complications associated with these diseases and disorders.

### BRIEF SUMMARY OF THE INVENTION

The present invention provides chemical compounds that inhibit the activity of Nampt. These compounds can be used in the treatment of cancer, systemic or chronic inflammation, rheumatoid arthritis, diabetes, obesity, T-cell mediated autoimmune disease, ischemia, and other complications associated with these diseases and disorders.

Specifically, the present invention provides compounds of Formula I

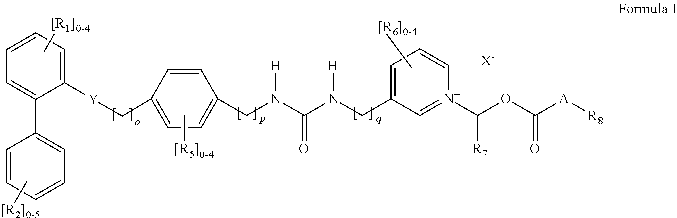

and pharmaceutically acceptable salts and solvates thereof; wherein R₁, R₂, R₅, R₆, R₇, R₈, A, X, Y, o, p, and q are as defined below.

The present invention further provides compounds of Formula Ia

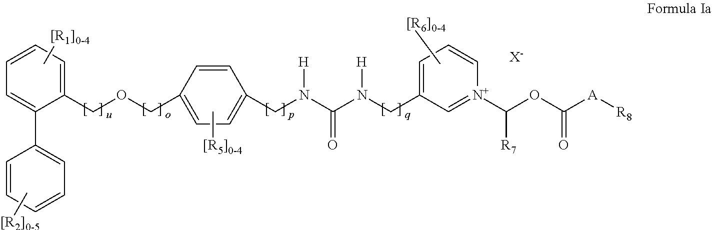

and pharmaceutically acceptable salts and solvates thereof; wherein R₁, R₂, R₅, R₆, R₇, R₈, A, X, u, o, p, and q are as defined below.

The present invention further provides compounds of Formula Ib Formula Ib

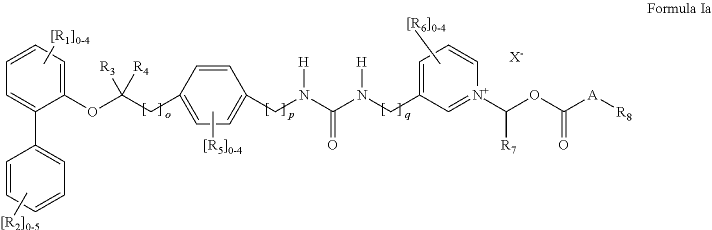

and pharmaceutically acceptable salts and solvates thereof; wherein R₁, R₂, R₃, R₄, R₅, R₆, R₇, R₈, A, X, o, p, and q are as defined below.

As noted above, the present invention provides chemical compounds that inhibit the activity of Nampt, and therefore can be used in the treatment of cancer, systemic or chronic inflammation, rheumatoid arthritis, diabetes, obesity, T-cell mediated autoimmune disease, ischemia, and other complications associated with these diseases and disorders. Thus, in a related aspect, the present invention also provides methods for treating cancer, systemic or chronic inflammation, rheumatoid arthritis, diabetes, obesity, T-cell mediated autoimmune disease, ischemia, and other complications associated with these diseases and disorders, by administering to a patient in need of such treatment a therapeutically effective amount of one or more of the compounds of the present invention.

Also provided is the use of the compounds of the present invention for the manufacture of a medicament useful for therapy, particularly for the treatment of cancer, systemic or chronic inflammation, rheumatoid arthritis, diabetes, obesity, T-cell mediated autoimmune disease, ischemia, and other complications associated with these diseases and disorders. In addition, the present invention also provides a pharmaceutical composition having one or more of the compounds of the present invention and one or more pharmaceutically acceptable excipients. Further, methods for the treatment of cancer, systemic or chronic inflammation, rheumatoid arthritis, diabetes, obesity, T-cell mediated autoimmune disease, ischemia, and other complications associated with these diseases and disorders, by administering to a patient in need of such treatment, a pharmaceutical composition of the present invention, is also encompassed.

In addition, the present invention further provides methods for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. These methods comprise administering an effective amount of one or more of the compounds of the present invention, preferably in the form of a pharmaceutical composition or medicament, to an individual having, or at risk of developing, cancer, systemic or chronic inflammation, rheumatoid arthritis, type 2 diabetes, obesity, T-cell mediated autoimmune disease, ischemia, and other complications associated with these diseases and disorders.

The compounds of the present invention can be used in combination therapies. Thus, combination therapy methods are also provided for treating or delaying the onset of the symptoms associated with cancer, systemic or chronic inflammation, rheumatoid arthritis, type 2 diabetes, obesity, T-cell mediated autoimmune disease, ischemia, and other complications associated with these diseases and disorders. Such methods comprise administering to a patient in need thereof one or more of the compounds of the present invention and, together or separately, at least one other anti-cancer, anti-inflammation, anti-rheumatoid arthritis, anti-type 2 diabetes, anti-obesity, anti-T-cell mediated autoimmune disease, or anti-ischemia therapy.

The present invention also provides a method of making a compound, comprising reacting a compound having a structure according to Parent Compound I with a desired Prodrug Moiety under suitable conditions to yield a compound having a structure according to Formula I.

The foregoing and other advantages and features of the embodiments of the present invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only, and are not intended to be limiting.

Other features and advantages of the invention will be apparent to one of skill in the art from the following detailed description, and from the claims below.

### BRIEF DESCRIPTION OF THE DRAWINGS

FIG. 1 depicts xenograft data for Parent Compound A formulated in Captisol®.

FIG. 2 depicts xenograft data for Example Compound 35.

FIG. 3 depicts xenograft data for Example Compound 42.

FIG. 4 depicts xenograft data for Example Compounds 44 and 45.

FIG. 5 depicts median concentration of Parent Compound A and Example Compound 35 in hepatic portal vein of rats following a single oral dose of Example Compound 35 (20 mg/kg).

FIG. 6 depicts median plasma concentration of Parent Compound A following a single IV dose of Parent Compound A (2.5 mg/kg) to rats.

FIG. 7 depicts a comparison of median concentration of Parent Compound A in hepatic portal vein samples following oral dose of Example Compound 35 at 20 mg/kg and femoral vein samples following an IV dose of Parent Compound A at 2.5 mg/kg.

### DETAILED DESCRIPTION OF THE INVENTION

### 1. Definitions

As used herein, the term “alkyl” as employed herein by itself or as part of another group refers to a saturated aliphatic hydrocarbon straight chain or branched chain group having, unless otherwise specified, 1 to 20 carbon atoms (whenever it appears herein, a numerical range such as “1 to 20” refers to each integer in the given range; e.g., “1 to 20 carbon atoms” means that the alkyl group can consist of 1, 2 or 3 carbon atoms, or more carbon atoms, up to a total of 20). An alkyl group can be in an unsubstituted form or substituted form with one or more substituents (generally one to three substitutents can be present except in the case of halogen substituents, e.g., perchloro). For example, a C₁₋₆ alkyl group refers to a straight or branched aliphatic group containing 1 to 6 carbon atoms (e.g., include methyl, ethyl, propyl, isopropyl, butyl, sec-butyl, tert-butyl, 3-pentyl, hexyl, etc.), which can be optionally substituted.

As used herein, “lower alkyl” refers to an alkyl group having from 1 to 6 carbon atoms.

The term “alkylene” as used herein means a saturated aliphatic hydrocarbon straight chain or branched chain group having from 1 to 20 carbon atoms having two connecting points (i.e., a “divalent” chain). For example, “ethylene” represents the group —CH₂—CH₂— and “methylene” represents the group —CH₂—. Alkylene chain groups can also be thought of as multiple methylene groups. For example, ethylene contains two methylene groups. Alkylene groups can also be in an unsubstituted form or substituted form with one or more substituents.

The term “alkenyl” as employed herein by itself or as part of another group means a straight or branched divalent chain radical of 2-10 carbon atoms (unless the chain length is otherwise specified), including at least one double bond between two of the carbon atoms in the chain. The alkenyl group can also be in an unsubstituted form or substituted form with one or more substituents (generally one to three substitutents except in the case of halogen substituents, e.g., perchloro or perfluoroalkyls). For example, a C₂₋₆ alkenyl group refers to a straight or branched chain radical containing 2 to 6 carbon atoms and having at least one double bond between two of the carbon atoms in the chain (e.g., ethenyl, 1-propenyl, 2-propenyl, 2-methyl-1-propenyl, 1-butenyl and 2-butenyl, which can be optionally substituted).

The term “alkenylene” as used herein means an alkenyl group having two connecting points. For example, “ethenylene” represents the group —CH═CH—. Alkenylene groups can also be in an unsubstituted form or substituted form with one or more substituents.

The term “alkynyl” as used herein by itself or as part of another group means a straight or branched chain radical of 2-10 carbon atoms (unless the chain length is otherwise specified), wherein at least one triple bond occurs between two of the carbon atoms in the chain. The alkynyl group can be in an unsubstituted form or substituted form with one or more substituents (generally one to three substitutents except in the case of halogen substituents, e.g., perchloro or perfluoroalkyls). For example, a C₂₋₆ alkynyl group refers to a straight or branched chain radical containing 2 to 6 carbon atoms, which can be optionally substituted, and having at least one triple bond between two of the carbon atoms in the chain (e.g., ethynyl, 1-propynyl, 1-methyl-2-propynyl, 2-propynyl, 1-butynyl and 2-butynyl).

The term “alkynylene” as used herein means an alkynyl having two connecting points. For example, “ethynylene” represents the group —C≡C—. Alkynylene groups can also be in an unsubstituted form or substituted form with one or more substituents.

The term “carbocycle” as used herein by itself or as part of another group means cycloalkyl and non-aromatic partially saturated carbocyclic groups such as cycloalkenyl and cycloalkynyl. A carbocycle can be in an unsubstituted form or substituted form with one or more substituents so long as the resulting compound is sufficiently stable and suitable for use in the embodiments of the present invention.

The term “cycloalkyl” as used herein by itself or as part of another group refers to a fully saturated 3- to 8-membered cyclic hydrocarbon ring (i.e., a cyclic form of an alkyl) alone (“monocyclic cycloalkyl”) or fused to another cycloalkyl, cycloalkynyl, cycloalkenyl, heterocycle, aryl or heteroaryl ring (i.e., sharing an adjacent pair of carbon atoms with other such rings) (“polycyclic cycloalkyl”). Thus, a cycloalkyl can exist as a monocyclic ring, bicyclic ring, or a spiral ring. When a cycloalkyl is referred to as a Cₓ cycloalkyl, this means a cycloalkyl in which the fully saturated cyclic hydrocarbon ring (which may or may not be fused to another ring) has x number of carbon atoms. When a cycloalkyl is recited as a substituent on a chemical entity, it is intended that the cycloalkyl moiety is attached to the entity through a single carbon atom within the fully saturated cyclic hydrocarbon ring of the cycloalkyl. In contrast, a substituent on a cycloalkyl can be attached to any carbon atom of the cycloalkyl. A cycloalkyl group can be unsubstituted or substituted with one or more substitutents so long as the resulting compound is sufficiently stable and suitable for use in the embodiments of the present invention. Examples of cycloalkyl groups include, for example, cyclopropyl, cyclobutyl, cyclopentyl, cyclohexyl and cycloheptyl.

The term “cycloalkenyl” as used herein by itself or as part of another group refers to a non-aromatic partially saturated 3- to 8-membered cyclic hydrocarbon ring having a double bond therein (i.e., a cyclic form of an alkenyl) alone (“monocyclic cycloalkenyl”) or fused to another cycloalkyl, cycloalkynyl, cycloalkenyl, heterocycle, aryl or heteroaryl ring (i.e., sharing an adjacent pair of carbon atoms with such other rings) (“polycyclic cycloalkenyl”). Thus, a cycloalkenyl can exist as a monocyclic ring, bicyclic ring, polycyclic or a spiral ring. When a cycloalkenyl is referred to as a Cₓ cycloalkenyl, this means a cycloalkenyl in which the non-aromatic partially saturated cyclic hydrocarbon ring (which may or may not be fused to another ring) has x number of carbon atoms. When a cycloalkenyl is recited as a substituent on a chemical entity, it is intended that the cycloalkenyl moiety is attached to the entity through a carbon atom within the non-aromatic partially saturated ring (having a double bond therein) of the cycloalkenyl. In contrast, a substituent on a cycloalkenyl can be attached to any carbon atom of the cycloalkenyl. A cycloalkenyl group can be in an unsubstituted form or substituted form with one or more substitutents. Examples of cycloalkenyl groups include cyclopentenyl, cycloheptenyl and cyclooctenyl.

The term “heterocycle” (or “heterocyclyl” or “heterocyclic” or “heterocyclo”) as used herein by itself or as part of another group means a saturated or partially saturated 3-7 membered non-aromatic cyclic ring formed with carbon atoms and from one to four heteroatoms independently selected from the group consisting of O, N, and S, wherein the nitrogen and sulfur heteroatoms can be optionally oxidized, and the nitrogen can be optionally quaternized (“monocyclic heterocycle”). The term “heterocycle” also encompasses a group having the non-aromatic heteroatom-containing cyclic ring above fused to another monocyclic cycloalkyl, cycloalkynyl, cycloalkenyl, heterocycle, aryl or heteroaryl ring (i.e., sharing an adjacent pair of atoms with such other rings) (“polycyclic heterocycle”). Thus, a heterocycle can exist as a monocyclic ring, bicyclic ring, polycyclic or a spiral ring. When a heterocycle is recited as a substituent on a chemical entity, it is intended that the heterocycle moiety is attached to the entity through an atom within the saturated or partially saturated ring of the heterocycle. In contrast, a substituent on a heterocycle can be attached to any suitable atom of the heterocycle. In a “saturated heterocycle” the non-aromatic heteroatom-containing cyclic ring described above is fully saturated, whereas a “partially saturated heterocyle” contains one or more double or triple bonds within the non-aromatic heteroatom-containing cyclic ring regardless of the other ring it is fused to. A heterocycle can be in an unsubstituted form or substituted form with one or more substituents so long as the resulting compound is sufficiently stable and suitable for use in the embodiments of the present invention.

Some examples of saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “aryl” by itself or as part of another group means an all-carbon aromatic ring with up to 7 carbon atoms in the ring (“monocylic aryl”). In addition to monocyclic aromatic rings, the term “aryl” also encompasses a group having the all-carbon aromatic ring above fused to another cycloalkyl, cycloalkynyl, cycloalkenyl, heterocycle, aryl or heteroaryl ring (i.e., sharing an adjacent pair of carbon atoms with such other rings) (“polycyclic aryl”). When an aryl is referred to as a Cₓ aryl, this means an aryl in which the all-carbon aromatic ring (which may or may not be fused to another ring) has x number of carbon atoms. When an aryl is recited as a substituent on a chemical entity, it is intended that the aryl moiety is attached to the entity through an atom within the all-carbon aromatic ring of the aryl. In contrast, a substituent on an aryl can be attached to any suitable atom of the aryl. Examples, without limitation, of aryl groups are phenyl, naphthalenyl and anthracenyl. An aryl can be in an unsubstituted form or substituted form with one or more substituents so long as the resulting compound is sufficiently stable and suitable for use in the embodiments of the present invention.

The term “heteroaryl” as employed herein refers to a stable aromatic ring having up to 7 ring atoms with 1, 2, 3 or 4 hetero ring actoms in the ring which are oxygen, nitrogen or sulfur or a combination thereof (“monocylic heteroaryl”). In addition to monocyclic hetero-aromatic rings, the term “heteroaryl” also encompasses a group having the monocyclic hetero-aromatic ring above fused to another cycloalkyl, cycloalkynyl, cycloalkenyl, heterocycle, aryl or heteroaryl ring (i.e., sharing an adjacent pair of atoms with such other rings) (“polycyclic heteroaryl”). When a heteroaryl is recited as a substituent on a chemical entity, it is intended that the heteroaryl moiety is attached to the entity through an atom within the heteroaromatic ring of the heteroaryl. In contrast, a substituent on a heteroaryl can be attached to any suitable atom of the heteroaryl. A heteroaryl can be in an unsubstituted form or substituted form with one or more substituents so long as the resulting compound is sufficiently stable and suitable for use in the embodiments of the present invention.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, (3-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom can be in the form of an N-oxide, e.g., a pyridyl N-oxide, pyrazinyl N-oxide and pyrimidinyl N-oxide.

As used herein, the term “halo” refers to chloro, fluoro, bromo, or iodo substitutents.

As used herein, the term “hydro” refers to a bound hydrogen atom (—H group).

As used herein, the term “hydroxyl” refers to an —OH group.

As used herein, the term “alkoxy” refers to an —O—(C₁₋₁₂ alkyl). Lower alkoxy refers to —O— (lower alkyl) groups.

As used herein, the term “alkynyloxy” refers to an —O—(C₂₋₁₂ alkynyl).

As used herein, the term “cycloalkyloxy” refers to an —O-cycloalkyl group.

As used herein, the term “heterocycloxy” refers to an —O-heterocycle group.

As used herein, the term “aryloxy” refers to an —O-aryl group. Examples of aryloxy groups include, but are not limited to, phenoxy and 4-methylphenoxy.

The term “heteroaryloxy” refers to an —O-heteroaryl group.

The terms “arylalkoxy” and “heteroarylalkoxy” are used herein to mean alkoxy group substituted with an aryl group and a heteroaryl group, respectively. Examples of arylalkoxy groups include, but are not limited to, benzyloxy and phenethyloxy.

As used herein, the term “mercapto” or “thiol” group refers to an —SH group.

The term “alkylthio” group refers to an —S-alkyl group.

The term “arylthio” group refers to an —S-aryl group.

The term “arylalkyl” is used herein to mean above-defined alkyl group substituted by an aryl group defined above. Examples of arylalkyl groups include benzyl, phenethyl and naphthylmethyl, etc. An arylalkyl group can be unsubstituted or substituted with one or more substituents so long as the resulting compound is sufficiently stable and suitable for use in the embodiments of the present invention.

The term “heteroarylalkyl” is used herein to mean an alkyl group, as defined above, substituted by any heteroaryl group. A heteroarylalkyl can be unsubstituted or substituted with one or more substituents, so long as the resulting compound is sufficiently stable and suitable for use in the embodiments of the present invention.

The term “heteroarylalkenyl” is used herein to mean any of the above-defined alkenyl groups substituted by any of the above-defined heteroaryl groups.

The term “arylalkynyl” is used herein to mean any of the above-defined alkynyl groups substituted by any of the above-defined aryl groups.

The term “heteroarylalkenyl” is used herein to mean any of the above-defined alkenyl groups substituted by any of the above-defined heteroaryl groups.

The term “arylalkoxy” is used herein to mean alkoxy group substituted by an aryl group as defined above.

“Heteroarylalkoxy” is used herein to mean any of the above-defined alkoxy groups substituted by any of the above-defined heteroaryl groups.

“Haloalkyl” means an alkyl group that is substituted with one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has an oxygen doubly-bonded to it; i.e. one of the ring carbon atoms is a —C(═O) group.

As used herein, the term “thiocarbonyl” group refers to a —C(═S)R″ group, with R″ as defined herein.

“Alkanoyl” refers to an —C(═O)-alkyl group.

The term “heterocyclonoyl” group refers to a heterocyclo group linked to the alkyl chain of an alkanoyl group.

The term “acetyl” group refers to a —C(═O)CH₃ group.

“Alkylthiocarbonyl” refers to an —C(═S)-alkyl group.

The term “cycloketone” refers to a carbocycle or heterocycle group in which one of the carbon atoms which form the ring has an oxygen doubly-bonded to it; i.e., one of the ring carbon atoms is a —C(═O) group.

The term “O-carboxy” group refers to a —OC(═O)R″ group, where R″ is as defined herein.

The term “C-carboxy” group refers to a —C(═O)OR″ groups where R″ is as defined herein.

As used herein, the term “carboxylic acid” refers to a C-carboxy group in which R″ is hydro. In other words, the term “carboxylic acid” refers to —COOH.

As used herein, the term “ester” is a C-carboxy group, as defined herein, wherein R″ is as defined above, except that it is not hydro (e.g., it is methyl, ethyl, or lower alkyl).

As used herein, the term “C-carboxy salt” refers to a —C(═O)O⁻ M⁺ group wherein M⁺ is selected from the group consisting of lithium, sodium, magnesium, calcium, potassium, barium, iron, zinc and quaternary ammonium.

The term “carboxyalkyl” refers to —C₁₋₆ alkylene-C(═O)OR″ (that is, a C₁₋₆ alkyl group connected to the main structure wherein the alkyl group is substituted wth —C(═O)OR″ with R″ being defined herein). Examples of carboxyalkyl include, but are not limited to, —CH₂COOH, —(CH₂)₂COOH, —(CH₂)₃COOH, —(CH₂)₄COOH, and —(CH₂)₅COOH.

“Carboxyalkenyl” refers to -alkenylene-C(═O)OR″ with R″ being defined herein.

The term “carboxyalkyl salt” refers to a —(CH₂)ᵣC(═O)O-M⁺ wherein M⁺ is selected from the group consisting of lithium, sodium, potassium, calcium, magnesium, barium, iron, zinc and quaternary ammonium, and wherein r is 1-6.

The term “carboxyalkoxy” refers to —O—(CH₂)ᵣC(═O)OR″ wherein r is 1-6, and R″ is as defined herein.

“Cₓ carboxyalkanoyl” means a carbonyl group (—(O═)C—) attached to an alkyl or cycloalkylalkyl group that is substituted with a carboxylic acid or carboxyalkyl group, wherein the total number of carbon atom is x (an integer of 2 or greater).

“Cₓ carboxyalkenoyl” means a carbonyl group (—(O═)C—) attached to an alkenyl or alkyl or cycloalkylalkyl group that is substituted with a carboxylic acid or carboxyalkyl or carboxyalkenyl group, wherein at least one double bond (—CH═CH—) is present and wherein the total number of carbon atom is x (an integer of 2 or greater).

“Carboxyalkoxyalkanoyl” means refers to R″OC(═O)—C₁₋₆ alkylene-O—C₁₋₆ alkylene-C(═O)—, R″ is as defined herein.

“Amino” refers to an —NR^{x}R_{y} group, with R^{x} and R^{y} as defined herein.

“Alkylamino” means an amino group with a substituent being a C₁₋₆ alkyl.

“Aminoalkyl” means an alkyl group connected to the main structure of a molecule where the alkyl group has a substituent being amino.

“Quaternary ammonium” refers to a -⁺N(R^{x})(R^{y})(R^{z}) group wherein R^{x}, R^{y}, and R^{z} are as defined herein.

The term “nitro” refers to a —NO₂ group.

The term “O-carbamyl” refers to a —OC(═O)N(R^{x})(R^{y}) group with R^{x} and R^{y} as defined herein.

The term “N-carbamyl” refers to a R^{y} OC(═O)N(R^{x})— group, with R^{x} and R^{y} as defined herein.

The term “O-thiocarbamyl” refers to a —OC(═S)N(R^{x})(R^{y}) group with R^{x} and R^{y} as defined herein.

The term “N-thiocarbamyl” refers to a R^{x}OC(═S)NR⁻ group, with R^{x} and R^{y} as defined herein.

“C-amido” refers to a —C(═O)N(R^{x})(R^{y}) group with R^{x} and R^{y} as defined herein.

“N-amido” refers to a R^{x}C(═O)N(R^{y})— group with R^{x} and R^{y} as defined herein.

“Aminothiocarbonyl” refers to a —C(═S)N(R^{x})(R^{y}) group with R^{x} and R^{y} as defined herein.

“Hydroxyaminocarbonyl” means a —C(═O)N(R^{x})(OH) group with R^{x} as defined herein.

“Alkoxyaminocarbonyl” means a —C(═O)N(R^{x})(alkoxy) group with R^{x} as defined herein.

The terms “cyano” and “cyanyl” refer to a —C≡N group.

The term “nitrile” group, as used herein, refers to a —C≡N substituent.

The term “cyanato” refers to a —CNO group.

The term “isocyanato” refers to a —NCO group.

The term “thiocyanato” refers to a —CNS group.

The term “isothiocyanato” refers to a —NCS group.

The term “oxo” refers to a —C(═O)— group.

The term “sulfinyl” refers to a —S(═O)R″ group, where R″ is as defined herein.

The term “sulfonyl” refers to a —S(═O)₂R″ group, where R″ is as defined herein.

The term “sulfonamide” refers to a —(R^{x})N—S(═O)₂R″ group, with R″ and R^{x} as defined herein.

“Aminosulfonyl” means (R^{x})(R^{y})N—S(═O)₂— with R^{x} and R^{y} as defined herein.

“Aminosulfonyloxy” means a (R^{x})(R^{y})N—S(═O)₂—O⁻ group with R^{x} and R^{y} as defined herein.

“Sulfonamidecarbonyl” means R″—S(═O)₂—N(R^{x})—C(═O)⁻ with R″ and R^{x} as defined herein.

“Alkanoylaminosulfonyl” refers to an alkyl-C(═O)—N(R^{x})—S(═O)₂— group with R^{x} as defined herein.

The term “trihalomethylsulfonyl” refers to a X₃CS(═O)₂— group with X being halo.

The term “trihalomethylsulfonamide” refers to a X₃CS(═O)₂N(R^{x})— group with X being halo and R^{x} as defined herein.

R″ is selected from the group consisting of hydro, alkyl, cycloalkyl, aryl, heteroaryl and heterocycle, each being optionally substituted.

R^{x}, R^{y}, and R^{z} are independently selected from the group consisting of hydro and optionally substituted alkyl.

The term “methylenedioxy” refers to a —OCH₂O— group wherein the oxygen atoms are bonded to adjacent ring carbon atoms.

The term “ethylenedioxy” refers to a —OCH₂CH₂O— group wherein the oxygen atoms are bonded to adjacent ring carbon atoms.

As used herein, the phrase “optionally substituted” means substituted or unsubstituted.

Unless specifically stated otherwise or indicated by a bond symbol (dash, double dash, or triple dash), the connecting point to a recited group will be on the right-most stated group. Thus, for example, a hydroxyalkyl group is connected to the main structure through the alkyl and the hydroxyl is a substituent on the alkyl.

### 2. Therapeutic Compounds

The present invention provides chemical compounds that selectively inhibit the activity of Nampt. These compounds can be used in the treatment of cancer, systemic or chronic inflammation, rheumatoid arthritis, diabetes, obesity, T-cell mediated autoimmune disease, ischemia, and other complications associated with these diseases and disorders.

In some embodiments the present invention provides compounds of Formula I

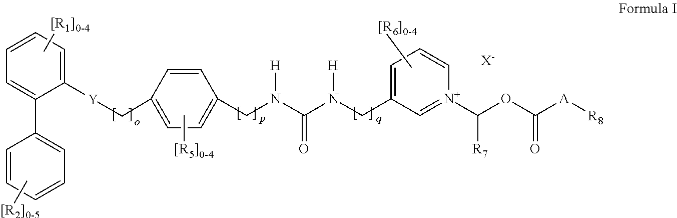

and pharmaceutically acceptable salts and solvates thereof;
wherein:

X is a pharmaceutically-acceptable counterion, such as, by way of non-limiting example, halides, such as fluoride, chloride, bromide, and iodide, mesylates, tosylates, p-toluenesulfonate, nitrates, carboxylates, such as acetate, and phosphates;

Y is —CH₂CH₂O—, —CH₂O—, —OCH₂—, —SCH₂—, —N(R)CH₂—, —N(R)C(═O)—, —C(═O)N(R)—, —S(═O)₂CH₂—, —S(═O)CH₂—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)—, —O—C₁₋₄ alkylene-C(═O)N(R)—, —N(R)C(═O)—C₁₋₄ alkylene-O—, —C(═O)N(R)—C₁₋₄ alkylene-O—, —C₁₋₄ alkylene-S(═O)₂—, —C₁₋₄ alkylene-S(═O)—, —S(═O)₂—C₁₋₄ alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —S—C₁₋₄ alkylene-, —C₁₋₄ alkylene-S—, —C₁₋₄ alkylene-S—C₁₋₄ alkylene-, —N(R)—C₁₋₄ alkylene-, —C₁₋₄ alkylene-N(R)—, —C₁₋₄ alkylene-N(R)—C₁₋₄ alkylene-, —C₁₋₄ alkylene-C(═O)—O—C₁₋₄ alkylene-, —C₁₋₄ alkylene-O—C(═O)—C₁₋₄ alkylene-, —C₁₋₄ alkylene-C(═O)—N(R)—C₁₋₄ alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;

R₁ and R₂, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heterocyclo, cycloalkyl, or amino;

R₅, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

R₆, if present one or more times, is only attached to a ring carbon and is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

A is optionally present and if present is selected from O, S, N(R₁₁), N(R₁₁)—C₁₋₄ alkylene, and C₁₋₄ alkylene;

R₁₁ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₇ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₈ is selected from optionally-substituted C₁₋₄ alkyl, optionally-substituted C₁₋₄ alkoxy, optionally-substituted C-carboxy, optionally-substituted aryl, optionally-substituted heteroaryl, optionally-substituted cycloalkyl, and optionally-substituted heterocyclyl; o, p, and q are each independently 0, 1, or 2; and any methylene group of the o, p, and q regions, Y, and A, are each optionally independently substituted with C₁₋₄ alkyl, halo, C₁₋₄ haloalkyl, or C₃ or C₄ cycloalkyl.

In some embodiments of the compounds of Formula I R for the purposes of Y is hydrogen.

In some embodiments of the compounds of Formula I, Y₂ is —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, —O—C₁₋₄ alkylene-N(R)C(═O)—, —C₁₋₄ alkylene-S(═O)₂—, —C₁₋₄ alkylene-S(═O)—, —S(═O)₂—C₁₋₄ alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —C₁₋₄ alkylene-S—, —C₁₋₄ alkylene-S—C₁₋₄ alkylene-, —C₁₋₄ alkylene-N(R)—, —C₁₋₄ alkylene-N(R)—C₁₋₄ alkylene-, —C₁₋₄ alkylene-C(═O)—O—C₁₋₄ alkylene-, —C₁₋₄ alkylene-O—C(═O)—C₁₋₄ alkylene-, —C₁₋₄ alkylene-C(═O)—N(R)—C₁₋₄ alkylene-, or —C₁₋₄ alkylene-N(R)—C(═O)—C₁₋₄ alkylene-, wherein R is H, halo, C₁₋₅ alkyl, C₁₋₅ alkenyl, or C₁₋₅ alkynyl.

In some embodiments of the compounds of Formula I, Y₂ is —OCH₂—, —SCH₂—, —N(R)CH₂—, —CH₂O—, —CH₂CH₂O—, —CH₂S—, —CH₂N(R)—, —SO₂N(R)—, —N(R)SO₂—, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —S—C₁₋₄ alkylene-, —C₁₋₄ alkylene-S—, —C₁₋₄ alkylene-S—C₁₋₄ alkylene-, —N(R)—C₁₋₄ alkylene-, —C₁₋₄ alkylene-N(R)—, or —C₁₋₄ alkylene-N(R)—C₁₋₄ alkylene-, wherein R is H, halo, C₁₋₅ alkyl, C₁₋₅ alkenyl, or C₁₋₅ alkynyl.

In some embodiments of the compounds of Formula I, Y is —S(═O)₂CH₂—.

In some embodiments of the compounds of Formula I, Y is —S(═O)CH₂—.

In some embodiments of the compounds of Formula I, Y is —CH₂S—.

In some embodiments of the compounds of Formula I, Y is —CH₂N(R)—, wherein R is H, halo, C₁₋₅ alkyl, C₁₋₅ alkenyl, or C₁₋₅ alkynyl.

In some embodiments of the compounds of Formula I, Y is —CH₂S(═O)₂—.

In some embodiments of the compounds of Formula I, Y is —CH₂S(═O)—.

In some embodiments of the compounds of Formula I, Y is —C(═O)O—.

In some embodiments of the compounds of Formula I, Y is —OC(═O)—.

In some embodiments of the compounds of Formula I, Y is —N(R)SO₂—, wherein R is H, halo, C₁₋₅ alkyl, C₁₋₅ alkenyl, or C₁₋₅ alkynyl.

In some embodiments of the compounds of Formula I, Y is ethylene.

In some embodiments of the compounds of Formula I, Y is propylene.

In some embodiments of the compounds of Formula I, Y is n-butylene.

In some embodiments of the compounds of Formula I, Y is —O—C₁₋₄ alkylene-N(R)C(═O)—, wherein R is H, halo, C₁₋₅ alkyl, C₁₋₅ alkenyl, or C₁₋₅ alkynyl.

In some embodiments of the compounds of Formula I, Y is —O—C₁₋₄ alkylene-C(═O)N(R)—, wherein R is H, halo, C₁₋₅ alkyl, C₁₋₅ alkenyl, or C₁₋₅ alkynyl.

In some embodiments of the compounds of Formula I, Y is —N(R)C(═O)—C₁₋₄ alkylene-O—, wherein R is H, halo, C₁₋₅ alkyl, C₁₋₅ alkenyl, or C₁₋₅ alkynyl.

In some embodiments of the compounds of Formula I, wherein R is H, halo, C₁₋₅ alkyl, C₁₋₅ alkenyl, or C₁₋₅ alkynyl.

In some embodiments of the compounds of Formula I, Y is —C₁₋₄ alkylene-S(═O)₂—.

In some embodiments of the compounds of Formula I, Y is —C₁₋₄ alkylene-S(═O)—.

In some embodiments of the compounds of Formula I, Y is —S(═O)₂—C₁₋₄ alkylene-.

In some embodiments of the compounds of Formula I, Y is —S(═O)—C₁₋₄ alkylene-.

In some embodiments of the compounds of Formula I, Y is —C₁₋₄ alkylene-SO₂N(R)—, wherein R is H, halo, C₁₋₅ alkyl, C₁₋₅ alkenyl, or C₁₋₅ alkynyl.

In some embodiments of the compounds of Formula I, Y is —C₁₋₄ alkylene-N(R)SO₂—, wherein R is H, halo, C₁₋₅ alkyl, C₁₋₅ alkenyl, or C₁₋₅ alkynyl.

In some embodiments of the compounds of Formula I, Y is —SO₂N(R)—C₁₋₄ alkylene-, wherein R is H, halo, C₁₋₅ alkyl, C₁₋₅ alkenyl, or C₁₋₅ alkynyl.

In some embodiments of the compounds of Formula I, Y is —N(R)SO₂—C₁₋₄ alkylene-, wherein R is H, halo, C₁₋₅ alkyl, C₁₋₅ alkenyl, or C₁₋₅ alkynyl.

In some embodiments of the compounds of Formula I, Y is —C₁₋₄ alkylene-O—C₁₋₄ alkylene-.

In some embodiments of the compounds of Formula I, Y is —O—C₁₋₄ alkylene-.

In some embodiments of the compounds of Formula I, Y is —C₁₋₄ alkylene-O—.

In some embodiments of the compounds of Formula I, Y is —S—C₁₋₄ alkylene-.

In some embodiments of the compounds of Formula I, Y is —C₁₋₄ alkylene-S—.

In some embodiments of the compounds of Formula I, Y is —C₁₋₄ alkylene-S—C₁₋₄ alkylene-.

In some embodiments of the compounds of Formula I, Y is —N(R)—C₁₋₄ alkylene-, wherein R is H, halo, C₁₋₅ alkyl, C₁₋₅ alkenyl, or C₁₋₅ alkynyl.

In some embodiments of the compounds of, Y is —C₁₋₄ alkylene-N(R)—, wherein R is H, halo, C₁₋₅ alkyl, C₁₋₅ alkenyl, or C₁₋₅ alkynyl.

In some embodiments of the compounds of Formula I, Y is —C₁₋₄ alkylene-N(R)—C₁₋₄ alkylene-, wherein R is H, halo, C₁₋₅ alkyl, C₁₋₅ alkenyl, or C₁₋₅ alkynyl.

In some embodiments of the compounds of Formula I, Y is —C₁₋₄ alkylene-C(═O)—O—C₁₋₄ alkylene-.

In some embodiments of the compounds of Formula I, Y is —C₁₋₄ alkylene-O—C(═O)—C₁₋₄ alkylene-.

In some embodiments of the compounds of Formula I, Y is —C₁₋₄ alkylene-C(═O)—N(R)—C₁₋₄ alkylene-, wherein R is H, halo, C₁₋₅ alkyl, C₁₋₅ alkenyl, or C₁₋₅ alkynyl.

In some embodiments of the compounds of Formula I, Y is —C₁₋₄ alkylene-N(R)—C(═O)—C₁₋₄ alkylene-, wherein R is H, halo, C₁₋₅ alkyl, C₁₋₅ alkenyl, or C₁₋₅ alkynyl.

In some embodiments of the compounds of Formula I, Y is —SCH₂—.

In some embodiments of the compounds of Formula I, Y is —N(R)CH₂—, wherein R is H, halo, C₁₋₅ alkyl, C₁₋₅ alkenyl, or C₁₋₅ alkynyl.

In some embodiments of the compounds of Formula I, Y is —N(R)C(═O)—, wherein R is H, halo, C₁₋₅ alkyl, C₁₋₅ alkenyl, or C₁₋₅ alkynyl.

In some embodiments of the compounds of Formula I, Y is —C(═O)N(R)—, wherein R is H, halo, C₁₋₅ alkyl, C₁₋₅ alkenyl, or C₁₋₅ alkynyl.

In some embodiments the present invention provides compounds of Formula Ia

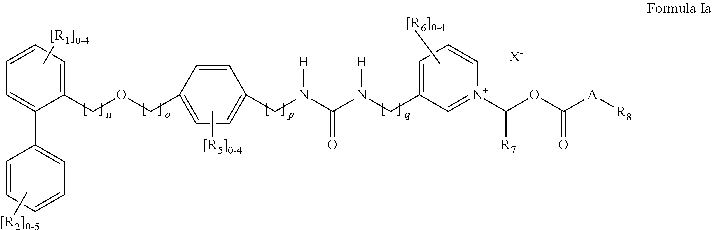

and pharmaceutically acceptable salts and solvates thereof;
wherein:

X is a pharmaceutically-acceptable counterion, such as, by way of non-limiting example, halides, such as fluoride, chloride, bromide, and iodide, mesylates, tosylates, p-toluenesulfonate, nitrates, carboxylates, such as acetate, and phosphates;

R₁ and R₂, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heterocyclo, cycloalkyl, or amino;

R₅, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

R₆, if present one or more times, is only attached to a ring carbon and is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

A is optionally present and if present is selected from O, S, N(R₁₁), N(R₁₁)—C₁₋₄ alkylene, and C₁₋₄ alkylene;

R₁₁ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₇ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₈ is selected from optionally-substituted C₁₋₄ alkyl, optionally-substituted C₁₋₄ alkoxy, optionally-substituted C-carboxy, optionally-substituted aryl, optionally-substituted heteroaryl, optionally-substituted cycloalkyl, and optionally-substituted heterocyclyl;

o, p, and q are each independently 0, 1, or 2;

u is 1 or 2; and

any methylene group of the o, p, q, and u regions and A are each optionally independently substituted with C₁₋₄ alkyl, halo, C₁₋₄ haloalkyl, or C₃ or C₄ cycloalkyl.

In some embodiments of the compounds of Formula Ia, q is 1, p is 0, o is 0, u is 2, and R₁, R₂, R₅ and R₆ are not present.

In some embodiments the present invention provides compounds of Formula Ib

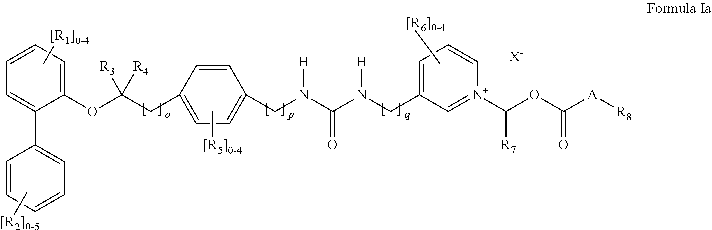

and pharmaceutically acceptable salts and solvates thereof;
wherein:

X is a pharmaceutically-acceptable counterion, such as, by way of non-limiting example, halides, such as fluoride, chloride, bromide, and iodide, mesylates, tosylates, p-toluenesulfonate, nitrates, carboxylates, such as acetate, and phosphates;

R₁ and R₂, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heterocyclo, cycloalkyl, or amino;

R₃ and R₄ are each independently H, halo, or C₁₋₄ alkyl, or R₃ and R₄ taken together form a cyclopropyl or cyclobutyl ring;

R₅, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

R₆, if present one or more times, is only attached to a ring carbon and is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;

A is optionally present and if present is selected from O, S, N(R₁₁), N(R₁₁)—C₁₋₄ alkylene, and C₁₋₄ alkylene;

R₁₁ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₇ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;

R₈ is selected from optionally-substituted C₁₋₄ alkyl, optionally-substituted C₁₋₄ alkoxy, optionally-substituted C-carboxy, optionally-substituted aryl, optionally-substituted heteroaryl, optionally-substituted cycloalkyl, and optionally-substituted heterocyclyl;

o, p, and q are each independently 0, 1, or 2; and

any methylene group of the o, p, and q regions and A, are each optionally independently substituted with C₁₋₄ alkyl, halo, C₁₋₄ haloalkyl, or C₃ or C₄ cycloalkyl.

In some embodiments of the compounds of Formula Ib, R₃ and R₄ are both hydrogen or both fluoro. In some of such embodiments, R₃ and R₄ are both hydrogen.

In some embodiments of the compounds of Formula Ib, q is 1, p is 0, o is 0, R₃ and R₄ are both hydro, and R₁, R₂, R₅ and R₆ are not present.

In some embodiments of the compounds of each of Formulae I, Ia, and Ib, R₁ is not present, or is present one, two, three, or four times. In some embodiments of the compounds of each of Formulae I, Ia, and Ib, R₁ is an electron-withdrawing group, such as by way of non-limiting example, halo, trihalomethyl, nitro, cyano, C-carboxy, O-carboxy, C-amido, and N-amido. In some embodiments of the compounds of each of Formulae I, Ia, and Ib, R₁ is selected from C₁₋₅ alkyl, C₁₋₅ alkoxy, C-amido, N-amido, amino, aminoalkyl, and alkylthio, each further substituted with heterocyclo, cycloalkyl, or amino.

In some embodiments of the compounds of each of Formulae I, Ia, and Ib, R₂ is not present or is present, one, two, three, four, or five times. In some embodiments of the compounds of each of Formulae I, Ia, and Ib, R₂ is selected from C₁₋₅ alkyl, C₁₋₅ alkoxy, C-amido, N-amido, amino, aminoalkyl, or alkylthio, each further substituted with heterocyclo, cycloalkyl, or amino.

In some embodiments of the compounds of each of Formulae I, Ia, and Ib, R₁ is selected from the following:

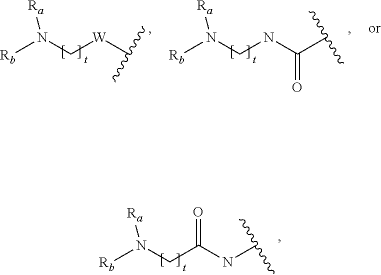

wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine.

In some embodiments of the compounds of each of Formulae I, Ia, and Ib, R₂ is selected from the following:

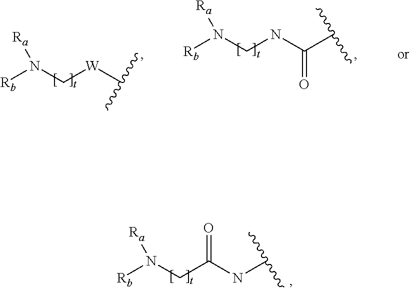

wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine.

In some embodiments of the compounds of each of Formulae I, Ia, and Ib, R₁ and/or R₂ is present and is located on the biphenyl ring as shown below:

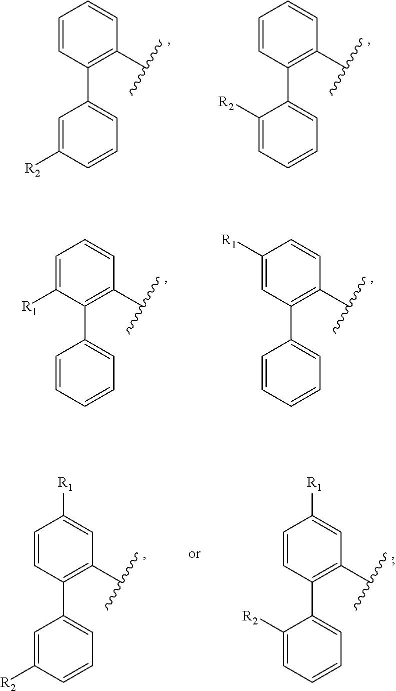

wherein R₁ and R₂ are each selected from the following:

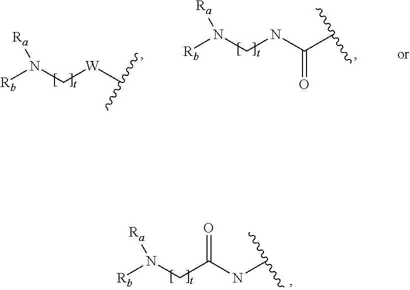

wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine; with the proviso that when R₁ and R₂ are both present on the biphenyl ring, then R₁ is C₁₋₄ haloalkyl (such as, for example, trifluoromethyl) or halo (such as, for example, chloro).

In some embodiments of the compounds of each of Formulae I, Ia, and Ib, R₅ is not present, or is present one, two, three, or four times. In some of such embodiments R₅, is not present or is fluoro, methyl, or trifluormethyl. In some of such embodiments R₅ is not present.

In some embodiments of the compounds of each of Formulae I, Ia, and Ib, o is 0. In some embodiments of the compounds of each of Formulae I, Ia, and Ib, o is 1. In some embodiments of the compounds of each of Formulae I, Ia, and Ib, o is 2. In some embodiments of the compounds of each of Formulae I, Ia, and Ib, any methylene groups of the o region are optionally substituted with fluoro or methyl. In some embodiments of the compounds of each of Formulae I, Ia, and Ib, any methylene groups of the o region are all fully saturated.

In some embodiments of the compounds of each of Formulae I, Ia, and Ib, p is 0. In some embodiments of the compounds of each of Formulae I, Ia, and Ib, p is 1. In some embodiments of the compounds of each of Formulae I, Ia, and Ib, p is 2. In some embodiments of the compounds of each of Formulae I, Ia, and Ib, any methylene groups of the p region are optionally substituted with fluoro or methyl. In some embodiments of the compounds of each of Formulae I, Ia, and Ib, any methylene groups of the p region are all fully saturated.

In some embodiments of the compounds of each of Formulae I, Ia, and Ib, q is 0. In some embodiments of the compounds of each of Formulae I, Ia, and Ib, q is 1. In some embodiments of the compounds of each of Formulae I, Ia, and Ib, q is 2. In some embodiments of the compounds of each of Formulae I, Ia, and Ib, any methylene groups of the q region are optionally substituted with fluoro or methyl. In some embodiments of the compounds of each of Formulae I, Ia, and Ib, any methylene groups of the q region are all fully saturated.

In some embodiments of the compounds of Formula Ia, u is 1. In some embodiments of the compounds of Formula Ia, u is 2. In some embodiments of the compounds of Formula Ia, the methylene group(s) of the u region are optionally substituted with fluoro or methyl. In some embodiments of the compounds of Formula Ia, the methylene group(s) of the u region are all fully saturated.

In some embodiments of the compounds of each of Formulae I, Ia, and Ib, any methylene groups are all fully saturated.

In some embodiments of the compounds of each of Formulae I, Ia, and Ib, q is 1, and p is 0.

In some embodiments of the compounds of each of Formulae I, Ia, and Ib, q is 1, p is 0, and o is 0.

In some embodiments of the compounds of each of Formulae Formulae I, Ia, and Ib, R₆ is no present, q is 1, p is 0, and o is 0.

In some embodiments of the compounds of each of Formulae I, Ia, and Ib, q is 1, p is 0, o is 0, and R₅ and R₆ are both not present.

In some embodiments of the compounds of each of Formulae I, Ia, and Ib, q is 1, p is 0, o is 0, and R₁, R₅, and R₆ are not present.

In some embodiments of the compounds of each of Formulae I, Ia, and Ib, q is 1, p is 0, o is 0, and R₁, R₂, R₅ and R₆ are not present.

In some embodiments of the compounds of each of Formulae I, Ia, and Ib, A is optionally present and if present is selected from O, S, N(R₁₁), N(R₁₁)—CH₂, N(R₁₁)—CH₂CH₂, methylene, and ethylene. In some of such embodiments R₁₁ is selected from hydro and C₁₋₄ alkyl.

In some embodiments of the compounds of each of Formulae I, Ia, and Ib, R₇ is selected from hydro and C₁₋₄ alkyl (including, by way of non-limiting example, methyl, ethyl, isopropyl, and t-butyl).

In some embodiments of the compounds of each of Formulae I, Ia, and Ib, R₈ is selected from C₁₋₄ alkyl, C₁₋₄ alkoxy, methoxyethoxyethoxyethoxyethoxy, C-carboxy, aryl, heteroaryl, cycloalkyl, and heterocyclyl, wherein said aryl, heteroaryl, cycloalkyl, and heterocyclyl are each optionally-substituted with C-carboxy, O-carboxy, C₁₋₄ O-carboxyalkylene, hydroxyl, or hydroxylalkylene.

In some embodiments of the compounds of each of Formulae I, Ia, and Ib, R₈ is selected from C₁₋₄ alkyl, C₁₋₄ alkoxy, methoxyethoxyethoxyethoxyethoxy, C-carboxy, phenyl, pyridinyl, cyclohexyl, piperidinyl, pyrrolidinyl, and morpholino, wherein said phenyl, pyridinyl, cyclohexyl, piperidinyl, pyrrolidinyl, and morpholino are each optionally-substituted with C-carboxy, O-carboxy, O-carboxyalkylene, hydroxyl, or hydroxylalkylene.

The compounds of the present invention include the compounds of Formulae I, Ia, and Ib, as illustrated herein, and the compounds of Tables 1A and 1B, as well as for any of the foregoing their stereochemically isomeric forms thereof. The compounds of the present invention also include pharmaceutically acceptable salts, prodrugs, N-oxide forms, quaternary amines, and solvates of the compounds of Formulae I, Ia, and Ib, as illustrated herein, and the compounds of Tables 1A and 1B.

For therapeutic use, salts of the compounds of Formulae I, Ia, and Ib, as illustrated herein, and the compounds of Tables 1A and 1B, are those particular salts wherein the counterion is pharmaceutically acceptable. However, salts of acids and bases which are non-pharmaceutically acceptable can also find use, for example, in the preparation or purification of a pharmaceutically acceptable compound. All salts, whether pharmaceutically acceptable or not, are within the ambit of the present invention.

The pharmaceutically acceptable addition salts as mentioned herein are meant to comprise the therapeutically active non-toxic acid addition salt forms which the compounds of Formulae I, Ia, and Ib, as illustrated herein, and the compounds of Tables 1A and 1B, are able to form. The salts can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted by treatment with alkali into the free base form.

The compounds of Formulae I, Ia, and Ib, as illustrated herein, and the compounds of Tables 1A and 1B, containing acidic protons can be converted into their therapeutically active non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanedi-ol, hydrabamine salts, and salts with amino acids such as, for example, arginine, lysine and the like. Conversely the salt form can be converted by treatment with acid into the free acid form.

The term addition salt also comprises the hydrates and solvent addition forms which the compounds of Formulae I, Ia, and Ib, as illustrated herein, and the compounds of Tables 1A and 1B, are able to form. Examples of such forms are e.g. hydrates, alcoholates and the like.

The term “quaternary amine” as used herein defines the quaternary ammonium salts which the compounds of Formulae I, Ia, and Ib, as illustrated herein, and the compounds of Tables 1A and 1B, are able to form by reaction between a basic nitrogen of one of the compounds of Formulae I, Ia, and Ib, as illustrated herein, and the compounds of Tables 1A and 1B, and an appropriate quatemizing agent, such as, for example, an optionally substituted alkylhalide, arylhalide or arylalkylhalide, e.g. methyliodide or benzyliodide. Other reactants with good leaving groups can also be used, such as alkyl trifluoromethanesulfonates, alkyl methanesulfonates, and alkyl p-toluenesulfonates. A quatemary amine has a positively charged nitrogen. Pharmaceutically acceptable counterions include chloro, bromo, iodo, trifluoroacetate and acetate. The counterion of choice can be introduced using ion exchange resins.

Pharmaceutically acceptable salts of the compounds of Formulae I, Ia, and Ib, as illustrated herein, and the compounds of Tables 1A and 1B, include all salts are exemplified by alkaline salts with an inorganic acid and/or a salt with an organic acid that are known in the art. In addition, pharmaceutically acceptable salts include acid salts of inorganic bases, as well as acid salts of organic bases. Their hydrates, solvates, and the like are also encompassed in the present invention. In addition, N-oxide compounds are also encompassed in the present invention.

It will be appreciated that some of the compounds of Formulae I, Ia, and Ib, as illustrated herein, and the compounds of Tables 1A and 1B, and their N-oxides, addition salts, quaternary amines and stereochemically isomeric forms can contain one or more centers of chirality and exist as stereochemically isomeric forms.

The term “stereochemically isomeric forms” as used hereinbefore defines all the possible stereoisomeric forms which the compounds of Formulae I, Ia, and Ib, as illustrated herein, and the compounds of Tables 1A and 1B, and their N-oxides, addition salts, quaternary amines or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of the compounds of Formulae I, Ia, and Ib, as illustrated herein, and the compounds of Tables 1A and 1B, and their N-oxides, salts, solvates or quaternary amines substantially free, i.e. associated with less than 10%, preferably less than 5%, in particular less than 2% and most preferably less than 1% of the other isomers. In particular, stereogenic centers can have the R- or S-configuration; substituents on bivalent cyclic (partially) saturated radicals can have either the cis- or trans-configuration. Compounds encompassing double bonds can have an E or Z-stereochemistry at said double bond. Stereochemically isomeric forms of the compounds of Formulae I, Ia, and Ib, as illustrated herein, and the compounds of Tables 1A and 1B, are fully intended to be embraced within the scope of this invention.

“N-oxides” are meant to comprise the compounds of Formulae I, Ia, and Ib, as illustrated herein, and the compounds of Tables 1A and 1B, wherein one or several nitrogen atoms are oxidized to the so-called N-oxide.

Some of the compounds of Formulae I, Ia, and Ib, as illustrated herein, and the compounds of Tables 1A and 1B, can also exist in their tautomeric form. Such forms although not explicitly indicated in the above formula are intended to be included within the scope of the present invention.

In all compounds of the present invention, such as, for example, the compounds of Formulae I, Ia, and Ib, as illustrated herein, and the compounds of Tables 1A and 1B, reference to any bound hydrogen atom can also encompass a deuterium atom bound at the same position. Substitution of hydrogen atoms with deuterium atoms is conventional in the art. See, e.g., U.S. Pat. Nos. 5,149,820 & 7,317,039, which are incorporated by reference herein their entirety. Such deuteration sometimes results in a compound that is functionally indistinct from its hydrogenated counterpart, but occasionally results in a compound having beneficial changes in the properties relative to the non-deuterated form. For example, in certain instances, replacement of specific bound hydrogen atoms with deuterium atoms slows the catabolism of the deuterated compound, relative to the non-deuterated compound, such that the deuterated compound exhibits a longer half-life in the bodies of individuals administered such compounds. This particularly so when the catabolism of the hydrogenated compound is mediated by cytochrome P450 systems. See Kushner et al., Can. J. Physiol. Pharmacol. 77:79-88 (1999), which is incorporated by reference herein its entirety.

### 3. Pharmaceutical Compositions and Formulations

In another aspect, the present invention further provides a composition for use as a medicament or a pharmaceutical composition comprising one of the compounds of the present invention, such as, for example, the compounds of Formulae I, Ia, and Ib, as illustrated herein, and the compounds of Tables 1A and 1B, and a pharmaceutically-acceptable excipient. In some of such embodiments, the medicament or pharmaceutical composition comprises a therapeutically or prophylactically effective amount of at least one of the compounds of Formulae I, Ia, and Ib, as illustrated herein, and the compounds of Tables 1A and 1B.

In some of such embodiments, the composition or pharmaceutical composition is for use in treating cancer, systemic or chronic inflammation, rheumatoid arthritis, diabetes, obesity, T-cell mediated autoimmune disease, ischemia, and other complications associated with these diseases and disorders. In some of such embodiments, the composition or pharmaceutical composition is for use in treating cancer.

Typically, one of the compounds of the present invention, such as, for example, the compounds of Formulae I, Ia, and Ib, as illustrated herein, and the compounds of Tables 1A and 1B, can be effective at an amount of from about 0.01 μg/kg to about 100 mg/kg per day based on total body weight. The active ingredient can be administered at once, or can be divided into a number of smaller doses to be administered at predetermined intervals of time. The suitable dosage unit for each administration can be, e.g., from about 1 μg to about 2000 mg, preferably from about 5 μg to about 1000 mg. The pharmacology and toxicology of many of such other anticancer compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be applicable to the compounds of the present invention, such as, for example, the compounds of Formulae I, Ia, and Ib, as illustrated herein, and the compounds of Tables 1A and 1B.

It should be understood that the dosage ranges set forth above are exemplary only and are not intended to limit the scope of this invention. The therapeutically effective amount for individual compounds of the present invention, such as, for example, the compounds of Formulae I, Ia, and Ib, as illustrated herein, and the compounds of Tables 1A and 1B, can vary with factors including but not limited to the activity of the compound used, the stability of the compound used in the patient's body, the severity of the conditions to be alleviated, the total weight of the patient treated, the route of administration, the ease of absorption, distribution, and excretion of the compound by the body, the age and sensitivity of the patient to be treated, and the like, as will be apparent to a skilled artisan. The amount of administration can be adjusted as the various factors change over time.

In the pharmaceutical compositions, the compounds of the present invention, such as, for example, the compounds of Formulae I, Ia, and Ib, as illustrated herein, and the compounds of Tables 1A and 1B, can be in any pharmaceutically acceptable salt form, as described above.

For oral delivery, the compounds of the present invention, such as, for example, the compounds of Formulae I, Ia, and Ib, as illustrated herein, and the compounds of Tables 1A and 1B, can be incorporated into a formulation that includes pharmaceutically acceptable excipients or carriers such as binders, lubricants, disintegrating agents, and sweetening or flavoring agents, all known in the art. The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of a solution, suspension, syrup, chewing gum, wafer, elixir, and the like. If desired, conventional agents for modifying flavors, tastes, colors, and shapes of the special forms can also be included.

The compounds of the present invention, such as, for example, the compounds of Formulae I, Ia, and Ib, as illustrated herein, and the compounds of Tables 1A and 1B, can also be administered parenterally in the form of a solution or suspension, or in a lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. The parenteral formulations can be stored in any conventional containers such as vials and ampoules.

Routes of topical administration include dermal, nasal, bucal, mucosal, rectal, or vaginal applications. For topical administration, the compounds of the present invention, such as, for example, the compounds of Formulae I, Ia, and Ib, as illustrated herein, and the compounds of Tables 1A and 1B, can be formulated into lotions, creams, ointments, gels, powders, pastes, sprays, suspensions, drops and aerosols. Thus, one or more thickening agents, humectants, and stabilizing agents can be included in the formulations. A special form of topical administration is delivery by a transdermal patch. Methods for preparing transdermal patches that can be used with the compounds of the present invention, such as, for example, the compounds of Formulae I, Ia, and Ib, as illustrated herein, and the compounds of Tables 1A and 1B, are disclosed, e.g., in Brown, et al., Annual Review of Medicine, 39:221-229 (1988), which is incorporated herein by reference.

Subcutaneous implantation for sustained release of the compounds of the present invention, such as, for example, the compounds of Formulae I, Ia, and Ib, as illustrated herein, and the compounds of Tables 1A and 1B, can also be a suitable route of administration. This entails surgical procedures for implanting one or more of the compounds of the present invention, such as, for example, the compounds of Formulae I, Ia, and Ib, as illustrated herein, and the compounds of Tables 1A and 1B, in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the compounds of the present invention, such as, for example, the compounds of Formulae I, Ia, and Ib, as illustrated herein, and the compounds of Tables 1A and 1B. Hydrogels are generally known in the art. They are typically made by crosslinking high molecular weight biocompatible polymers into a network, which swells in water to form a gel-like material. Preferably, hydrogels are biodegradable or biosorbable. See, e.g., Phillips et al., J. Pharmaceut. Sci., 73:1718-1720 (1984).

The compounds of the present invention, such as, for example, the compounds of Formulae I, Ia, and Ib, as illustrated herein, and the compounds of Tables 1A and 1B, can also be conjugated, to a water soluble, non-immunogenic, non-peptidic, high molecular weight polymer to form a polymer conjugate. For example, one or more of the compounds of the present invention, such as, for example, the compounds of Formulae I, Ia, and Ib, as illustrated herein, and the compounds of Tables 1A and 1B, is covalently linked to polyethylene glycol to form a conjugate. Typically, such a conjugate exhibits improved solubility, stability, and reduced toxicity and immunogenicity. Thus, when administered to a patient, compounds of the present invention, such as, for example, the compounds of Formulae I, Ia, and Ib, as illustrated herein, and the compounds of Tables 1A and 1B, in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN) is being used to treat severe combined immunodeficiency disease (SCIDS). PEGylated L-asparaginase (ONCAPSPAR) is being used to treat acute lymphoblastic leukemia (ALL).

It is preferred that the covalent linkage between the polymer and one or more of the compounds of the present invention, such as, for example, the compounds of Formulae I, Ia, and Ib, as illustrated herein, and the compounds of Tables 1A and 1B, and/or the polymer itself is hydrolytically degradable under physiological conditions. Such conjugates can readily release the compounds of the present invention, such as, for example, the compounds of Formulae I, Ia, and Ib, as illustrated herein, and the compounds of Tables 1A and 1B, inside the body. Controlled release of the compounds of the present invention, such as, for example, the compounds of Formulae I, Ia, and Ib, as illustrated herein, and the compounds of Tables 1A and 1B, can also be achieved by incorporating one or more of the compounds of the present invention into microcapsules, nanocapsules, or hydrogels that are generally known in the art.

Liposomes can also be used as carriers for the compounds of the present invention, such as, for example, the compounds of Formulae I, Ia, and Ib, as illustrated herein, and the compounds of Tables 1A and 1B. Liposomes are micelles made of various lipids such as cholesterol, phospholipids, fatty acids, and derivatives thereof. Various modified lipids can also be used. Liposomes can reduce toxicity of the compounds of the present invention, and can increase their stability. Methods for preparing liposomal suspensions containing active ingredients therein are generally known in the art, and, thus, can be used with the compounds of the present invention. See, e.g., U.S. Pat. No. 4,522,811; Prescott, Ed., Methods in Cell Biology, Volume XIV, Academic Press, New York, N.Y. (1976).

### 4. Therapeutic Methods

Without wishing to be bound by theory, it is believed that metabolites of compounds of the present invention have Nampt inhibiting properties. For example, it is believed that compounds having a structure according to Formula I may be metabolized to form compounds having a structure according to Parent Compound I (See Section 6 below). Likewise, it is believed that compounds having a structure according to Formula Ia may be metabolized to form compounds having a structure according to Parent Compound Ia. For example, it is believed that Example Compound Nos. 34 through 60 may metabolize to form Parent Compound A, 1-{4-[2-(Biphenyl-2-yl)ethoxy]phenyl}-3-(pyridin-3-ylmethyl)urea. For example, it is believed that Example Compound No. 35 may metabolize to form Parent Compound A, benzoic acid, and formaldehyde.

It is also believed that compounds having a structure according to Formula Ib may be metabolized to form compounds having a structure according to Parent Compound Ib. For example, it is believed that Example Compound Nos. 1 through 33 may metabolize to form Parent Compound B.

Compounds encompassed by Parent Compound I, Parent Compound Ia, and Parent Compound Ib are disclosed in International Patent Application No. PCT/US 11/26752, filed Mar. 1, 2011, and published as WO/2011/109441, the entire contents of which are incorporated herein in its entirety. Specifically, Parent Compound A and Parent Compound B are disclosed in WO/2011/109441.

It should be understood that the same compound that may be a metabolite of a compound having a structure according to Formula I may also be used as a building block for such compound having a structure according to Formula I. For example, Parent Compound A may be a metabolite of Example Compound Nos. 34 through 60 and Parent Compound A may also be used as a building block for making Example Compound Nos 34 through 60, as discussed in Section 6 below.

The present invention provides, among other things, therapeutic methods for treating diseases and disorders that will respond to therapy with a Nampt inhibitor. Consequently, the present invention provides therapeutic methods for treating cancer, systemic or chronic inflammation, rheumatoid arthritis, diabetes, obesity, T-cell mediated autoimmune disease, ischemia, and other complications associated with these diseases and disorders. These therapeutic methods involve treating a patient (either a human or another animal) in need of such treatment, with a therapeutically effective amount of one or more of the compounds of the present invention, such as, for example, the compounds of Formulae I, Ia, and Ib, as illustrated herein, and the compounds of Tables 1A and 1B, or a pharmaceutical composition comprising a therapeutically effective amount of one or more of the compounds of the present invention.

Additionally, the present invention provides the use of the compounds of the present invention, such as, for example, the compounds of Formulae I, Ia, and Ib, as illustrated herein, and the compounds of Tables 1A and 1B, or a pharmaceutical composition comprising a therapeutically effective amount of one or more of the compounds of the present invention, for the manufacture of a medicament useful for human therapy.

In some of such embodiments, the therapy comprises therapy for the treatment of cancer, systemic or chronic inflammation, rheumatoid arthritis, diabetes, obesity, T-cell mediated autoimmune disease, ischemia, and other complications associated with these diseases and disorders, in a human patient.

In some of such embodiments, the therapy comprises therapy for the delaying the onset of, or reducing the symptoms of, cancer, systemic or chronic inflammation, rheumatoid arthritis, diabetes, obesity, T-cell mediated autoimmune disease, ischemia, and other complications associated with these diseases and disorders, in a human patient.

As used herein, the phrase “treating . . . with . . . a compound” means either administering one or more of the compounds of the present invention, such as, for example, the compounds of Formulae I, Ia, and Ib, as illustrated herein, and the compounds of Tables 1A and 1B, or a pharmaceutical composition comprising one or more of the compounds of the present invention, directly to an animal, or administering to an animal another agent to cause the presence or formation of one or more of the compounds of the present invention inside the animal.

Preferably, the methods of the present invention comprise administering to cells in vitro or to a warm-blood animal, particularly mammal, and more particularly a human, a pharmaceutical composition comprising an effective amount of one or more of the compounds of the present invention, such as, for example, the compounds of Formulae I, Ia, and Ib, as illustrated herein, and the compounds of Tables 1A and 1B, or another agent to cause the presence or formation of one or more of the compounds of the present invention inside the cells or the animal.

As would be appreciated by the skilled artisan, one or more of the compounds of the present invention, such as, for example, the compounds of Formulae I, Ia, and Ib, as illustrated herein, and the compounds of Tables 1A and 1B, can be administered in one dose at one time, or can be divided into a number of smaller doses to be administered at predetermined intervals of time. The suitable dosage unit for each administration can be determined based on the effective daily amount and the pharmacokinetics of the compounds.

a. Treating Cancer:

In particular embodiments, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient. Support for treating cancer with a Nampt inhibitor may be found in WO/2011/109441, among other places.

In some embodiments, the patient is a human patient.

In some embodiments, the method comprises identifying a patient in need of such treatment. A patient having cancer can be identified by conventional diagnostic techniques known in the art, as well as by those methods discussed herein below.

WO/2011/109441 discloses that cancers that express low levels of the Nampt enzyme may be more susceptible to treatment with a Nampt inhibitor, than a cancer that expresses high levels of the Nampt enzyme. Accordingly, in one aspect, the present invention provides methods of treating cancer, comprising first identifying a cancer exhibiting a low level of Nampt expression. The methods further comprise administering to a patient having a cancer exhibiting low levels of Nampt expression, a therapeutically effective dose of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B.

As disclosed in WO/2011/109441, it is believed that inhibition of Nampt activity would be effective in treating a wide range of cancers. Consequently, the present invention provides methods of treating a wide range of cancers by administering therapeutically effective amounts of one or more of the compounds of the present invention. Specifically, it has been discovered that cancer cell types corresponding to colon, prostate, breast, NSCLC, sarcoma, pancreatic, SCLC, gastric, myeloma, ovarian, lymphoma, and glioma cancers are killed by Nampt inhibiting compounds.

Thus, in one embodiment, the present invention provides a method of treating colon cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating prostate cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating breast cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating non-small-cell lung cancer (NSCLC), comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating sarcoma cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating pancreatic cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating SCLC cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating gastric cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating myeloma cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating ovarian cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating lymphoma cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating glioma cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

As used herein, the term “cancer” has its conventional meaning in the art. Cancer includes any condition of the animal or human body characterized by abnormal cellular proliferation. The cancers to be treated comprise a group of diseases characterized by the uncontrolled growth and spread of abnormal cells. Compounds of the present invention have been shown to be effective in a variety of standard cancer models, and are thus thought to have utility in treating a broad range of cancers. However, preferred methods of the invention involve treating cancers that have been found to respond favorably to treatment with Nampt inhibitors. Further, “treating cancer” should be understood as encompassing treating a patient who is at any one of the several stages of cancer, including diagnosed but as yet asymptomatic cancer.

Specific cancers that can be treated by the methods of the invention are those cancers that respond favorably to treatment with a Nampt inhibitor. Such cancers include, but are not limited to, Hodgkin's disease, non-Hodgkin's lymphoma, acute lymphocytic leukemia, chronic lymphocytic leukemia, acute myeloid leukemia, mantle-cell lymphoma, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

WO/2011/109441 discloses methods of identifying cancers most likely to be susceptible to treatment with Nampt inhibitors. Accordingly, embodiments of the present invention include a method of identifying a cancer that is likely susceptible to treatement with a compound of the present invention, such as, for example, a compound of Formulae I, Ia, and Ib, as illustrated herein, and a compound of Tables 1A and 1B. The method comprises obtaining a biopsy sample of said cancer, determining the expression level of enzymes in pathways for NAD biosynthesis (e.g. tryptophan, kynurenine pathway, nicotinic acid (NA) salvage pathway, nicotinamide riboside pathway), relative to a non-cancerous control tissue, wherein, if the expression level of enzymes in such pathways (e.g. Naprt1, Qprt, NRK-1) is reduced, relative to a non-cancerous control tissue, the cancer is identified as likely susceptible to treatment with a compound of the present invention.

In some of such embodiments, the methods of determining the expression level of the Naprt1 gene involve either determining levels of expression of the Naprt1-encoding transcript (i.e., Naprt1-encoding mRNA), or determining levels of expression of the Naprt1 protein itself. For these embodiments, any acceptable means of determining expression levels of either the Naprt1-encoding transcript, or the Naprt1 protein itself, can be utilized, and such acceptable means are well within the skill level of the artisan versed in determining expression levels of eukaryotic genes. Such acceptable means can include, for example, quantitative PCR (qPCR) to measure levels of Naprt1-encoding transcript, or ELISAs to measure levels of expressed Naprt1 protein. The specific methods involved in determining the expression of particular eukaryotic genes are well known in the art.

Additionally, embodiments of the present invention include a method of treating cancer, wherein cells of the cancer exhibit low levels of Naprt1 expression. Thus, in one embodiment, the present invention provides a method of treating a cancer that exhibit low levels of Naprt1 expression, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

WO/2011/109441 discloses that Naprt1 expression was least in brain cancers, lung cancers, lymphoma, myeloma and osteosarcoma out of cell lines screened for Naprt1 expression. Further, glioblastoma and sarcoma cell lines that are reported to be resistant to nicotinic acid (NA) rescue have been found to have reduced Naprt1 expression (Watson, et al. Mol. Cell. Biol. 29(21):5872-88 (2009)).

Thus, in one embodiment, the present invention provides a method of treating brain cancer, such as glioblastoma, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating lung cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

Thus, in one embodiment, the present invention provides a method of treating osteosarcoma cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, to a patient.

WO/2011/109441 discloses methods of limiting toxicity of Nampt inhibiting compounds by administering nicotinic acid (NA).

Those cancers with reduced or absent levels of Naprt1 expression should be more susceptible to treatment with the Nampt inhibitors of the present invention, administration of NA to patients having such cancers could prevent toxicity in other tissues associated with Nampt inhibition. WO/2011/109441 discloses information to support this concept

Accordingly, in some embodiments, the methods of treating cancer disclosed herein further comprise administering nicotinic acid, or a compound capable of forming nicotinic acid in vivo, to the patient in addition to administering a compound of the present invention, such as, for example, a compound of Formulae I, Ia, and Ib, as illustrated herein, and a compound of Tables 1A and 1B. In some of such embodiments, the compound of the present invention is able to be administered at dose that exceeds the maximum tolerated dose for that particular compound of the present invention as determined for mono-therapy.

In some of such embodiments, administering NA may include administering NA prior to administering one or more of the compounds of the present invention, co-administering NA with one or more of the compounds of the present invention, or first treating the patient with one or more of the compounds of the present invention, followed by thereafter administering NA.

b. Treating Systemic or Chronic Inflammation

Support for treating inflammation with a Nampt inhibitor may be found in WO/2011/109441, among other places. It is believed that inhibition of Nampt activity would be effective in treating systemic or chronic inflammation resulting from a wide range of causes. Consequently, the present invention provides methods of treating systemic or chronic inflammation by administering therapeutically effective amounts of one or more of the compounds of the present invention.

c. Treating Rheumatoid Arthritis

Support for treating rheumatoid arthritis (“RA”) with a Nampt inhibitor may be found in WO/2011/109441, among other places. It is believed that inhibition of Nampt activity would be effective in treating RA. Consequently, the present invention provides methods of treating RA by administering therapeutically effective amounts of one or more of the compounds of the present invention, either alone, or in combination with a PARP inhibitor.

d. Treating Obesity and Diabetes

Support for treating obesity and diabetes with a Nampt inhibitor may be found in WO/2011/109441, among other places. It is believed that inhibition of Nampt activity would be effective in treating obesity and diabetes, and other complications associated with these, and other, metabolic diseases and disorders. Consequently, the present invention provides methods of treating obesity and diabetes, and other complications associated with these, and other, metabolic diseases and disorders, by administering therapeutically effective amounts of one or more of the compounds of the present invention.

e. Treating T-Cell Mediated Autoimmune Disease

Support for treating T-cell mediated autoimmune disease with a Nampt inhibitor may be found in WO/2011/109441, among other places. It is believed that inhibition of Nampt activity would be effective in treating T-cell mediated autoimmune disease, and other complications associated with diseases and disorders. Consequently, the present invention provides methods of treating T-cell mediated autoimmune disease, and other complications associated with these diseases and disorders, by administering therapeutically effective amounts of one or more of the compounds of the present invention.

f. Treating Ischemia

Support for treating ischemia with a Nampt inhibitor may be found in WO/2011/109441, among other places. It is believed that inhibition of Nampt activity would be effective in treating ischemia and other complications associated with this condition. Consequently, the present invention provides methods of treating ischemia and other complications associated with this condition, by administering therapeutically effective amounts of one or more of the compounds of the present invention, either alone, or in combination with a PARP inhibitor.

### 5. Combination Therapy

In an additional aspect, the present invention also provides methods for combination therapy for treating cancer, systemic or chronic inflammation, rheumatoid arthritis, diabetes, obesity, T-cell mediated autoimmune disease, ischemia, and other complications associated with these diseases and disorders, by treating a patient in need thereof, with a therapeutically effective amount of one of the compounds of the present invention together with a therapeutically effective amount of one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders.

In some embodiments, the present invention provides methods for combination therapy for treating cancer by treating a patient (either a human or another animal) in need of the treatment with one of the compounds of the present invention together with one or more other anti-cancer therapies. Such other anti-cancer therapies include traditional chemotherapy agents, targeted agents, radiation therapy, surgery, hormone therapy, immune adjuvants, etc. In the combination therapy, one of the compounds of the present invention, such as, for example, a compound of Formulae I, Ia, and Ib, as illustrated herein, and a compound of Tables 1A and 1B, can be administered separately from, or together with the one or more other anti-cancer therapies.

Specifically, WO/2011/109441, among others, discloses that Nampt inhibition has been shown to sensitize cells to the effects of various chemotherapeutic or cytotoxic agents. Without wishing to be bound by theory, it is believed that sub-lethal NAD⁺ drops render cells vulnerable to other cytotoxic agents, and particularly to compounds which activate the DNA repair enzyme poly(ADP-ribose) polymerase (PARP), since PARP requires NAD⁺ as a substrate and consumes NAD⁺ during its enzymatic action.

Accordingly, in some embodiments, the present invention provides the methods of treating cancer disclosed herein further comprise administering a therapeutically-effective amount of a PARP activator to the patient in addition to administering a compound of the present invention, such as, for example, a compound of Formulae I, Ia, and Ib, as illustrated herein, and a compound of Tables 1A and 1B.

Additionally, in some of such embodiments, the cells of the cancer have functional homologous recombination (HR) systems. Also, in some of such embodiments, the methods further comprise identifying the cells of the cancer as having functional HR systems. Methods of performing such identification are known in the art. Furthermore, in addition to a PARP activator, in some embodiments, the methods of treating cancer disclosed herein further comprise administering a therapeutically effective amount of a non-DNA damaging agent to the patient, wherein the non-DNA damaging agent is not a PARP activator and not a compound of the present invention. For example, where the cancer has functional HR systems for repairing DNA damage, then an additional chemotherapeutic could be administered that does not rely on DNA damage for efficacy. Chemotherapeutics the do not damage DNA are known in the art.

Agents or treatments that may be capable of activating the PARP enzyme include but are not limited to: alkylating agents (methyl methane sulfonate (MMS), N-methyl-N′nitro-N-nitrosoguanidine (MNNG), Nitrosoureas (N-methyl-N-nitrosourea (MNU), streptozotocin, carmustine, lomustine), Nitrogen mustards (melphalan, cyclophosphamide, uramustine, ifosfamide, clorambucil, mechlorethamine), alkyl sulfonates (busulfan), platins (cisplatin, oxaliplatin, carboplatin, nedaplatin, satraplatin, triplatin tetranitrate), non-classical DNA alkylating agents (temozolomide, dacarbazine, mitozolamide, procarbazine, altretamine)), radiation (X-rays, gamma rays, charged particles, UV, systemic or targeted radioisotope therapy), and other DNA damaging agents such as: topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

It is further believed that tumors or tumor cell lines treated with compounds that directly or indirectly inhibit the enzyme thymidylate synthase (TS) can also be more susceptible to Nampt inhibitors, such as compounds of the present invention.

Accordingly, in some embodiments, the present invention provides the methods of treating cancer disclosed herein further comprise administering a therapeutically-effective amount of a thymidylate synthase inhibitor to the patient in addition to administering a compound of the present invention, such as, for example, a compound of Formulae I, Ia, and Ib, as illustrated herein, and a compound of Tables 1A and 1B.

In some embodiments, the thymidylate synthase inhibitor directly or indirectly inhibits thymidylate synthase. Thymidylate synthase inhibitors include 5-FU, raltitrexed, pemetrexed, and other TS inhibitors developed over the past decades.

It is further believed that agents that promote aberrant uracil incorporation into DNA can also make subjects being administered such agents more susceptible to Nampt inhibitors, such as compounds of the present invention. Any inhibitor of thymidylate synthase (TS) would cause uracil incorporation into DNA. Other agents, such as inhibitors of dihydrofolate reductase (e.g. methotrexate) have also been shown to cause uracil to aberrantly incorporate into DNA.

Accordingly, in some embodiments, the present invention provides the methods of treating cancer disclosed herein further comprise administering a therapeutically-effective amount of agents that promote aberrant uracil incorporation into DNA, to the patient in addition to administering a compound of the present invention, such as, for example, a compound of Formulae I, Ia, and Ib, as illustrated herein, and a compound of Tables 1A and 1B.

In view of the above, some embodiments of the present invention comprises the use of the compounds of the present invention with a second chemotherapeutic agent that has been discovered to work synergistically with one or more of the compounds of the present invention, such as compounds or treatments that activate PARP, induce DNA damage, inhibit TS, and/or promote aberrant uracil incorporation into DNA, or inhibit proteasomes or specific kinases.

In certain embodiments of this aspect of the invention, the second chemotherapeutic agent is selected from, at least, methyl methanesulfonate (MMS), mechlorethamine, streptozotocin, 5-fluorouracil (5-FU), raltitrexed, methotrexate, bortezomib, PI-103, and dasatinib.

WO/2011/109441 discloses support for the suggestion that the drug combination of Nampt inhibitor plus a PARP inhibitor would be antagonistic in normal cells, but synergistic in cells that do not have functional HR systems or that have reduced functioning HR systems, such as cells that have lost BRCA tumor suppressor function.

Accordingly, in some embodiments, the present invention provides the methods of treating cancer disclosed herein further comprise administering a therapeutically-effective amount of a PARP inhibitor to the patient in addition to administering a compound of the present invention, such as, for example, a compound of Formulae I, Ia, and Ib, as illustrated herein, and a compound of Tables 1A and 1B.

In some of such embodiments, the cells of the cancer do not have functional homologous recombination (HR) systems. In some of such embodiments, the methods of treating cancer further comprise identifying the cells of the cancer as not having functional HR systems. Methods of performing such identification are known in the art.

In some of such embodiments, the PARP inhibitor is olaparib, AG014699/PF-01367338, INO-1001, ABT-888, Iniparib, BSI-410, CEP-9722, MK4827, or E7016.

In some of such embodiments, the methods further comprise administering a therapeutically effective amount of a DNA damaging agent to the patient, wherein the DNA damaging agent is other than a PARP inhibitor. DNA damaging agents are known in the art and include topoisomerase inhibitors (camptothecin, beta-lapachone, irinotecan, etoposide), anthracyclines (doxorubicin, daunorubicin, epirubicin, idarubicin, valrubicin, mitoxantrone), reactive oxygen generators (menadione, peroxynitrite), and anti-metabolites (5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, floxuridine).

WO/2011/109441 discloses synergistic combinations of Nampt inhibitors and standards of care in particular cancer types. Cancer cell lines used in these studies represented cancer types found to be sensitive to Nampt inhibition [e.g. non-Hodgkins lymphoma, multiple myeloma, glioma, non-small cell lung carcinoma (NSCLC), small cell lung carcinoma (SCLC), ovarian cancer and colorectal cancer]. Standards of care in these cancer types tested in synergy experiments included: 4-HC (the pre-activated form of cyclophosphamide), doxorubicin, vincristine, prednisolone, dexamethasone, melphalan, thalidomide, bortezomib, temozolomide, cisplatin, paclitaxel, gefitinib, 5-FU, oxaliplatin, irinotecan, and etoposide. Synergistic cytotoxicity was found when nampt inhibitors were combined with 4HC in small-cell lung cancer (SCLC) and glioma, temozolomide in glioma, and 5-FU in colon cancer.

Another specific example of an active agent with which the compounds of the present invention can be co-administered is the immune adjuvant L-1-methyl tryptophan (L-1MT). In studies of co-administration of L-1MT with another inhibitor of Nampt (i.e., AP0866 [also known as FK866 or WK175]), the combination was shown to provide an additive inhibitory effect on tumor growth of murine gastric and bladder tumors in immune-competent mice (Yang et al. Exp. Biol. Med. 235:869-76 (2010)).

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of temozolomide, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of 4HC, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of 5-FU, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of L-1MT, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of methyl methanesulfonate (MMS), to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of mechlorethamine, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of streptozotocin, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of raltitrexed, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of methotrexate, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of bortezomib, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of PI-103, to a patient.

Thus, in one embodiment, the present invention provides a method of treating cancer, comprising administering a therapeutically effective amount of one or more compounds of the present invention, such as, for example, the compounds of Formulae I, Ia, and Ib, as illustrated herein, and the compounds of Tables 1A and 1B, or a pharmaceutical composition comprising one or more compounds of the present invention, such as, for example, the compounds of Formulae I, Ia, and Ib, as illustrated herein, and the compounds of Tables 1A and 1B, and administering a therapeutically-effective amount of dasatinib, to a patient.

In the case of combination therapy, a therapeutically effective amount of one or more other therapeutically effective compounds can be administered in a separate pharmaceutical composition, or alternatively included in the same pharmaceutical composition of the present invention which contains one of the compounds of the present invention. One or more of the compounds of the present invention can be administered together in the same formulation with the one or more other compounds that have been shown to be effective in the treatment of cancer, systemic or chronic inflammation, rheumatoid arthritis, diabetes, obesity, T-cell mediated autoimmune disease, ischemia, and other complications associated with these diseases and disorders, in the same formulation or dosage form. Thus, the present invention also provides pharmaceutical compositions or medicaments for combination therapy, comprising an effective amount of one or more of the compounds of the present invention, and an effective amount of at least one other compound that has been shown to be effective in the treatment of cancer, systemic or chronic inflammation, rheumatoid arthritis, diabetes, obesity, T-cell mediated autoimmune disease, ischemia, and other complications associated with these diseases and disorders.

The compounds of the present invention can also be administered in combination with another active agent that synergistically treats or prevents the same symptoms or is effective for another disease or symptom in the patient being treated, so long as the other active agent does not interfere with, or adversely affect, the effects of the compounds of the present invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, immune adjuvants, and the like.

### 6. Methods of Making the Compounds of the Present Invention

In an additional aspect, the present invention provides methods of the making the compounds of the present invention. Embodiments of methods of making the compounds of the present invention are provided in Procedures A through F below.

In some embodiments, a method of making a compound, comprises reacting a compound having a structure according to Parent Compound I (as defined below) with a desired Prodrug Moiety (as defined below) under suitable conditions to yield a compound having a structure according to Formula I.

In some embodiments, a method of making a compound, comprises reacting a compound having a structure according to Parent Compound Ia (as defined below) with a desired Prodrug Moiety (as defined below) under suitable conditions to yield a compound having a structure according to Formula Ia.

In some embodiments, a method of making a compound, comprises reacting a compound having a structure according to Parent Compound Ib (as defined below) with a desired Prodrug Moiety (as defined below) under suitable conditions to yield a compound having a structure according to Formula Ib.

### Synthetic Schemes

Procedure A

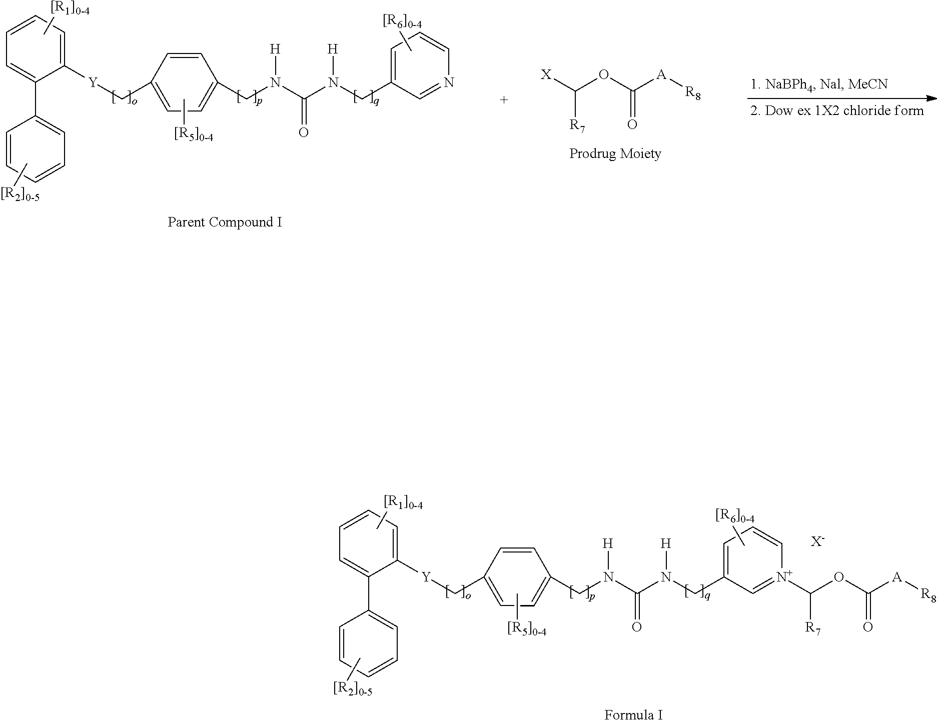

R₁, R₂, R₅, R₆, Y, o, p, and q of Parent Compound I are as defined for Formula I above. Methods of making compounds encompassed by Parent Compound I are disclosed in WO/2011/109441. In particular, methods of making Parent Compound A and Parent Compound B are disclosed in WO/2011/109441

The appropriate Parent Compound I (1 equiv), NaBPh₄ (1.2 equiv), NaI (1.2 equiv) in MeCN (0.1 M) is added the appropriate Prodrug Moiety (1.2 equiv) (X=Chloro or Bromo, A, R₇, and R₈ as defined in Formula I) and the solution is stirred at 60° C. overnight.

It should be understood that X as defined for the Prodrug Moiety is different from the X as defined for each of Formulae I, Ia, and Ib, even though X for each of Formulae I, Ia, and Ib encompasses Chloro and Bromo. The reaction mixture is filtered and the filtrate concentrated and dissolved in a small amount of 1:1 IPA:MeCN and passed over a column of Dowex (1×2, Cl— form, strongly basic; Sigma-Aldrich #44290). On large scale the reaction mixture is dilluted with IPA (volume approximately equal to the volume of MeCH) before the initial filtration. Alternatively the crude product is stirred with Dowex in 1:1 IPA:MeCN for 30 min-2 h. and filtered over a plug of Dowex. The column eluent is concentrated and purified by HPLC (0-20% MeOH/DCM). Procedure is adapted from J. Med. Chem. 1994, 37, 4423-4429, the entire contents of which are incorporated herein by reference.

Compounds can be recrystallized as follows:

To a large vial the compound of Formula I is added stirring in a minimal amount of EtOAc (Igram approx 20 mL). To this solution DCM is slowly added (approx 2-5 mL). The reaction is sealed and heated, off white solid crashes out of solution. The solid is collected via vacuum filtration and placed in another vial. DCM is added (15-20 mL) and the solution is warmed, MeOH is slowly added until complete dissolution occurs. The solution is placed at room temperature and solids are collected after sitting overnight allowing crystals to grow.

It should be understood that if the desired product of Procedure A is a compound according to Formula Ia, then a compound having a structure according to Parent Compound Ia could be used, wherein R₁, R₂, R₅, R₆, Y, u, O, p, and q are as defined for Formula Ia.

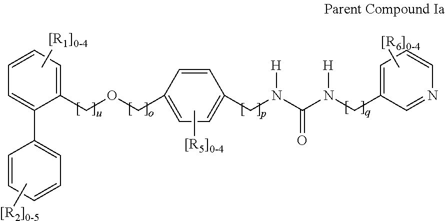

It should be understood that if the desired product of Procedure A is a compound according to Formula Ib, then a compound having a structure according to Parent Compound Ib could be used, wherein R₁, R₂, R₃, R₄, R₅, R₆, O, p, and q are as defined for Formula Ib.

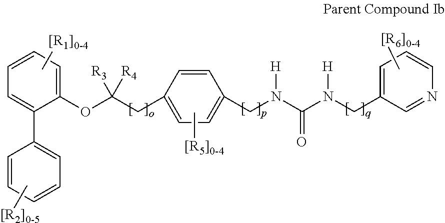

It should be understood that Example Compound Nos. 1 through 33 were made from Parent Compound B.

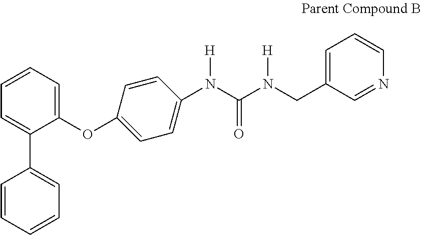 [SMILES: O=C(NCc1cccnc1)Nc1ccc(Oc2ccccc2-c2ccccc2)cc1]

It should be understood that Example Compound Nos. 34 through 60 were made from Parent Compound A.

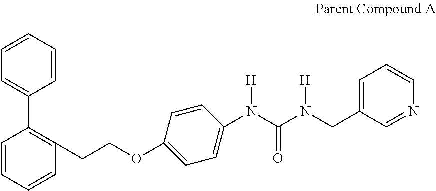 [SMILES: O=C(NCc1cccnc1)Nc1ccc(OCCc2ccccc2-c2ccccc2)cc1]

Procedures B through F disclose methods of making particular Prodrug Moieties for use with Procedure A. In Procedures B through F, if the R₇ moiety is not explicitly identified, then R₇ is hydro.

Procedure B

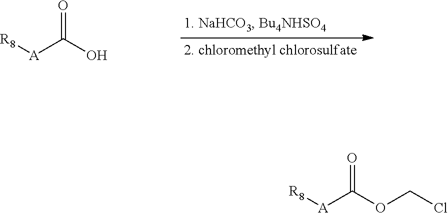

To the appropriate carboxylic acid (1 equiv) and Bu₄NHSO₄ (0.2 equiv) in DCM (0.2 M) at 0° C. is added sat. NaHCO₃ (5 equiv) and the solution is stirred at 0° C. for 15 min. Chloromethyl chlorosulfate (1.5 equiv) is added and the solution is allowed to warm gradually to rt. After 3-24 h the layers are separated, the organic layer dried with Na₂SO₄ and concentrated. The resulting chloromethyl ester is carried on without further purification.

Procedure C

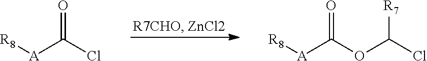

The appropriate acid chloride (1 equiv) is added to the appropriate aldehyde “R₇C(═O)H” (1 equiv) and ZnCl₂ (˜0.01 equiv) at −78° C. (0° C. in the case of PhCOC1) and the reaction mixture allowed to warm gradually to rt overnight. The solution is concentrated and used without further purification.

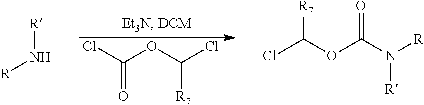

The appropriate chloroformate (1.2 equiv) is added slowly to the appropriate amine or ammonium salt (1 equiv) and Et₃N (3 equiv) in DCM (0.2 M). The reaction is stirred at rt for 2-3 h. A small amount of 10% HCl is added, the solution is passed through a phase separator column, and the solution is concentrated. Alternatively on larger scale, the layers are separated in a separatory funnel, the organic layer dried with Na₂SO₄ and concentrated. The resulting carbamate is used without further purification. In Procedure D, R and R′ with the linking Nitrogen form “A-R₈” as defined Formula I.

Note: for primary amines the entire reaction should be done at 0° C.

Procedure E

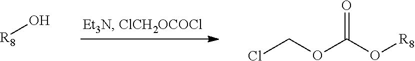

To chloromethyl chloroformate (1.0 equiv) in DCM (0.2 M) is added the appropriate alcohol (1.0 equiv) and Et₃N (1.0 equiv) at −10° C. The reaction is stirred at −10° C. to rt for 2-3 h. A small amount of sat. NaHCO₃ is added, the solution is passed through a phase separator column, and the solution is concentrated. Alternatively on larger scale, the layers are separated in a separatory funnel, the organic layer dried with Na₂SO₄ and concentrated. The resulting carbonate is used without further purification.

Procedure F

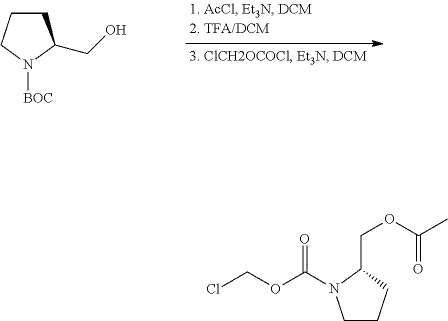

To N-boc-prolinol (1 equiv) and Et₃N (3 equiv) in DCM (0.2 M) at 0° C. is added AcCl (1.2 equiv). The solution is stirred at 0° C. to rt over 1 h, diluted with DCM, washed with 10% HCl, dried with Na₂SO₄ and concentrated. The resulting acetate is taken up in DCM (0.5 M) and TFA (˜2 mL/mmol) is added. The solution is stirred at rt. for 1-2 h. and concentrated. The resulting prolinol acetate is carried on according to the general procedure for making chloromethyl carbamates.

Exemplary compounds of the present invention are shown in Table 1. Table 1 is separated into an “A” and “B”. The “A” table shows the structure and name for a particular example compound. Compound names were generated using ACD Labs IUPAC nomenclature software version 12.00 (Toronto, Ontario, Canada).

The “B” table shows the Nuclear Magnetic Resonance (“NMR”) data, molecular weight calculated and found using High Resolution Mass Spectrometry (“HRMS”), and also lists the Synthetic Procedures used to make the particular example compound. In some instances, the Synthetic Procedure listed is similar to the procedure actually used to make a particular example compound, rather than the actual procedure used. Each of the example compounds were synthesized using commercially available starting materials that are well known in the art.

### Example Compounds

TABLE 1A

| Ex. | | |
|---|---|---|
| No. | Structure | IUPAC Name |
| 1 | 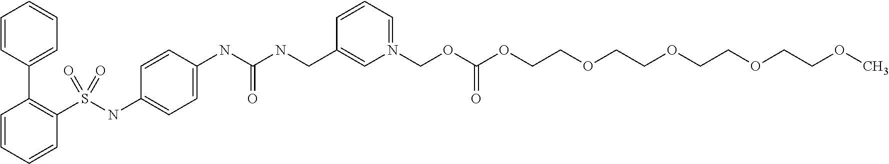 | 2-[2-[2-(2-methoxyethoxy)ethoxy]ethoxy]ethyl [3-[[[4-[(2- phenylphenyl)sulfonylamino]phenyl]carbamoylamino] methyl]-1-pyridyl]methyl carbonate |
| 2 | 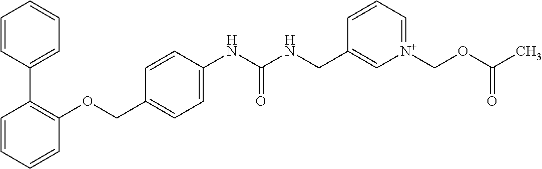 [SMILES: CC(=O)OC[n+]1cccc(CNC(=O)Nc2ccc(COc3ccccc3-c3ccccc3)cc2)c1] | l-[(Acetyloxy)methyl]-3-{[({4-[(biphenyl-2-yloxy)methyl] phenyl}carbamoyl)amino]methyl}pyridinium |
| 3 | 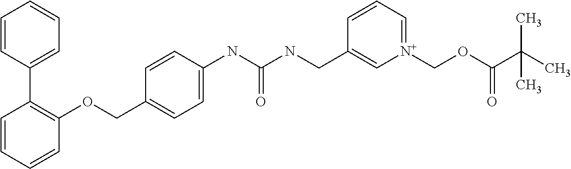 [SMILES: CC(C)(C)C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{[(2,2-dimethylpropanoyl)oxy]methyl} pyridinium |
| 4 | 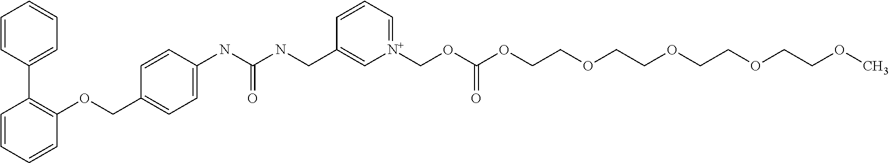 [SMILES: COCCOCCOCCOCCOC(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-(3-oxo-2,4,7,10,13,16-hexaoxaheptadec- 1-yl)pyridinium |
| 5 | 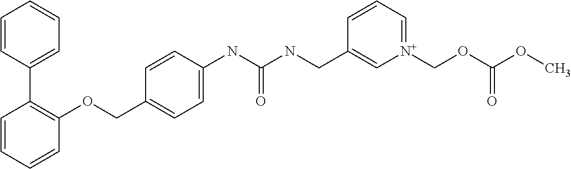 [SMILES: COC(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{[(methoxycarbonyl)oxy]methyl} pyridinium |
| 6 | 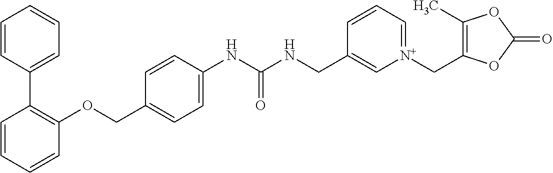 [SMILES: Cc1oc(=O)oc1C[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[(5-methyl-2-oxo-1,3-dioxol-4-yl) methyl]pyridinium |
| 7 | 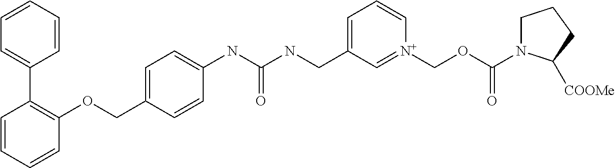 [SMILES: COC(=O)[C@@H]1CCCN1C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[({[(2S)-2-(methoxycarbonyl) pyrrolidin-1-yl]carbonyl}oxy)methyl]pyridinium |
| 8 | 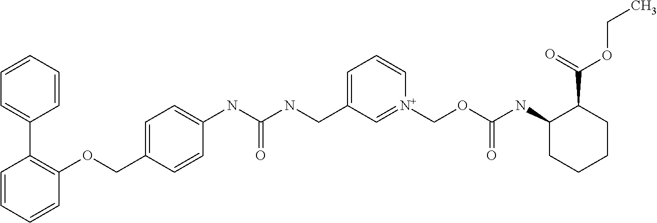 [SMILES: CCOC(=O)[C@H]1CCCC[C@H]1NC(=O)OC[n+]1cccc(CNC(=O)Nc2ccc(COc3ccccc3-c3ccccc3)cc2)c1] | rel-3-{[({4-[(Biphenyl-2-yloxy)methyl]phenyl}carbamoyl) amino]methyl}-1-[({[(lR,2S)-2-(ethoxycarbonyl) cyclohexyl]carbamoyl}oxy)methyl]pyridinium |
| 9 | 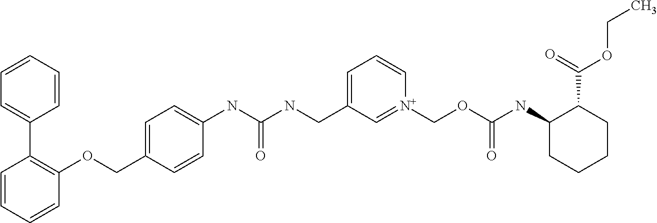 [SMILES: CCOC(=O)[C@@H]1CCCC[C@H]1NC(=O)OC[n+]1cccc(CNC(=O)Nc2ccc(COc3ccccc3-c3ccccc3)cc2)c1] | rel-3-{[({4-[(Biphenyl-2-yloxy)methyl]phenyl}carbamoyl) amino]methyl}-1-[({[(1R,2R)-2-(ethoxycarbonyl) cyclohexyl]carbamoyl}oxy)methyl]pyridinium |
| 10 | 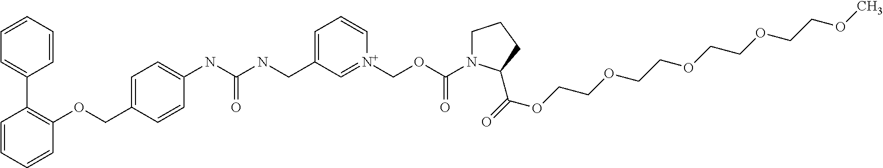 [SMILES: COCCOCCOCCOCCOC(=O)[C@@H]1CCCN1C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[({[(2S)-2-(2,5,8,11,14-pentaoxapenta- decan-1-oyl)pyrrolidin-1-yl]carbonyl}oxy)methyl] pyridinium |
| 11 | 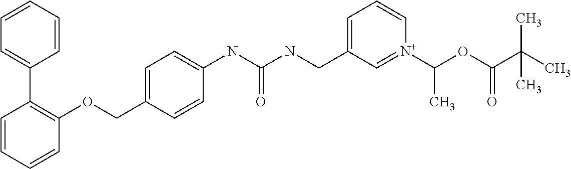 [SMILES: CC(OC(=O)C(C)(C)C)[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{1-[(2,2-dimethylpropanoyl)oxy] ethyl}pyridinium |
| 12 | 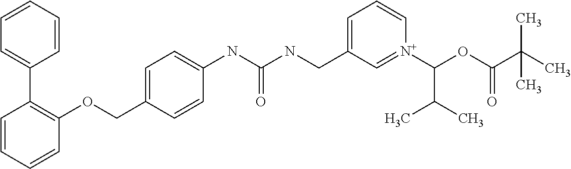 [SMILES: CC(C)C(OC(=O)C(C)(C)C)[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{1-[(2,2-dimethylpropanoyl)oxy]-2- methylpropyl}pyridinium |
| 13 | 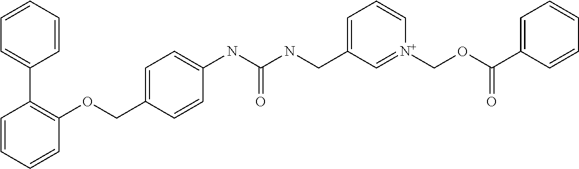 [SMILES: O=C(NCc1ccc[n+](COC(=O)c2ccccc2)c1)Nc1ccc(COc2ccccc2-c2ccccc2)cc1] | 1-[(Benzoyloxy)methyl]-3-{[({4-[(biphenyl-2-yloxy) methyl]phenyl}carbamoyl)amino]methyl}pyridinium |
| 14 | 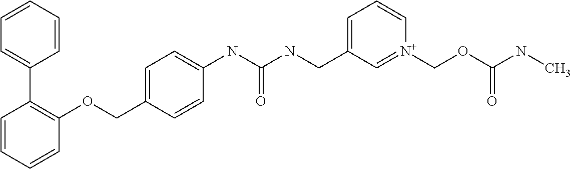 [SMILES: CNC(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{[(methylcarbamoyl)oxy]methyl} pyridinium |
| 15 | 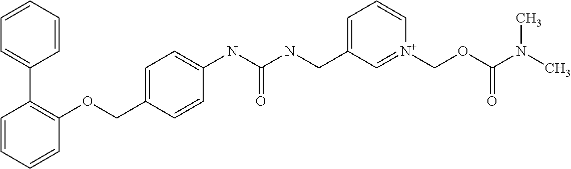 [SMILES: CN(C)C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{[(dimethylcarbamoyl)oxy] methyl}pyridinium |
| 16 | 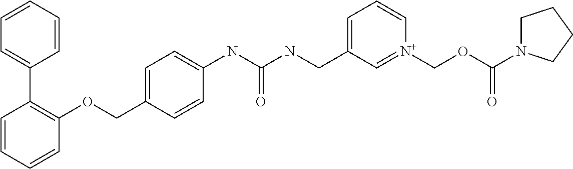 [SMILES: O=C(NCc1ccc[n+](COC(=O)N2CCCC2)c1)Nc1ccc(COc2ccccc2-c2ccccc2)cc1] | 3-{[({4-[(Biphenyl-2-yloxy)methyl]phenyl{carbamoyl) amino]methyl}-1-{[(pyrrolidin-1-ylcarbonyl)oxy] methyl}pyridinium |
| 17 | 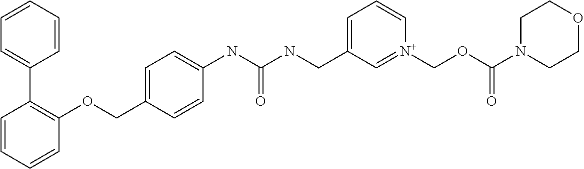 [SMILES: O=C(NCc1ccc[n+](COC(=O)N2CCOCC2)c1)Nc1ccc(COc2ccccc2-c2ccccc2)cc1] | 3-{[({4-[(Biphenyl-2-yloxy)methyl]phenyl}carbamoyl) amino]methyl}-1-{[(morpholin-4-ylcarbonyl)oxy] methyl}pyridinium |
| 18 | 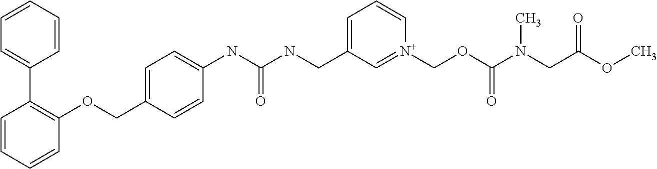 [SMILES: COC(=O)CN(C)C(=O)OC[n+]1cccc(CNC(=O)Nc2ccc(COc3ccccc3-c3ccccc3)cc2)c1] | Methyl N-{[(3-{[({4-[(biphenyl-2-yloxy)methyl]phenyl} carbamoyl)amino]methyl}pyridinium-1-yl)methoxy] carbonyl}-N-methylglycinate |
| 19 | 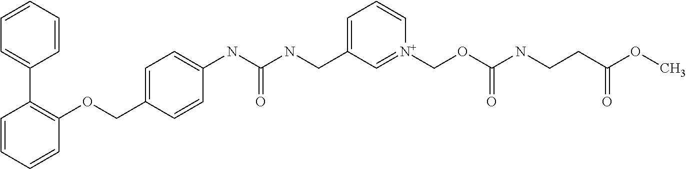 [SMILES: COC(=O)CCNC(=O)OC[n+]1cccc(CNC(=O)Nc2ccc(COc3ccccc3-c3ccccc3)cc2)c1] | Methyl N-{[(3-{[({4-[(biphenyl-2-yloxy)methyl]phenyl} carbamoyl)amino]methyl}pyridinium-1-yl)methoxy] carbonyl}-beta-alaninate |
| 20 | 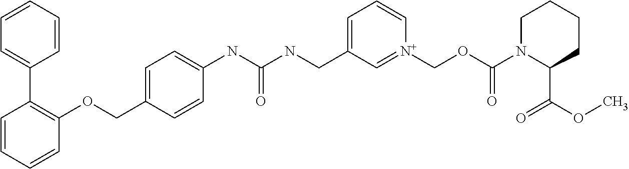 [SMILES: COC(=O)[C@@H]1CCCCN1C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[({[(2S)-2-(methoxycarbonyl)piperidin- 1-yl]carbonyl}oxy)methyl]pyridinium |
| 21 | 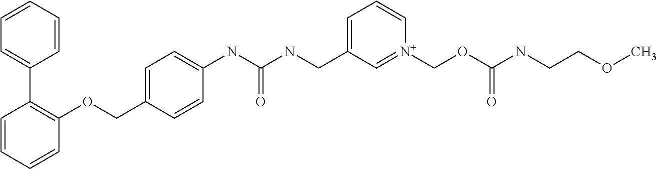 [SMILES: COCCNC(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-({[(2-methoxyethyl)carbamoyl]oxy} methyl)pyridinium |
| 22 | 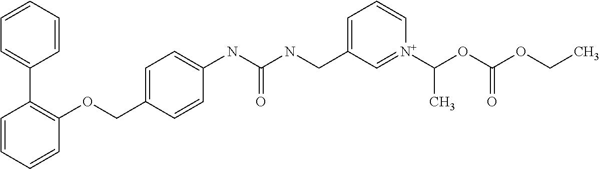 [SMILES: CCOC(=O)OC(C)[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{1-[(ethoxycarbonyl)oxy]ethyl} pyridinium |
| 23 | 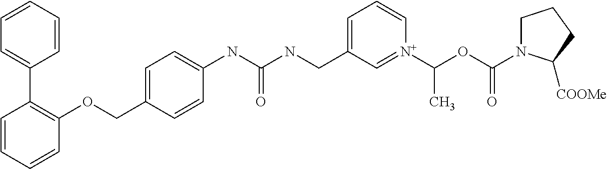 [SMILES: COC(=O)[C@@H]1CCCN1C(=O)OC(C)[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[1-({[(2S)-2-(methoxycarbonyl) pyrrolidin-1-yl]carbonyl}oxy)ethyl]pyridinium |
| 24 | 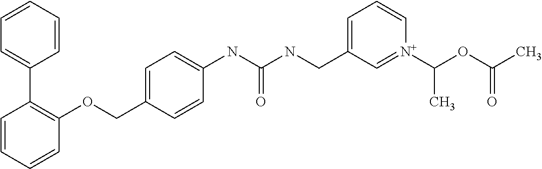 [SMILES: CC(=O)OC(C)[n+]1cccc(CNC(=O)Nc2ccc(COc3ccccc3-c3ccccc3)cc2)c1] | 1-[1-(Acetyloxy)ethyl]-3-{[({4-[(biphenyl-2-yloxy)methyl] phenyl}carbamoyl)amino]methyl}pyridinium |
| 25 | 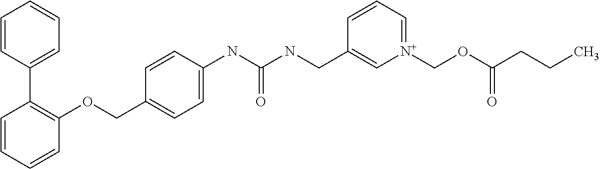 [SMILES: CCCC(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[(butanoyloxy)methyl]pyridinium |
| 26 | 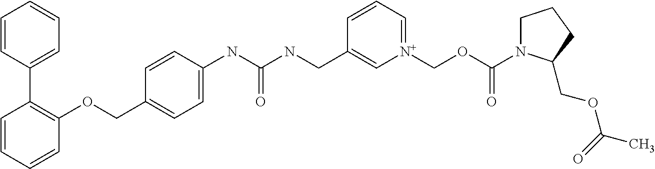 [SMILES: CC(=O)OC[C@@H]1CCCN1C(=O)OC[n+]1cccc(CNC(=O)Nc2ccc(COc3ccccc3-c3ccccc3)cc2)c1] | 1-{[({(2S)-2-[(Acetyloxy)methyl]pyrrolidin-1-yl} carbonyl)oxy]methyl}-3-{[({4-[(biphenyl-2-yloxy) methyl]phenyl}carbamoyl)amino]methyl}pyridinium |
| 27 | 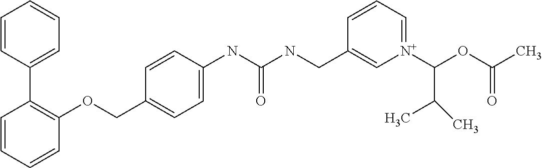 [SMILES: CC(=O)OC(C(C)C)[n+]1cccc(CNC(=O)Nc2ccc(COc3ccccc3-c3ccccc3)cc2)c1] | 1-[1-(Acetyloxy)-2-methylpropyl]-3-{[({4-[(biphenyl-2- yloxy)methyl]phenyl}carbamoyl)amino]methyl} pyridinium |
| 28 | 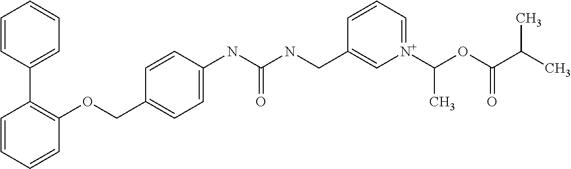 [SMILES: CC(C)C(=O)OC(C)[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{1-[(2-methylpropanoyl)oxy] ethyl}pyridinium |
| 29 | 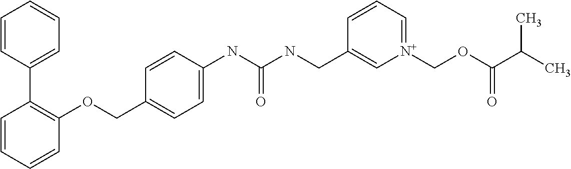 [SMILES: CC(C)C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-{[(2-methylpropanoyl)oxy] methyl}pyridinium |
| 30 | 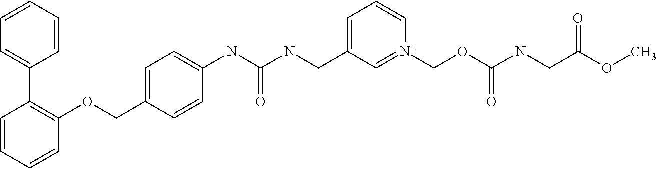 [SMILES: COC(=O)CNC(=O)OC[n+]1cccc(CNC(=O)Nc2ccc(COc3ccccc3-c3ccccc3)cc2)c1] | Methyl N-{[(3-{[({4-[(biphenyl-2-yloxy)methyl]phenyl} carbamoyl)amino]methyl}pyridinium-1-yl)methoxy] carbonyl}glycinate |
| 31 | 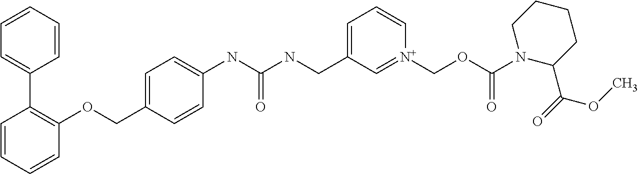 [SMILES: COC(=O)C1CCCCN1C(=O)OC[n+]1cccc(CNC(=O)Nc2ccc(COc3ccccc3-c3ccccc3)cc2)c1] | 3-{[({4-[(Biphenyl-2-yloxy)methyl]phenyl}carbamoyl) amino]methyl}-1-[({[2-(methoxycarbonyl)piperidin-1- yl]carbonyl}oxy)methyl]pyridinium |
| 32 | 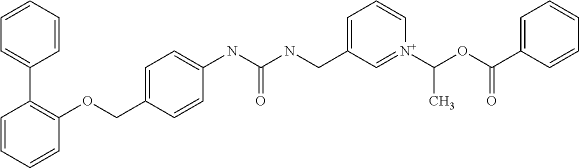 [SMILES: CC(OC(=O)c1ccccc1)[n+]1cccc(CNC(=O)Nc2ccc(COc3ccccc3-c3ccccc3)cc2)c1] | 1-[1-(Benzoyloxy)ethyl]-3-{[({4-[(biphenyl-2-yloxy) methyl]phenyl}carbamoyl)amino]methyl}pyridinium |
| 33 | 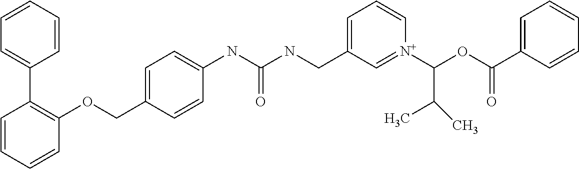 [SMILES: CC(C)C(OC(=O)c1ccccc1)[n+]1cccc(CNC(=O)Nc2ccc(COc3ccccc3-c3ccccc3)cc2)c1] | 1-[1-(Benzoyloxy)-2-methylpropyl]-3-{[((4-[(biphenyl-2- yloxy)methyl]phenyl}carbamoyl)amino]methyl} pyridinium |
| 34 | 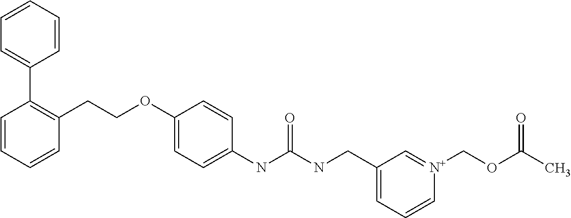 [SMILES: CC(=O)OC[n+]1cccc(CNC(=O)Nc2ccc(OCCc3ccccc3-c3ccccc3)cc2)c1] | 1-[(Acetyloxy)methyl]-3-{[({4-[2-(biphenyl-2-yl)ethoxy] phenyl}carbamoyl)amino]methyl}pyridinium |
| 35 | 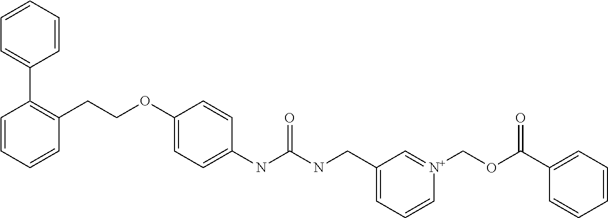 [SMILES: O=C(NCc1ccc[n+](COC(=O)c2ccccc2)c1)Nc1ccc(OCCc2ccccc2-c2ccccc2)cc1] | 1-[(Benzoyloxy)methyl]-3-{[({4-[2-(biphenyl-2- yl)ethoxy]phenyl}carbamoyl)amino]methyl} pyridinium |
| 36 | 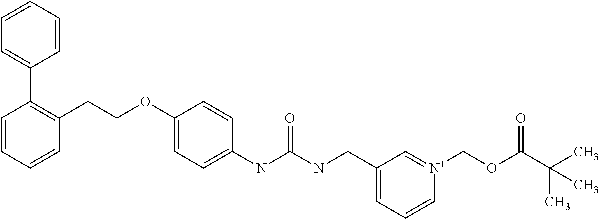 [SMILES: CC(C)(C)C(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{[(2,2-dimethylpropanoyl)oxy] methyl}pyridinium |
| 37 | 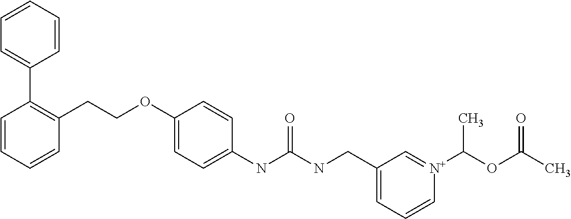 [SMILES: CC(=O)OC(C)[n+]1cccc(CNC(=O)Nc2ccc(OCCc3ccccc3-c3ccccc3)cc2)c1] | 1-[1-(Acetyloxy)ethyl]-3-{[({4-[2-(biphenyl-2-yl) ethoxy]phenyl}carbamoyl)amino]methyl}pyridinium |
| 38 | 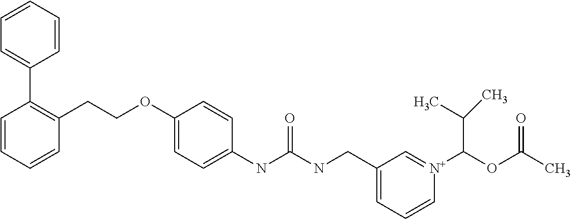 [SMILES: CC(=O)OC(C(C)C)[n+]1cccc(CNC(=O)Nc2ccc(OCCc3ccccc3-c3ccccc3)cc2)c1] | 1-[1-(Acetyloxy)-2-methylpropyl]-3-{[({4-[2- (biphenyl-2-yl)ethoxy]phenyl}carbamoyl)amino] methyl}pyridinium |
| 39 | 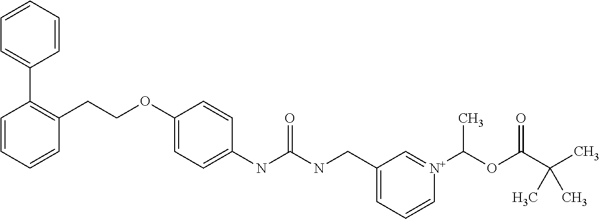 [SMILES: CC(OC(=O)C(C)(C)C)[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{1-[(2,2-dimethylpropanoyl) oxy]ethyl}pyridinium |
| 40 | 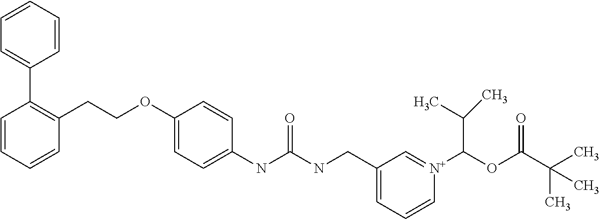 [SMILES: CC(C)C(OC(=O)C(C)(C)C)[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{1-[(2,2-dimethylpropanoyl)oxy]-2- methylpropyl}pyridinium |
| 41 | 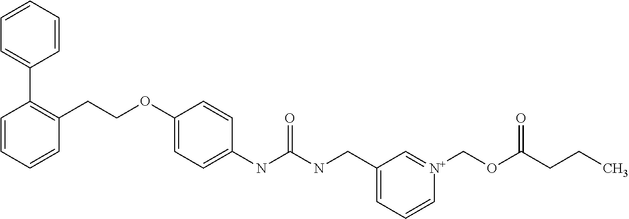 [SMILES: CCCC(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-[(butanoyloxy)methyl]pyridinium |
| 42 | 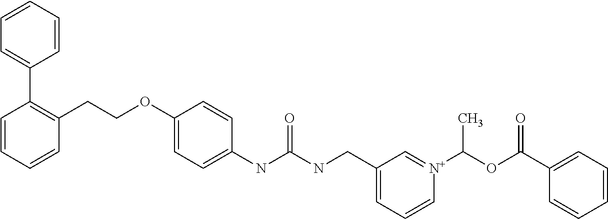 [SMILES: CC(OC(=O)c1ccccc1)[n+]1cccc(CNC(=O)Nc2ccc(OCCc3ccccc3-c3ccccc3)cc2)c1] | 1-[1-(Benzoyloxy)ethyl]-3-{[({4-[2-(biphenyl-2- yl)ethoxy]phenyl}carbamoyl)amino]methyl}pyridinium |
| 43 | 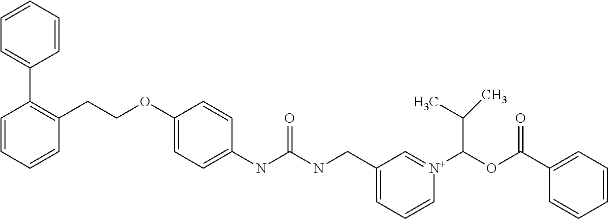 [SMILES: CC(C)C(OC(=O)c1ccccc1)[n+]1cccc(CNC(=O)Nc2ccc(OCCc3ccccc3-c3ccccc3)cc2)c1] | 1-[1-(Benzoyloxy)-2-methylpropyl]-3-{[({4-[2- (biphenyl-2-yl)ethoxy]phenyl}carbamoyl)amino]methyl} pyridinium |
| 44 | 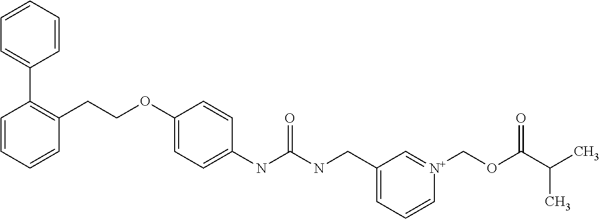 [SMILES: CC(C)C(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{[(2-methylpropanoyl)oxy] methyl}pyridinium |
| 45 | 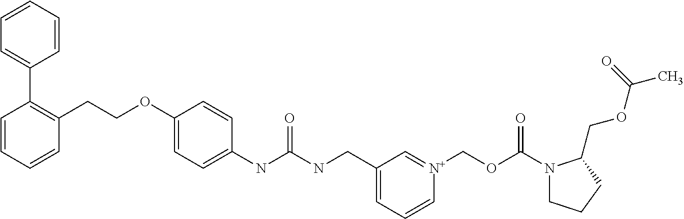 [SMILES: CC(=O)OC[C@@H]1CCCN1C(=O)OC[n+]1cccc(CNC(=O)Nc2ccc(OCCc3ccccc3-c3ccccc3)cc2)c1] | 1-{[({(2S)-2-[(Acetyloxy)methyl]pyrrolidin-1-yl} carbonyl)oxy]methyl}-3-{[({4-[2-(biphenyl-2-yl)ethoxy] phenyl}carbamoyl)amino]methyl}pyridinium |
| 46 | 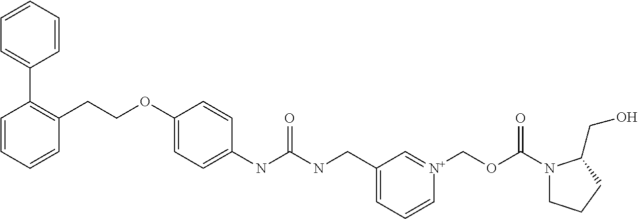 [SMILES: O=C(NCc1ccc[n+](COC(=O)N2CCC[C@H]2CO)c1)Nc1ccc(OCCc2ccccc2-c2ccccc2)cc1] | 3-{[({4-[2-(Biphenyl-2-yl)ethoxy]phenyl}carbamoyl) amino]methyl}-1-[({[(2S)-2-(hydroxymethyl)pyrrolidin- 1-yl]carbonyl}oxy)methyl]pyridinium |
| 47 | 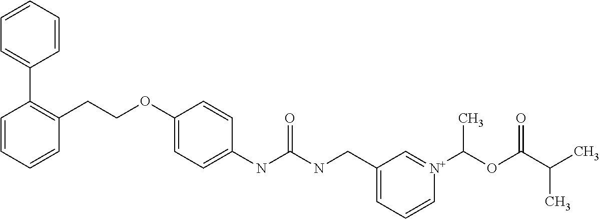 [SMILES: CC(C)C(=O)OC(C)[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{1-[(2-methylpropanoyl)oxy] ethyl}pyridinium |
| 48 | 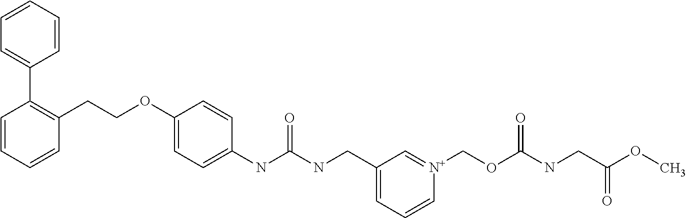 [SMILES: COC(=O)CNC(=O)OC[n+]1cccc(CNC(=O)Nc2ccc(OCCc3ccccc3-c3ccccc3)cc2)c1] | Methyl N-{[(3-{[({4-[2-(biphenyl-2-yl)ethoxy]phenyl} carbamoyl)amino]methyl}pyridinium-1-yl)methoxy] carbonyl}glycinate |
| 49 | 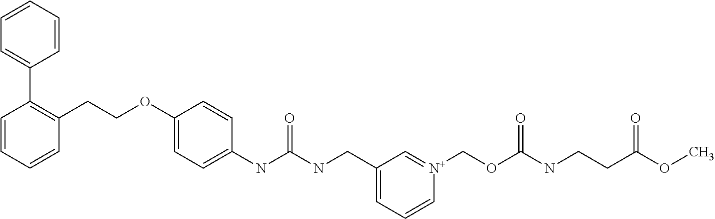 [SMILES: COC(=O)CCNC(=O)OC[n+]1cccc(CNC(=O)Nc2ccc(OCCc3ccccc3-c3ccccc3)cc2)c1] | Methyl N-{[(3-{[({4-[2-(biphenyl-2-yl)ethoxy]phenyl} carbamoyl)amino]methyl}pyridinium-1-yl)methoxy] carbonyl}-beta-alaninate |
| 50 | 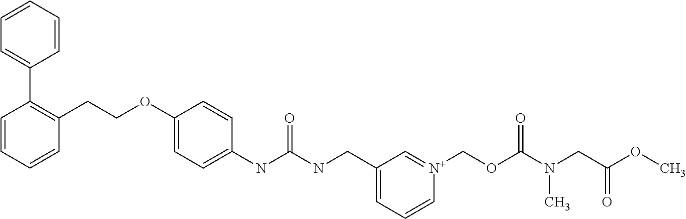 [SMILES: COC(=O)CN(C)C(=O)OC[n+]1cccc(CNC(=O)Nc2ccc(OCCc3ccccc3-c3ccccc3)cc2)c1] | Methyl N-{[(3-{[({4-[2-(biphenyl-2-yl)ethoxy]phenyl} carbamoyl)amino]methyl}pyridinium-1-yl)methoxy] carbonyl}-N-methylglycinate |
| 51 | 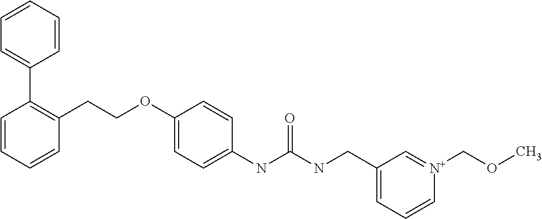 [SMILES: COC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-(methoxymethyl)pyridinium |
| 52 | 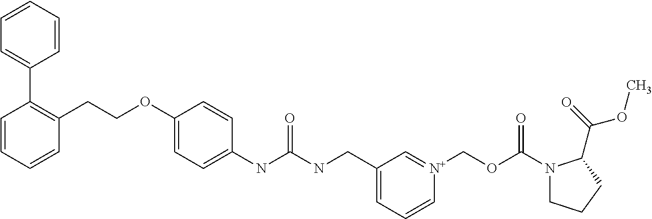 [SMILES: COC(=O)[C@@H]1CCCN1C(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-[({[(2S)-2-(methoxycarbonyl) pyrrolidin-1-yl]carbonyl}oxy)methyl]pyridinium |
| 53 | 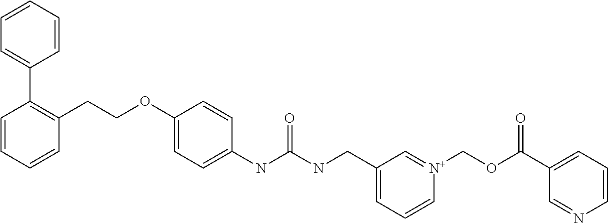 [SMILES: O=C(NCc1ccc[n+](COC(=O)c2cccnc2)c1)Nc1ccc(OCCc2ccccc2-c2ccccc2)cc1] | 3-{[({4-[2-(Biphenyl-2-yl)ethoxy]phenyl)carbamoyl) amino]methyl}-1-{[(pyridin-3-ylcarbonyl)oxy] methyl}pyridinium |
| 54 | 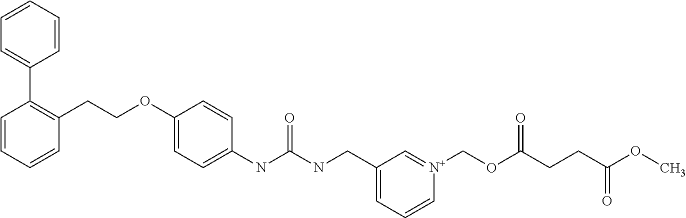 [SMILES: COC(=O)CCC(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{[(4-methoxy-4-oxobutanoyl)oxy] methyl}pyridinium |
| 55 | 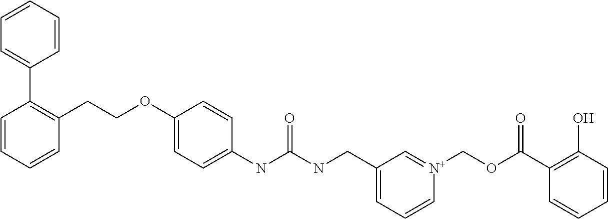 [SMILES: O=C(NCc1ccc[n+](COC(=O)c2ccccc2O)c1)Nc1ccc(OCCc2ccccc2-c2ccccc2)cc1] | 3-{[({4-[2-(Biphenyl-2-yl)ethoxy]phenyl}carbamoyl) amino]methyl}-1-{[(2-hydroxybenzoyl)oxy] methyl}pyridinium |
| 56 | 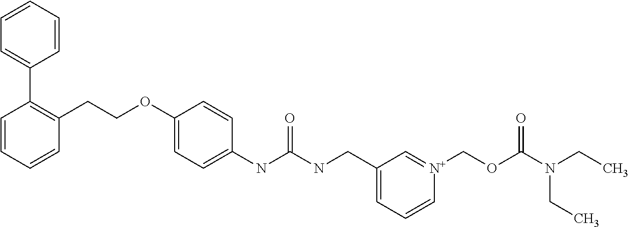 [SMILES: CCN(CC)C(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-{[(diethylcarbamoyl)oxy] methyl}pyridinium |
| 57 | 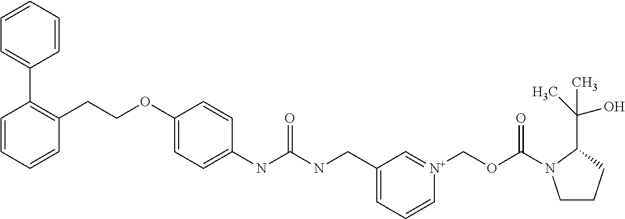 [SMILES: CC(C)(O)[C@@H]1CCCN1C(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-[({[(2S)-2-(2-hydroxypropan-2-yl) pyrrolidin-1-yl]carbonyl}oxy)methyl]pyridinium |
| 58 | 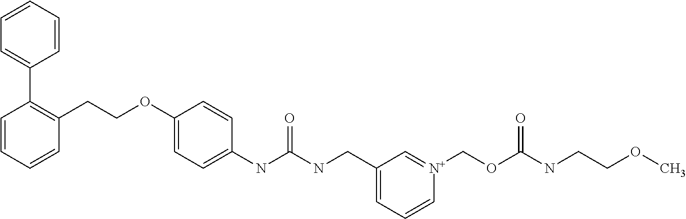 [SMILES: COCCNC(=O)OC[n+]1cccc(CNC(=O)Nc2ccc(OCCc3ccccc3-c3ccccc3)cc2)c1] | 3-{[({4-[2-(Biphenyl-2-yl)ethoxy]phenyl}carbamoyl) amino]methyl}-1-({[(2-methoxyethyl)carbamoyl]oxy} methyl)pyridinium |
| 59 | 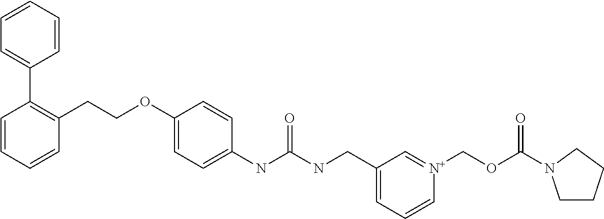 [SMILES: O=C(NCc1ccc[n+](COC(=O)N2CCCC2)c1)Nc1ccc(OCCc2ccccc2-c2ccccc2)cc1] | 3-{[({4-[2-(Biphenyl-2-yl)ethoxy]phenyl}carbamoyl) amino]methyl}-1-{[(pyrrolidin-1-ylcarbonyl)oxy] methyl}pyridinium |
| 60 | 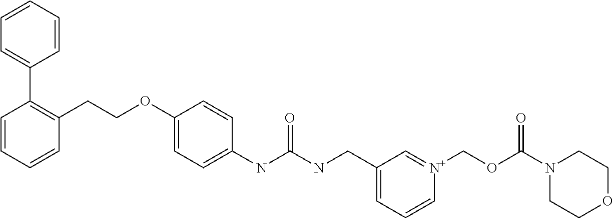 [SMILES: O=C(NCc1ccc[n+](COC(=O)N2CCOCC2)c1)Nc1ccc(OCCc2ccccc2-c2ccccc2)cc1] | 3-{[({4-[2-(Biphenyl-2-yl)ethoxy]phenyl}carbamoyl) amino]methyl}-1-{[(morpholin-4-ylcarbonyl)oxy] methyl}pyridinium |

TABLE 1B

| Ex. | Synthetic | | HRMS | HRMS |
|---|---|---|---|---|
| No. | Procedure | NMR Data | Calculated | Found |
| 1 | E, A | 9.82 (s, 1H), 9.14 (s, 1H), 9.11 (d, | 723.26999 | 723.2610 |
| | | 1H), 8.83 (s, 1H), s, 1H), 8.21 (dd, | | |
| | | 1H), 7.95 (s, 1H), 7.62 (td, 1H), 7.54 | | |
| | | (td, 1H), 7.42-7.36 (m, 3H), 7.28 (dd, | | |
| | | 1H), 7.26-7.21 (m, 3H), 6.88-6.80 (m, | | |
| | | 3H), 6.57 (bs, 2H), 6.45 (s, 2H), 4.49 | | |
| | | (d, 2H), 4.27-4.23 (m, 2H), 3.64-3.62 | | |
| | | (m, 2H), 3.54-3.46 (m, 12H) | | |
| 2 | A | 9.13 (s, 1H), 9.09 (d, 1H), 9.06 (s, | 482.2163 | 482.2203 |
| | | 1H), 8.63 (d, 1H), 8.20 (dd, 1H), 7.51 | | |
| | | (d, 2H), 7.41-7.27 (m, 8H), 7.24 (d, | | |
| | | 2H), 7.18 (d, 1H), 7.04(d, 1H), 7.00 | | |
| | | (t, 1H), 6.42 (s, 2H), 5.02 (s, 2H), | | |
| | | 4.51 (d, 2H), 2.15 (s, 3H) | | |
| 3 | A | 9.13-9.07 (m, 2H), 8.65 (d, 1H), 8.21 | 524.2549 | 524.269 |
| | | (dd, 1H), 7.51 (dd, 2H), 7.41-7.27 (m, | | |
| | | 8H), 7.23 (d, 2H), 7.18 (d, 1H), 7.06- | | |
| | | 7.00 (m, 2H), 6.44 (s, 2H), 5.02 (s, | | |
| | | 2H), 4.52 (d, 2H), 1.14 (s, 9H) | | |
| 4 | E, A | 9.16 (s, 1H), 9.12 (d, lH), 9.03 (s, | 674.3078 | 674.3051 |
| | | 1H), 8.66 (d, 1H), 8.21 (dd, 1H), 7.51 | | |
| | | (d, 2H), 7.41-7.27 (m, 8H), 7.24 (d, | | |
| | | 2H), 7.18 (d, 1H), 7.03 (t, 1H), 6.98 | | |
| | | (t, 1H), 6.46 (s, 2H), 5.02 (s, 2H), | | |
| | | 4.52 (d, 2H), 4.28-4.23 (m, 2H), 3.63- | | |
| | | 3.59 (m, 2H), 3.55-3.45 (m, 12H), | | |
| | | 3.22 (s, 3H) | | |
| 5 | E, A | 9.15 (s, 1H), 9.11 (d, 1H), 9.09 (s, | 498.2029 | 498.2097 |
| | | 1H), 8.65 (d, 1H), 8.21 (dd, 1H), 7.51 | | |
| | | (d, 2H), 7.41-7.27 (m, 8H), 7.24 (d, | | |
| | | 2H), 7.18 (d, 1H), 7.03 (td, 2H), 6.46 | | |
| | | (s, 2H), 5.02 (s, 2H), 4.52 (d, 2H), | | |
| | | 3.77 (s, 3H) | | |
| 6 | A | 9.09 (s, 1H), 9.00 (s, 1H), 8.96 (d, | 522.2029 | 522.2091 |
| | | 1H), 8.57 (d, 1H), 8.18 (dd, 1H), 7.51 | | |
| | | (d, 2H), 7.41-7.27 (m, 8H), 7.24 (d, | | |
| | | 2H), 7.18 (d, 1H), 7.03 (td, 2H), 5.88 | | |
| | | (s, 2H), 5.02 (s, 2H), 4.51 (d, 2H), | | |
| | | 2.30 (s, 3H) | | |
| 7 | D, A | 9.13-9.00 (m, 3H), 8.63 (d, 1H), 8.24- | 595.2557 | 595.2532 |
| | | 8.17 (m, 1H), 7.51 0d, 2H), 7.41-7.27 | | |
| | | (m, 8H), 7.24 (d, 2H), 7.18 (d, 1H), | | |
| | | 7.03 (t, 2H), 6.50-6.34 (m, 2H), 5.02 | | |
| | | (s, 2H), 4.52 (d, 2H), 3.61 (d, 3H), | | |
| | | 3.52-3.34 (m, 2H), 1.97-1.69 (m, 4H) | | |
| 8 | D, A | 9.08 (s, 1H), 9.02 (d, 1H), 9.00 (s, | ? | 637.3021 |
| | | 1H), 8.63 (d, 1H), 8.21 (dd, 1H), 7.98 | | |
| | | (d, 1H), 7.51 (d, 2H), 7.41-7.27 (m, | | |
| | | 8H), 7.24 (d, 2H), 7.18 (d, 1H), 7.03 | | |
| | | (t, 1H), 6.96 (t, 1H), 6.38 (q, 2H), | | |
| | | 5.02 (s, 2H), 4.51 (d, 2H), 4.16-3.95 | | |
| | | (m, 2H), 3.86 (qd, 2H), 1.80-1.57 (m, | | |
| | | 2H), 1.57-1.44 (m, 4H), 1.1.37-1.16 | | |
| | | (m, 2H), 0.96 (t, 3H) | | |
| 9 | D, A | 9.08 (s, 1H), 9.02 (d, 1H), 9.00 (s, | ? | 637.3026 |
| | | 1H), 8.63 (d, 1H), 8.21 (dd, 1H), 7.98 | | |
| | | (d, 1H), 7.51 (d, 2H), 7.41-7.27 (m, | | |
| | | 8H), 7.24 (d, 2H), 7.18 (d, 1H), 7.03 | | |
| | | (t, 1H), 6.96 (t, 1H), 6.36 (q, 2H), | | |
| | | 5.02 (s, 2H), 4.51 (d, 2H), 4.16-3.99 | | |
| | | (m, 2H), 3.88 (qd, 2H), 1.88-1.68 (m, | | |
| | | 2H), 1.68-1.56 (m, 4H), 1.42-1.16 (m, | | |
| | | 2H), 0.96 (t, 3H) | | |
| 10 | D, A | 9.14-8.98 (m, 3H), 8.63 (d, 1H), 8.23- | 771.35997 | 771.3591 |
| | | 8.17 (m, 1H), 7.51 (d, 2H), 7.41-7.27 | | |
| | | (m, 8H), 7.24 (d, 2H), 7.18 (d, 1H), | | |
| | | 7.03 (t, 1H), 6.99 (bs, 1H), 6.50-6.34 | | |
| | | (m, 2H), 5.02 (s, 2H), 4.52 (d, 2H), | | |
| | | 3.61 (d, 2H), 3.56 (q, 1H), 3.52-3.45 | | |
| | | (m, 8H), 3.43-3.36 (m, 15H, includes | | |
| | | H2O), 3.21 (d, 2H), 3.17 (s, 3H), | | |
| | | 1.98-1.77 (m, 4H) | | |
| 11 | C, A | 9.25-9.19 (m, 2H), 9.04 (s, 1H), 8.61 | 538.270033 | 538.2704 |
| | | (d, 1H), 8.21 (t, 1H), 7.51 (d, 2H), | | |
| | | 7.41-7.27 (m, 7H), 7.24 (d, 2H), 7.18 | | |
| | | (d, 1H), 7.08-6.97 (m, 3H), 5.02 (s, | | |
| | | 2H), 4.52 (d, 2H), 1.88 (d, 3H), 1.15 | | |
| | | (s, 9H) | | |
| 12 | C, A | 9.18 (d, 1H), 9.13 (S, 1H), 8.95 (s, | 566.301333 | 566.3024 |
| | | 1H), 8.65 (d, 1H), 8.24 (dd, 1H), 7.51 | | |
| | | (d, 2H), 7.42-7.27 9m, 7H), 7.23 (d, | | |
| | | 2H), 7.18 (d, 1H), 7.03 (t, 1H), 6.92 | | |
| | | (t, 1H), 6.78 (d, 1H), 5.03 (s, 2H), | | |
| | | 4.52 (d, 2H), 1.18 (s, 9H), 1.06 (d, | | |
| | | 3H), 0.77 (d, 3H) | | |
| 13 | A | 9.25 (s, 1H), 9.23 (d, 1H), 9.00 (d, | 544.22363 | 544.2211 |
| | | 1H), 8.65 (d, 1H), 8.23 (dd, 1H), 8.02 | | |
| | | (d, 2H), 7.71 (t, 1H), 7.58-7.49 (m, | | |
| | | 4H), 7.41-7.27 (m, 8H), 7.23 (d, 2H), | | |
| | | 7.18 (d, 1H), 7.03 (t, 1H), 6.95 (t, | | |
| | | 1H), 6.69)s, 2H), 5.02 (s, 2H), 4.53 | | |
| | | (d, 2H) | | |
| 14 | D, A | 9.09 (s, 1H), 9.06 (d, 1H), 9.00 (s, | 497.218332 | 497.2195 |
| | | 1H), 8.62 (d, 1H), 8.19 (dd, 1H), 7.78 | | |
| | | (q, 1H), 7.51 (d, 2H), 7.41-7.27 (m, | | |
| | | 8H0, 7.24 (d, 2H), 7.18 (d, 1H), 7.03 | | |
| | | (t, 1H), 6.96 (t, 1H), 6.36 (s, 2H), 5.03 | | |
| | | (s, 2H), 4.51 (d, 2H), 2.58 (d, 3H) | | |
| 15 | D, A | 9.12-9.07 (m, 2H), 9.02 (bs, 1H), 8.61 | 511.233982 | 511.235 |
| | | (d, 1H), 8.18 (dd, 1H), 7.51 (d, 2H), | | |
| | | 7.42-7.27 (m, 7H), 7.24 (d, 2H), 7.18 | | |
| | | (d, 1H), 7.03 (t, 1H), 6.97 (bs, 1H), | | |
| | | 6.38 (s, 2H), 5.02 (s, 2H), 4.51 (d, | | |
| | | 2H), 2.90 (s, 3H), 2.80 (s, 3H) | | |
| 16 | D, A | 9.12-9.04 (m, 3H), 8.61 (d, 1H), 8.19 | 537.249632 | 537.2499 |
| | | (dd, 1H), 7.51 (d, 2H), 7.42-7.27 (m, | | |
| | | 7H), 7.24 (d, 2H), 7.18 (d, 1H), 7.06- | | |
| | | 6.98 (m, 2H), 6.40 (s, 2H), 5.02 (s, | | |
| | | 2H), 4.52 (d, 2H), 3.23 (t, 2H), 3.09 | | |
| | | (t, 2H), 1.80-1.70 (m, 4H) | | |
| 17 | D, A | 9.13-9.08 (m, 2H), 8.97 (s, 1H), 8.62 | 553.244547 | 553.2542 |
| | | (d, 1H), 8.19 (dd, 1H), 7.51 (d, 2H), | | |
| | | 7.41-7.27 (m, 7H), 7.24 (d, 2H), 7.18 | | |
| | | (d, 1H), 7.03 (t, 1H), 6.92 (t, 1H), | | |
| | | 6.41 (s, 2H), 5.02 (s, 2H), 4.51 (d, | | |
| | | 2H), 3.57-3.52 (m, 4H), 3.47-3.41 (m, | | |
| | | 2H), 3.34-3.28 (m, 2H) | | |
| 18 | D, A | 9.16-9.01 (m, 3H), 8.62 (d, 1H), 8.20 | 569.239461 | 569.2412 |
| | | (t, 1H), 7.51 (d, 2H), 7.41-7.27 (m, | | |
| | | 8H), 7.24 (d, 2H), 7.18 (d, 1H), 7.03, | | |
| | | (t, 1H), 6.98 (t, 1H), 6.42 (d, 2H), | | |
| | | 5.02 (s, 2H), 4.51 (d, 2H), 4.11 (d, | | |
| | | 2H), 3.64 (d, 3H), 2.90 (d, 3H) | | |
| 19 | D, A | 9.12-9.03 (m, 3H), 8.63 (d, 1H), 8.20 | 569.239461 | 569.2416 |
| | | (dd, 1H), 8.01 (t, 1H), 7.51 (d, 2H), | | |
| | | 7.41-7.27 (m, 8H), 7.24 (d, 2H), 7.18 | | |
| | | (d, 1H), 7.06-6.98 (m, 2H), 6.37 (s, | | |
| | | 2H), 5.02 (s, 2H), 4.52 (d, 2H), 3.82- | | |
| | | 3.73 (m, 1H), 3.55 (s, 3H), 3.26-3.18 | | |
| | | (m, 2H), 2.50-2.43 (m, 2H) | | |
| 20 | D, A | n/a | 609.270761 | 609.2709 |
| 21 | D, A | 9.12-9.03 (m, 3H), 8.62 (d, 1H), 8.20 | 541.244547 | |
| | | (dd, 1H), 7.99 (t, 1H), 7.51 (d, 2H), | | |
| | | 7.44-7.27 (m, 8H), 7.24 (d, 2H), 7.18 | | |
| | | (d, 1H), 7.07-7.00 (m, 2H), 6.38 (s, | | |
| | | 2H), 5.02 (s, 2H), 4.51 (d, 2H), 3.32- | | |
| | | 3.29 (m, 2H), 3.20-3.15 (m, 5H) | | |
| 22 | A | 9.27 (s, 1H), 9.21 (d, 1H), 9.09 (s, | 526.233648 | 526.2479 |
| | | 1H), 8.61 (d, 1H), 8.21 (dd, 1H), 7.51 | | |
| | | (d, 2H), 7.41-7.27 (m, 8H), 7.24 (d, | | |
| | | 2H), 7.18 (d, 1H), 7.06-6.97 (m, 3H), | | |
| | | 5.02 (s, 2H), 4.53 (d, 2H), 4.22-4.07 | | |
| | | (m, 2H), 3.18 (d, 1H), 1.90 (d, 3H), | | |
| | | 1.19 (t, 3H) | | |
| 23 | D, A | 9.23-9.14 (m, 2H), 8.98 (s, 1H), 8.58 | 609.270761 | 609.2799 |
| | | (d, 1H), 8.19 (dd, 1H), 7.51 (d, 2H), | | |
| | | 7.41-7.27 (m, 8H), 7.24 (d, 2H), 7.18 | | |
| | | (d, 1H), 7.06-6.90 (m, 3H), 5.03 (s, | | |
| | | 2H), 4.52 (d, 2H), 4.12 (q, 1H), 3.73 | | |
| | | (d, 3H), 3.66-3.42 (m, 2H), 1.92-1.71 | | |
| | | (m, 7H) | | |
| 24 | C, A | 9.22 (s, 1H), 9.19 (d, 1H), 9.01 (s, | 496.223083 | 496.2288 |
| | | 1H), 8.58 (d, 1H), 8.19 (dd, 1H), 7.51 | | |
| | | (d, 2H), 7.41-7.72 (m, 8H), 7.24 (d, | | |
| | | 2H), 7.18 (d, 1H), 7.08-7.00 (m, 2H), | | |
| | | 6.96 (t, 1H), 5.02 (s, 2H), 4.52 (d, | | |
| | | 2H), 2.14 (s, 3H), 1.86 (d, 3H) | | |
| 25 | A | 9.13 (s, 1H), 9.10 (d, 1H), 9.08 (s, | 510.238733 | 510.2363 |
| | | 1H), 8.63 (d, 1H), 8.20 (dd, 1H), 7.51 | | |
| | | (d, 2H), 7.41-7.27 (m, 8H, 7.24 (d, | | |
| | | 2H), 7.18 (d, 1H), 7.06-7.00 (m, 2H), | | |
| | | 6.44 (s, 2H), 5.02 (s, 2H), 4.51 (d, | | |
| | | 2H), 2.42 (t, 2H), 1.53 (q, 2H), 0.85 | | |
| | | (t, 3H) | | |
| 26 | F, A | 9.14-9.06 (m, 3H), 8.62 (d, 1H), 8.19 | 609.270761 | 609.2698 |
| | | (dd, 1H), 7.51 (d, 2H), 7.41-7.27 (m, | | |
| | | 8H), 7.24 (d, 2H), 7.18 (d, 1H), 7.06- | | |
| | | 7.00 (m, 2H), 6.46-6.35 (m, 2H), 5.03 | | |
| | | (s, 1H), 4.52 (d, 2H), 4.18-4.02 (m, | | |
| | | 1H), 4.00-3.92 (m, 2H), 3.42-3.23 (m, | | |
| | | 2H), 1.96 (d, 3H), 1.90-1.69 (m, 4H) | | |
| 27 | C, A | 9.19-9.13 (m, 2H), 9.01 (s, 1H), 8.62 | 524.254383 | 524.2534 |
| | | (d, 1H), 8.22 (dd, 1H), 7.51 (d, 2H), | | |
| | | 7.41-7.27 (m, 8H), 7.24 (d, 2H), 7.18 | | |
| | | (d, 1H), 7.04 (t, 1H), 6.97 (t, 1H), | | |
| | | 6.78 (d, 1H), 5.02 (s, 2H), 4.52 (d, | | |
| | | 2H), 2.46-2.46 (m, 1H), 1.05 (d, 3H), | | |
| | | 0.74 (d, 3H) | | |
| 28 | C, A | 9.23-9.19 (m, 2H), 9.04 (s, 1H), 8.60 | 524.254383 | 524.2549 |
| | | (d, 1H), 8.20 (dd, 1H), 7.51 (d, 2H), | | |
| | | 7.41-7.27 (m, 8H), 7.24 (d, 2H), 7.18 | | |
| | | (d, 1H), 7.09-6.98 (m, 3H), 5.02 (s, | | |
| | | 2H), 4.52 (d, 2H), 2.71-2.62 (m, 1H), | | |
| | | 1.87 (d, 3H), 1.10 (d, 3H), 1.05 (d, | | |
| | | 3H) | | |
| 29 | B, A | 9.13 (m, 3H), 8.64 (d, 1H), 8.20 (dd, | 510.238733 | 510.237 |
| | | 1H), 7.51 (d, 2H), 7.41-7.27 (m, 8H), | | |
| | | 7.24 (d, 2H), 7.18 (d, 1H), 7.03 (t, | | |
| | | 1H), 6.98 (t, 1H), 6.44 (s, 2H), 5.02 | | |
| | | (d, 2H), 4.51 (d, 2H), 2.71-2.62 (m, | | |
| | | 1H), 1.09 (d, 6H) | | |
| 30 | D, A | 9.12 (s, 1H), 9.10 (s, 1H), 9.06 (d, | 555.223811 | 555.2244 |
| | | 1H), 8.64 (d, 1H), 8.39 (t, 1H), 8.21 | | |
| | | (dd, 1H), 7.51 (d, 2H), 7.41-7.27 (m, | | |
| | | 8H), 7.24 (d, 2H), 7.18 (d, 1H), 7.07- | | |
| | | 7.00 (m, 2H), 6.42 (s, 2H), 5.02 (s, | | |
| | | 2H), 4.52 (d, 2H), 3.80 (d, 2H), 3.62 | | |
| | | (s, 3H) | | |
| 31 | D, A | 9.16-9.02 (m, 3H), 8.63 (t, 1H), 8.20 | 609.270761 | 609.2699 |
| | | (td, 1H), 7.51 (d, 2H), 7.41-7.27 (m, | | |
| | | 8H), 7.24 (d, 2H), 7.18 (d, 1H), 7.06- | | |
| | | 6.98 (m, 2H), 6.50-6.37 (m, 2H), 5.02 | | |
| | | (s, 2H), 4.52 (d, 2H), 3.83-3.71 (m, | | |
| | | 3H), 3.63 (s, 3H), 1.68-1.63 (m, 2H), | | |
| | | 1.38-1.06 (m, 4H) | | |
| 32 | C, A | 9.38 (s, 1H), 9.34 (d, 1H), 9.04 (s, | 558.238733 | 558.2377 |
| | | 1H), 8.60 (d, 1H), 8.21 (dd, 1H), 8.07 | | |
| | | (d, 2H), 7.71 (t, 1H), 7.58-7.49 (m, | | |
| | | 4H), 7.41-7.26 (m, 9H), 7.22 (d, 2H), | | |
| | | 7.18 (d, 1H), 7.03 (t, 1H), 7.00 (t, | | |
| | | 1H), 5.02 (s, 2H), 4.53 (d, 2H), 2.02 | | |
| | | (d, 3H) | | |
| 33 | C, A | 9.35-9.30 (m, 2H), 9.10 (s, 1H), 8.64 | 586.270033 | 586.2692 |
| | | (d, 1H), 8.24 (dd, 1H), 8.11 (d, 2H), | | |
| | | 7.73 (t, 1H), 7.60-7.49 (m, 4H), 7.41- | | |
| | | 7.26 (m, 8H), 7.22 (d, 2H), 7.18 (d, | | |
| | | 1H), 7.09-7.01 (m, 3H), 5.02 (s, 2H), | | |
| | | 4.45 (d, 2H), 2.70-2.60 (m, 1H), 1.14 | | |
| | | (d, 3H), 0.84 (d, 3H) | | |
| 34 | A | 9.12 (s, 1H), 9.09 (d, 1H), 8.78 (s, | 496.223083 | 496.2233 |
| | | 1H), 8.62 (d, 1H), 8.20 (dd, 1H), | | |
| | | 7.48-7.27 (m, 8H), 7.25-7.18 (m, 3H), | | |
| | | 6.87 (bs, 1H), 6.64 (d, 2H), 6.42 (s, | | |
| | | 2H), 4.49 (d, 2H), 3.95 (t, 2H), 2.96 | | |
| | | (t, 2H), 2.15 (s, 3H) | | |
| 35 | A | 9.24 (s, 1H), 9.22 (d, 1H), 8.75 (s, | 558.238733 | 558.2385 |
| | | 1H), 8.64 (d, 1H), 8.22 (dd, 1H), 8.02 | | |
| | | (d, 2H), 7.73 (t, 1H), 7.56 (t, 2H), | | |
| | | 7.48-7.28 (m, 8H), 7.24-7.18 (m, 3H), | | |
| | | 6.86 (bs, 1H), 6.69 (s, 2H), 6.63 (d, | | |
| | | 2H), 4.50 (d, 2H), 3.95 (t, 2H), 2.96 | | |
| | | (t, 2H) | | |
| 36 | A | 9.12-9.08 (m, 2H), 8.71 (s, 1H), 8.63 | 538.270033 | 538.271 |
| | | (d, 1H), 8.21 (dd, 1H), 7.48-7.27 (m, | | |
| | | 8H), 7.24-7.18 (m, 3H), 6.81 (t, 1H), | | |
| | | 6.64 (d, 2H), 6.44 (s, 2H), 4.49 (d, | | |
| | | 2H), 3.95 (t, 2H), 2.96 (t, 2H), 1.15 (s, | | |
| | | 9H) | | |
| 37 | C, A | 9.21 (s, 1H), 9.18 (d, 1H), 8.74 (bs, | 510.238733 | 510.2401 |
| | | 1H), 8.57 (d, 1H), 8.19 (dd, 1H), | | |
| | | 7.49-7.28 (m, 8H), 7.25-7.18 (m, 3H), | | |
| | | 7.05 (q, 1H), 6.84 (bs, 1H), 6.64 (d, | | |
| | | 2H), 4.50 (d, 2H), 3.95 (t, 2H), 2.96 | | |
| | | (t, 2H), 2.14 (s, 3H), 1.85 (d, 3H) | | |
| 38 | C, A | 9.17-9.13 (m, 2H), 8.70 (d, 1H), 8.61 | 538.270033 | 538.2722 |
| | | (d, 1H), 8.21 (dd, 1H), 7.48-7.28 (m, | | |
| | | 8H), 7.23-7.18 (m, 3H), 6.82-6.76 (m, | | |
| | | 2H), 6.64 (d, 2H), 4.50 (d, 2H), 3.95 | | |
| | | (t, 2H), 2.96 (t, 2H), 2.46-2.41 (m, | | |
| | | 1H), 2.20 (s, 3H), 1.05 (d, 3H), 0.75 | | |
| | | (d, 3H) | | |
| 39 | C, A | 9.23-9.19 (m, 2H), 8.73 (s, 1H), 8.60 | 552.285683 | 552.2898 |
| | | (d, 1H), 8.21 (d, 1H), 8.21 (dd, 1H), | | |
| | | 7.49-7.27 (m, 8H), 7.24-7.18 (m, 3H), | | |
| | | 7.05 (q, 1H), 6.83 (t, 1H), 6.64 (d, | | |
| | | 2H), 4.50 (d, 2H), 3.95 (t, 2H), 2.96 | | |
| | | (t, 2H), 1.87 (d, 3H), 1.15 (s, 9H) | | |
| 40 | C, A | 9.17 (d, 1H), 9.12 (s, 1H), 8.72 (s, | 580.316983 | 580.3188 |
| | | 1H), 8.64 (d, 1H), 8.24 (dd, 1H), | | |
| | | 7.48-7.27 (m, 8H), 7.23-7.17 (m, 3H), | | |
| | | 6.83 (t, 1H), 6.78 (d, 1H), 6.63 (d, | | |
| | | 2H), 4.50 (d, 2H), 3.95 (t, 2H), 2.96 | | |
| | | (t, 2H), 1.18 (s, 9H), 1.06 (d, 3H), | | |
| | | 0.77 (d, 3H) | | |
| 41 | A | 9.11 (s, 1H), 9.09 (d, 1H), 8.71 (s, | 524.254383 | 524.2570 |
| | | 1H), 8.62 (d, 1H), 8.20 (dd, 1H), | | |
| | | 7.49-7.27 (m, 8H), 7.25-7.18 (m, 3H), | | |
| | | 6.81 (t, 1H), 6.64 (d, 2H), 6.44 (s, | | |
| | | 2H), 4.49 (d, 2H), 3.95 (t, 2H), 2.96 | | |
| | | (t, 2H), 2.42 (t, 2H), 1.54 (q, 2H), | | |
| | | 0.85 (t, 3H) | | |
| 42 | C, A | 9.37 (s, 1H), 9.33 (d, 1H), 8.70 (d, | 572.254383 | 572.2567 |
| | | 1H), 8.59 (d, 1H), 8.21 (dd, 1H), 8.07 | | |
| | | (d, 2H), 7.74 (t, 1H), 7.57 (t, 2H), | | |
| | | 7.48-7.28 (m, 9H), 7.22-7.16 (m, 3H), | | |
| | | 6.79 (t, 1H), 6.62 (d, 2H), 4.51 (d, | | |
| | | 2H), 3.95 (t, 2H), 2.96 (t, 2H), 2.01 | | |
| | | (d, 3H) | | |
| 43 | C, A | 9.33-9.29 (m, 2H), 8.78 (bs, 1H), 8.63 | 600.285683 | 600.2873 |
| | | (d, 1H), 8.49 (d, 1H), 8.24 (dd, 1H), | | |
| | | 8.11 (d, 2H), 7.76 (t, 1H), 7.59 (t, | | |
| | | 2H), 7.48-7.26 (m, 8H), 7.23-7.15 (m, | | |
| | | 3H), 7.07 (d, 1H), 6.87 (bs, 1H), 6.62 | | |
| | | (d, 2H), 4.51 (d, 2H), 3.95 (t, 2H), | | |
| | | 2.96 (t, 2H), 2.7-2.61 (m, 1H), 1.14 | | |
| | | (d, 3H), 0.84 (d, 3H) | | |
| 44 | B, A | 9.12-9.08 (m, 2H), 8.75 (d, 1H), 8.63 | 524.254383 | 524.2557 |
| | | (d, 1H), 8.20 (dd, 1H), 7.49-7.27 (m, | | |
| | | 8H), 7.24-7.18 (m, 3H), 6.83 (t, 1H), | | |
| | | 6.64 (d, 2H), 6.44 (s, 2H), 4.50 (d, | | |
| | | 2H), 3.95 (t, 2H), 2.96 (t, 2H) 2.71- | | |
| | | 2.64 (m, 1H), 1.09 (d, 6H) | | |
| 45 | F, A | 9.13-9.07 (m, 2H), 8.82 (d, 1H), 8.61 | 623.286411 | 623.2839 |
| | | (d, 1H), 8.19 (dd, 1H), 7.48-7.27 (m, | | |
| | | 8H), 7.25-7.14 (m, 3H), 6.92 (q, 1H), | | |
| | | 6.64 (d, 2H), 6.46-6.35 (m, 2H), 4.49 | | |
| | | (d, 2H), 4.19-4.03 (m, 2H), 4.02-3.92 | | |
| | | (m, 4H), 3.39 (t, 1H), 2.96 (t, 2H), | | |
| | | 1.97 (d, 3H), 1.92-1.71 (m, 4H) | | |
| 46 | F, A | 9.12-9.07 (m, 2H), 8.80 (s, 1H), 8.61 | 581.275847 | 581.2849 |
| | | (d, 1H), 8.19 (dd, 1H), 7.49-7.27 (m, | | |
| | | 8H), 7.25-7.18 (m, 3H), 6.90 (t, 1H), | | |
| | | 6.64 (d, 2H), 6.46-6.33 (m, 2H), 4.50 | | |
| | | (d, 2H), 3.95 (t, 2H), 3.46-3.21 (m, | | |
| | | 3H), 3.13-3.03 (m, 2H), 2.96 (t, 2H), | | |
| | | 1.92-1.68 (m, 4H) | | |
| 47 | C, A | 9.22-9.19 (m, 2H), 8.75 (s, 1H), 8.59 | 538.270033 | 538.2716 |
| | | (d, 1H), 8.20 (dd, 1H), 7.49-7.28 (m, | | |
| | | 8H), 7.24-7.18 (m, 3H), 7.06 (q, 1H), | | |
| | | 6.86 (t, 1H), 6.64 (d, 2H), 4.50 (d, | | |
| | | 2H), 3.95 (t, 2H), 2.96 (t, 2H), 2.70- | | |
| | | 2.65 (m, 1H), 1.87 (d, 3H), 1.10 (d, | | |
| | | 3H), 1.06 (d, 3H) | | |
| 48 | D, A | 9.11 (s, 1H), 9.06 (d, 1H), 8.77 (s, | 569.239461 | 569.243 |
| | | 1H), 8.62 (d, 1H), 8.38 (t, 1H), 8.20 | | |
| | | (dd, 1H), 7.49-7.27 (m, 8H), 7.25- | | |
| | | 7.18 (m, 3H), 6.87 (t, 1H), 6.64 (d, | | |
| | | 2H), 6.41 (s, 2H), 4.49 (d, 2H), 3.95 | | |
| | | (t, 2H), 3.80 (d, 2H), 3.61 (s, 3H), | | |
| | | 2.96 (t, 2H) | | |
| 49 | D, A | 9.08 (s, 1H), 9.04 (d, 1H), 8.80 (s, | 583.255111 | 583.2601 |
| | | 1H), 8.61 (d, 1H), 8.19 (dd, 1H), 8.01 | | |
| | | (t, 1H), 7.49-7.27 (m, 8H), 7.25-7.18 | | |
| | | (m, 3H), 6.90 (t, 1H), 6.64 (d, 2H), | | |
| | | 6.37 (s, 2H), 4.49 (d, 2H), 3.95 (t, | | |
| | | 2H), 3.55 (s, 3H), 3.23 (q, 2H), 2.96 | | |
| | | (t, 2H), 2.47 (t, 2H) | | |
| 50 | D, A | 9.16-9.02 (m, 2H), 8.82 (s, 1H), 8.61 | 583.255111 | 583.2593 |
| | | (d, 1H), 8.20 (t, 1H), 7.49-7.27 (m, | | |
| | | 8H), 7.25-7.18 (m, 3H), 6.91 (t, 1H), | | |
| | | 6.64 (d, 2H), 6.42 (d, 2H), 4.49 (d, | | |
| | | 2H), 4.12 (d, 2H), 3.95 (t, 2H), 3.64 | | |
| | | (d, 3H), 2.96 (t, 2H), 2.91 (d, 3H) | | |
| 51 | A | 9.04 (s, 1H), 9.01 (d, 1H), 8.72 (s, | 468.228168 | 468.2298 |
| | | 1H), 8.60 (d, 1H), 8.19 (dd, 1H), | | |
| | | 7.49-7.27 (m, 8H), 7.25-7.18 (m, 3H), | | |
| | | 6.83 (t, 1H), 6.64 (d, 2H), 5.89 (s, | | |
| | | 2H), 4.50 (d, 2H), 3.95 (t, 2H), 3.40 | | |
| | | (s, 3H), 2.96 (t, 2H) | | |
| 52 | D, A | 9.13-8.99 (m, 2H), 8.79 (d, 1H), 8.62 | 609.270761 | 609.2715 |
| | | (d, 1H), 8.20 (dd, 1H), 7.49-7.27 (m, | | |
| | | 8H), 7.25-7.17 (m, 3H), 6.89 (q, 1H), | | |
| | | 6.64 (d, 2H), 6.50-6.34 (m, 2H), 4.50 | | |
| | | (d, 2H), 3.95 (t, 2H), 3.62 (d, 3H), | | |
| | | 3.51-3.34 (m, 2H), 3.08 (bs, 1H), 2.96 | | |
| | | (t, 2H), 2.28-2.11 (m, 1H), 1.99-1.69 | | |
| | | (m, 3H) | | |
| 53 | B, A | 9.26 (s, 1H), 9.23 (d, 1H), 9.17 (s, | 559.233982 | 559.2328 |
| | | 1H), 8.87 (d, 1H), 8.81 (s, 1H), 8.64 | | |
| | | (d, 1H), 8.35 (d, 1H), 8.23 (dd, 1H), | | |
| | | 7.61 (dd, 1H), 7.49-7.27 (m, 8H), | | |
| | | 7.24-7.18 (m, 3H), 6.91 (t, 1H), 6.71 | | |
| | | (s, 2H), 6.63 (d, 2H), 4.51 (d, 2H), | | |
| | | 3.95 (t, 2H), 2.96 (t, 2H) | | |
| 54 | B, A | 9.12 (s, 1H), 9.07 (d, 1), 8.76 (s, 1H), | 568.244212 | 568.2411 |
| | | 8.62 (d, 1H), 8.20 (dd, 1H), 7.49-7.27 | | |
| | | (m, 9H), 7.25-7.18 (m, 3H), 6.86 (t, | | |
| | | 1H), 6.64 (d, 2H), 6.46 (s, 2H), 4.49 | | |
| | | (d, 2H), 3.95 (t, 2H), 2.96 (t, 2H), | | |
| | | 2.70 (q, 2H), 2.60 (t, 2H) | | |
| 55 | B, A | 10.23 (s, 1H), 9.24 (s, 1H), 9.21 (d, | 574.233648 | 574.2336 |
| | | 1H), 8.77 (s, 1H), 8.64 (d, 1H), 8.23 | | |
| | | (dd, 1H), 7.80 (dd, 1H), 7.55-7.28 (m, | | |
| | | 10H), 7.24-7.18 (m, 3H), 7.01 (d, | | |
| | | 1H), 6.92 (t, 1H), 6.87 (t, 1H), 6.66 (s, | | |
| | | 2H), 6.63 (d, 2H), 4.51 (d, 2H), 3.95 | | |
| | | (t, 2H), 2.96 (t, 2H) | | |
| 56 | D, A | 9.13-9.09 (m, 2H), 8.96 (s, 1H), 8.62 | 553.280932 | 553.2794 |
| | | (d, 1H), 8.19 (dd, 1H), 7.49-7.27 (m, | | |
| | | 8H), 7.25-7.18 (m, 3H), 7.05 (t, 1H), | | |
| | | 6.64 (d, 2H), 6.40 (s, 2H), 4.50 (d, | | |
| | | 2H), 3.95 (t, 2H), 3.26 (q, 2H), 3.20 | | |
| | | (q, 2H), 2.96 (t, 2H), 1.08-0.99 (m, | | |
| | | 6H) | | |
| 57 | D, A | 9.13-9.09 (m, 2H), 8.80 (s, 1H), 8.61 | 609.307147 | 609.3086 |
| | | (d, 1H), 8.19 (dd, 1H), 7.49-7.27 (m, | | |
| | | 8H), 7.25-7.18 (m, 3H), 6.89 (bs, 1H), | | |
| | | 6.64 (d, 2H), 6.47-6.33 (m, 2H), 4.49 | | |
| | | (d, 2H), 4.42 (t, H), 3.95 (t, 2H), 3,73- | | |
| | | 3.43 (m, 2H), 2.96 (t, 2H), 1.99-1.64 | | |
| | | (m, 4H), 1.02 (s, 3H), 0.99 (s, 3H) | | |
| 58 | D, A | 9.08 (s, 1H), 9.05 (d, 1H), 8.73 (s, | 555.260197 | 555.2606 |
| | | 1H), 8.61 (d, 1H), 8.19 (dd, 1H), 7.98 | | |
| | | (t, 1H), 7.49-7.27 (m, 8H), 7.25-7.18 | | |
| | | (m, 3H), 6.83 (t, 1H), 6.64 (d, 2H), | | |
| | | 6.37 (s, 2H), 4.49 (d, 2H), 3.95 (t, | | |
| | | 2H), 3.32 (t, 2H), 3.19 (s, 3H), 3.14 | | |
| | | (q, 2H), 2.96 (t, 2H) | | |
| 59 | D, A | 9.12-9.07 (m, 2H), 8.87 (s, 1H), 8.61 | 551.265282 | 551.2675 |
| | | (d, 1H), 8.18 (dd, 1H), 7.49-7.27 (m, | | |
| | | 8H), 7.24-7.18 (m, 3H), 6.96 (t, 1H), | | |
| | | 6.64 (d, 2H), 6.40 (s, 2H), 4.50 (d, | | |
| | | 2H), 3.95 (t, 2H), 3.35 (t, 2H), 3.24 (t, | | |
| | | 2H), 2.96 (t, 2H), 1.82-1.71 (m, 4H) | | |
| 60 | D, A | 9.12 (s, 1H), 9.11 (d, 1H), 8.89 (s, | 567.260197 | 567.2679 |
| | | 1H), 8.61 (d, 1H), 8.19 (dd, 1H), | | |
| | | 7.49-7.27 (m, 8H), 7.24-7.18 (m, 3H), | | |
| | | 6.98 (t, 1H), 6.64 (d, 2H), 6.42 (s, | | |
| | | 2H), 4.50 (d, 2H), 3.95 (t, 2H), 3.58- | | |
| | | 3.52 (m, 4H), 3.46-3.41 (m, 2H), | | |
| | | 3.35-3.29 (m, 2H), 2.96 (t, 2H) | | |

TABLE 2

| 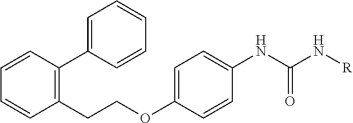 | | Prodrugs of Parent Compound A | | Stability Assessments | |
|---|---|---|---|---|---|
| | | Registered | | Stability at | Stability at |
| Ex. | | purity | | 40° C. and | 60° C. and |
| Number | R | (%) | Crystalline | 75% RH | 75% RH |
| 34 | 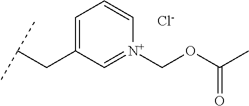 | ND | Oiled out upon collection at room temperature | ND | ND |
| 35 | 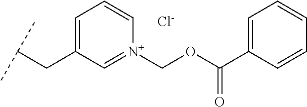 | 98, 95, 92, 96 | DSC 177.53° C. | No change in morphology stopped at 13 days, only prodrug observed | Deliquesced at 7 days, approx 30:70 parent to prodrug observed |
| 36 | 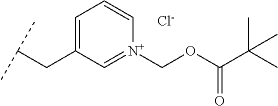 | ND | DSC 142.79° C. | ND | Oiled out at 1 hr No change in composition |
| 37 | 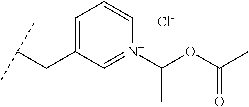 | 73 (>90 NMR) | DSC polymorphic 141.71, 180.41, 266.37° C. | Deliquesced at 2 Hr No change in composition | Deliquesced at 2 Hr No change in composition |
| 38 | 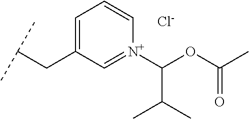 | 97, 85 (>95 NMR) | DSC 176.17° C. | No change in morphology at 7 days | Deliquesced at 4 days |
| 39 | 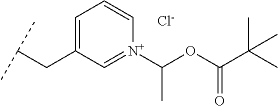 | ND | DSC 124.77° C. | Deliquesced at 2 Hr No change in composition | Deliquesced at 2 Hr No change in composition |
| 40 | 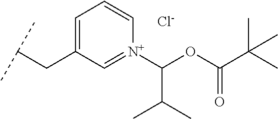 | ND | DSC 196.97° C. | No change in morphology at 14 days, only prodrug observed | Approximately 5:95 parent to prodrug moiety observed at 14 days |
| 41 | 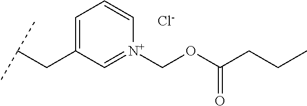 | 97 | DSC 170.56° C. | No change in morphology at 14 days, only prodrug observed | Deliquesced at 24 hr No change in composition |
| 42 | 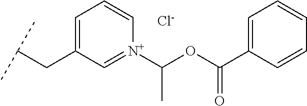 | 93 | DSC 168.01° C. | No change in morphology at 14 days, only prodrug observed | Deliquesced at 13 days, approx 40:60 parent to prodrug observed |
| 43 | 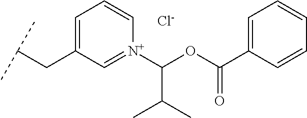 | 58 (~80 NMR) | DSC 196.65° C. | No change in morphology at 14 days, only prodrug observed | Approximately 5:95 parent to prodrug observed at 14 days |
| 44 | 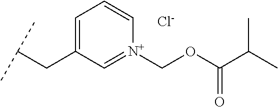 | 99+, 97 | DSC 172.78° C. | No change in morphology at 14 days, circa 7:93 parent to prodrug | Deliquesced at 5 days approximately 50:50 parent to prodrug |
| 45 | 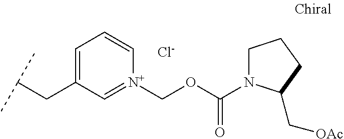 | 73, 92 (>90 NMR) | Crystalline material not obtained | ND | ND |
| 46 | 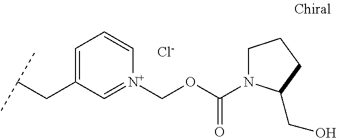 | 77 (>90 NMR) | Crystalline material not obtained | ND | ND |
| 47 | 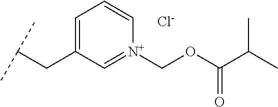 | 76 (>90% NMR) | DSC 181.43° C. | No change in morphology at 14 days, only prodrug observed | Approximately 45:55 parent to prodrug observed after 14 days |
| 48 | 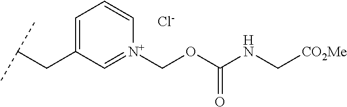 | 88 (>95 NMR) | DSC polymorphic 129.84, 137.53, 179.36° C. | Deliquesced at 2 hr No decomposition observed | Deliquesced at 2 hr No decomposition observed |
| 49 | 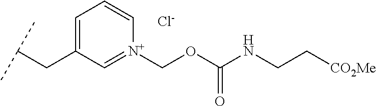 | 91 (>95 NMR) | DSC polymorphic 125.69, 188.04° C. | Deliquesced at 24 hrs No decomposition observed | Deliquesced at 7 days |
| 50 | 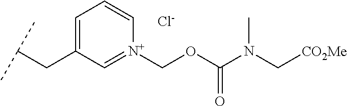 | 99 | DSC 147.12° C. | No change in morphology at 14 days, only prodrug observed | Oiled out at 2 days No decomposition observed |
| 51 | 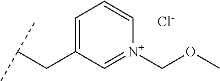 | 99 | Poor crystals | ND | ND |
| 52 | 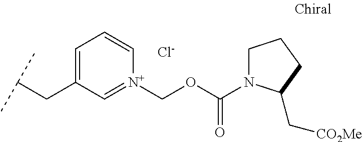 | 87 | ND | ND | ND |
| 53 | 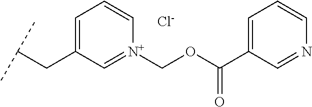 | 80 | DSC 150.89° C. | ND | All prodrug decomposed to parent at 24 h |
| 54 | 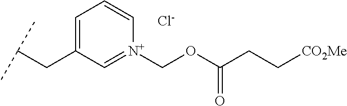 | 98 | DSC 159.93° C. | ND | Deliquesced at 4 days |
| 55 | 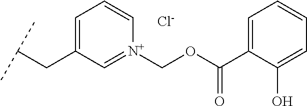 | 90 | DSC 152.54° C. | Deliquesced at 4 h | Deliquesced at 4 h |
| 56 | 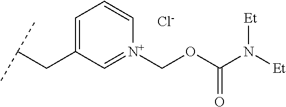 | 99 | DSC 153.74° C. | ND | ND |
| 57 | 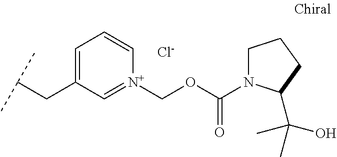 | 99 | ND | ND | ND |
| 58 | 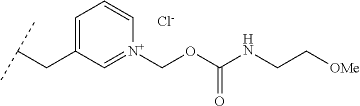 | 95 | ND | ND | ND |
| 59 | 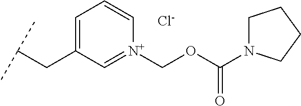 | 95 | DSC 159.63° C. | ND | Deliquesced at 4 h |
| 60 | 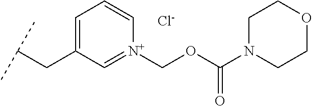 | 96 | DSC Unknown ° C. | Deliquesced at 4 h | Deliquesced at 4 h |

Table 2 list physical chemical data for each of Example Compounds 34 through 60. “ND” in Table 2 refers to “No Data”. Purity was determined by HPLC. Melting point was determined by differential scanning calorimetry (“DSC”), and semi-quantitative hygroscopicity was determined in a relative humidity (“RH”) chamber.

### Biochemical and Biological Examples

### Kinetic Solubility Assays

Kinetic solubility was determined using the iSol Explorer method (pION Inc.; Woburn, Mass.). Certain Example Compounds were dissolved in dimethylsulfoxide (DMSO; >99.9% ACS spectrophotometric grade; Sigma-Aldrich, St. Louis, Mo.) to a concentration of 100 mM. The DMSO stock was diluted 1:100 in system solution buffer (pION Inc. Wobum Mass.) that had been adjusted to the desired pH (5.0, 6.8, and 7.4) with either HCl or NaOH. The diluted samples in the desired buffer were incubated for 15 minutes at room temperature and filtered using 96-well plate with 0.25 mm glass fiber/1.2 μm polyethersulfone filter bottoms (Corning, Inc.; Corning, N.Y.). The filtrate is transferred to a second 96-well plate and read on a SpectraMAX 190 (Molecular Devices; Sunnyvale, Calif.) scanning from 190 to 498 nm. The data are analyzed with Sol Explorer Command version 2.2, (pION Inc. Woburn, Mass.) and the concentration reported in molarity and/or mass per volume. The results of these assays are shown in Tables 3A, 3B, and 3C below.

### Stabilities in Plasma and Liver Microsome Preparations

Relative stability of Example Compound Nos. 1 through 60 in mouse plasma and mouse liver microsomal preparations.

Certain Example Compounds were each added to a final concentration of 1 μM to 1 mL of each of mouse plasma and mouse liver microsomal preparations (with and without 1 mM NADPH). Stability assays were run in duplicate and were sampled at 0, 5, 15, 30, 60 and 120 minutes as follows: one hundred microliter aliquots of samples were removed at the specified sampling times, and the reaction was quenched with 400 μL of acetonitrile.

Samples were analyzed by HPLC-ESI-MS/MS by measuring the peak area of both the starting Example Compound and parent compound (i.e., either Parent Compound A or Parent Compound B) that were created by removal of the Prodrug Moiety (as defined in Section 6 herein). The results of these studies are shown in Tables 3A, 3B, and 3C below.

### Tables 3A, 3B and 3C

TABLE 3A
Solubility & Stability of Certain Acyloxyalkyl Example Compounds

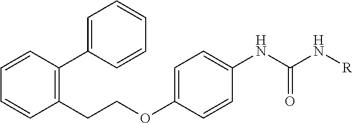

| | | Solubility | Mouse | Mouse |
|---|---|---|---|---|
| | | (μM) | Liver | Plasma |
| Example | | pH 5.0, 6.8, | Microsomes | t_{1/2} |
| No. | R | 7.4 | t_{1/2} min | min |
| 34 | 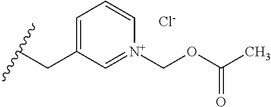 [SMILES: *Cc1ccc[n+](COC(C)=O)c1 ; [Cl-]] | 396.69, 393.12, 398.69 | <5 | |
| 36 | 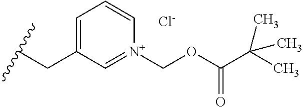 [SMILES: *Cc1ccc[n+](COC(=O)C(C)(C)C)c1 ; [Cl-]] | 600.88, 546.53, 523.98 | | |
| 37 | 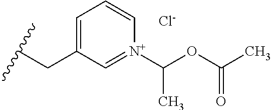 [SMILES: *Cc1ccc[n+](C(C)OC(C)=O)c1 ; [Cl-]] | | 18.7 | >5 |
| 38 | 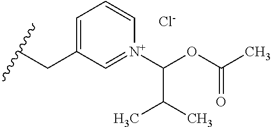 [SMILES: *Cc1ccc[n+](C(OC(C)=O)C(C)C)c1 ; [Cl-]] | | 30.9 | >5 |
| 39 | 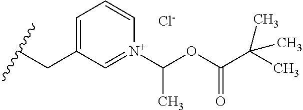 [SMILES: *Cc1ccc[n+](C(C)OC(=O)C(C)(C)C)c1 ; [Cl-]] | | 112.8 | 27 |
| 40 | 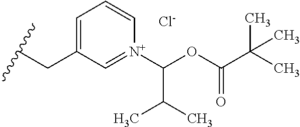 [SMILES: *Cc1ccc[n+](C(OC(=O)C(C)(C)C)C(C)C)c1 ; [Cl-]] | | 399.2 | 53.48 |
| 41 | 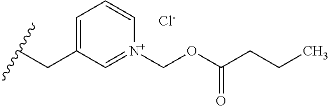 [SMILES: *Cc1ccc[n+](COC(=O)CCC)c1 ; [Cl-]] | | <5 | <5 |
| 44 | 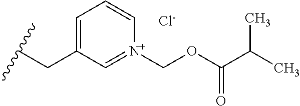 [SMILES: *Cc1ccc[n+](COC(=O)C(C)C)c1 ; [Cl-]] | | <5 | <5 |
| 47 | 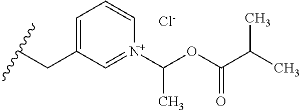 [SMILES: *Cc1ccc[n+](C(C)OC(=O)C(C)C)c1 ; [Cl-]] | | | |

TABLE 3B
Solubility and Stability of Certain Benzoyloxyalkyl Example Compounds

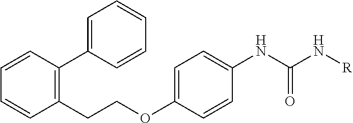

| | | Solubility | Mouse | Mouse |
|---|---|---|---|---|
| | | (μM) | Liver | Plasma |
| Example | | pH 5.0, 6.8, | Microsomes | t_{1/2} |
| No. | R | 7.4 | t_{1/2} min | min |
| 35 | 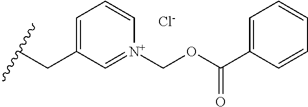 [SMILES: *Cc1ccc[n+](COC(=O)c2ccccc2)c1 ; [Cl-]] | 401.5, 403.5, 433.0 | <5 | 77.18 |
| 42 | 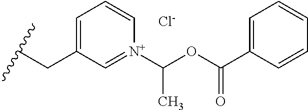 [SMILES: *Cc1ccc[n+](C(C)OC(=O)c2ccccc2)c1 ; [Cl-]] | | | |
| 43 | 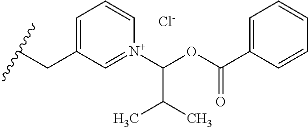 [SMILES: *Cc1ccc[n+](C(OC(=O)c2ccccc2)C(C)C)c1 ; [Cl-]] | | 59.1 | 29.84 |

TABLE 3C
Solubility and Stability of Certain Carbamoyloxyalkyl Example Compounds

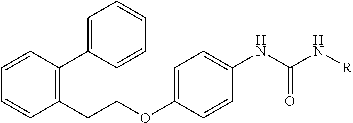

| | | Solubility | Mouse | Mouse |
|---|---|---|---|---|
| | | (μM) | Liver | Plasma |
| Example | | pH 5.0, 6.8, | Microsomes | t_{1/2} |
| No. | R | 7.4 | t_{1/2} min | min |
| 45 | 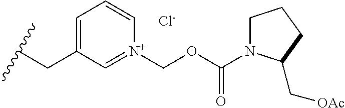 [SMILES: *Cc1ccc[n+](COC(=O)N2CCC[C@H]2COC(C)=O)c1 ; [Cl-]] | | >5 | >5 |
| 46 | 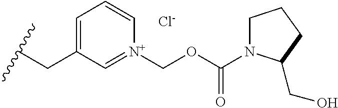 [SMILES: *Cc1ccc[n+](COC(=O)N2CCC[C@H]2CO)c1 ; [Cl-]] | | 1246 | 87.2 |
| 48 | 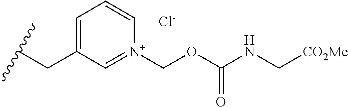 [SMILES: *Cc1ccc[n+](COC(=O)NCC(=O)OC)c1 ; [Cl-]] | | | |
| 49 | 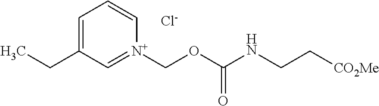 [SMILES: CCc1ccc[n+](COC(=O)NCCC(=O)OC)c1 ; [Cl-]] | | | |
| 50 | 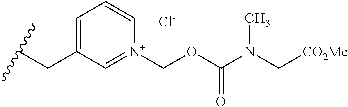 [SMILES: *Cc1ccc[n+](COC(=O)N(C)CC(=O)OC)c1 ; [Cl-]] | | | |
| 51 | 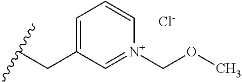 [SMILES: *Cc1ccc[n+](COC)c1 ; [Cl-]] | | | |

### Pharmacokinetic Studies of Certain Example Compounds

Oral bioavailability relative to intravenous administration of Parent Compound A was determined for certain Example Compounds. The results are depicted in Tables 4A, 4B, and 4C.

All pharmacokinetic studies of Example Compounds were conducted in either CD-1 or NuHet female mice. Plasma concentrations of Parent Compound A were quantified by LC-ESI-MS/MS using an internal standard and calibration curve with quality control samples. Animals were dosed by oral gavage with the Example Compound formulated in one of two different vehicles. To determine pharmacokinetic properties of Parent Compound A, Parent Compound A was administered intravenously to mice or rats.

The basic pharmacokinetics (Cmax, Tmax, AUC, half-life and % F) of the Example Compounds was determined in female CD-1 mice or female Sprague Dawley rats dosed at 5 to 30 mg/kg orally. The dosing vehicle consisted of one of the following: 50% of 5% dextrose in water and 50% of 0.5% hydroxy propyl methyl cellulose plus 0.05% Tween-80 or 0.5% hydroxyl propyl methyl cellulose, 50 mm Glycine buffer pH 3.0 plus 0.05% tween-80. The dosing vehicle for intravenous administration of Parent Compound A was 3% N,N-dimethylacetamide, 40% polyethylene glycol 300, 12% ethanol, 45% water mixed volume by volume. Three or five animals were used per time point and the time-points collected were 15, 30, 60, 120, 240, and 480 minutes post-administration for oral dosing, and 5, 15, 30, 60, 120, 240 and 480 minutes post-administration for intravenous dosing. Blood samples were collected through cardiac puncture and plasma obtained with EDTA as the anticoagulant. Plasma was submitted for bioanalysis. Concentrations of Parent Compound A were determined by adding 10 μL of internal standard to 50 μL of sample and then precipitating protein with 400 μL of acetonitrile. The supernatant was injected onto a reverse-phase HPLC column. Separation was achieved by gradient elution. Analytes were ionized by positive-ion electrospray ionization and detected by multiple-reaction monitoring on an ABI Q-Trap. Peak area ratios of the Example Compounds and Parent Compound A and internal standard were compared to those from a multi-point standard curve spanning a range from 10 to 10,000 ng/mL. To confirm accuracy, quality control samples were analyzed in duplicate at 4 different concentrations. Bioanalytical run acceptance required that at least three-fourths of the calibration standards were within 15% of theoretical and that greater than ⅔ of all quality control samples were within 25% of theoretical. Plasma concentration data was fit in WinNonLin® (v. 5.1.1) using noncompartmental analysis.

### Tables 4A, 4B and 4C

TABLE 4A
Pharmacokinetics of Certain Acyloxyalkyl Example Compounds

| Exam- | Oral | | | | | | |
|---|---|---|---|---|---|---|---|
| ple | Dose | | t_{1/2} | Tmax | Cmax | AUC | |
| No. | mg/Kg | Species | (hr) | (hr) | (ng/mL) | (hr*ng/mL) | % F |
| 34 | 10.0 | Mouse | .8 | .5 | 591 | 885 | 25 |
| 36 | 10.0 | Mouse | 1.3 | .5 | 364 | 354 | 10 |
| 37 | 10.0 | Mouse | 2.2 | 1 | 488 | 798 | 29.1 |
| 38 | 10.0 | Mouse | 3.2 | 1 | 336 | 758 | 21.9 |
| 39 | 10.0 | Mouse | 1.3 | .25 | 436 | 555 | 17.5 |
| 40 | 10.0 | Mouse | 1.6 | .25 | 267 | 219 | 8.7 |
| 41 | 10.0 | Mouse | 1.7 | .5 | 145 | 153 | 12.8 |
| 44 | 10.0 | Mouse | 1.5 | .25 | 550 | 596 | 16.5 |
| 47 | 10.0 | Mouse | 1.5 | .25 | 268 | 449 | 16.6 |

TABLE 4B
Pharmacokinetics of Certain Benzoyloxyalkyl
Example Compounds

| Exam- | Oral | | | | | | |
|---|---|---|---|---|---|---|---|
| ple | Dose | | t_{1/2} | Tmax | Cmax | AUC | |
| No. | mg/Kg | Species | (hr) | (hr) | (ng/mL) | (hr*ng/mL) | % F |
| 35 | 5.0 | Mouse | 1.9 | .5 | 74 | 225 | 13.3 |
| 35 | 10.0 | Rat | 1.2 | 4 | 258 | 977 | 23.6 |
| 35 | 30.0 | Mouse | 7.3 | .25 | 2340 | 6086 | 54.4 |
| 35 | 20.0 | Rat | 1 | 1 | 2819 | 7014 | 90.2 |
| 35 | 10.0 | Mouse | 1.8 | 1 | 432 | 825 | 24.2 |
| 42 | 10.0 | Mouse | 1.3 | .25 | 353 | 417 | 13.2 |
| 43 | 10.0 | Mouse | 1.2 | .25 | 843 | 721 | 38.6 |

TABLE 4C
Pharmacokinetics of Certain Carbamoyloxyalkyl
and other Example Compounds

| Exam- | Oral | | | | | | |
|---|---|---|---|---|---|---|---|
| ple | Dose | | t_{1/2} | Tmax | Cmax | AUC | |
| No. | mg/Kg | Species | (hr) | (hr) | (ng/mL) | (hr*ng/mL) | % F |
| 45 | 10.0 | Rat | 1.5 | 2 | 341 | 1383 | 49.7 |
| 45 | 10.0 | Mouse | 2.4 | 1 | 452 | 569 | 25.1 |
| 46 | 10.0 | Mouse | 1.1 | .5 | 203 | 458 | 17.9 |

Anti-Tumor Activity of Parent Compound A in HT1080 Human Fibrosarcoma Xenograft with Twice Daily Dosing

The objective of this study was to determine the effect of Parent Compound A administration on the growth of HT1080 human fibrosarcoma cancer cells as a xenograft in athymic nude mice, when given on a twice daily dosing schedule.

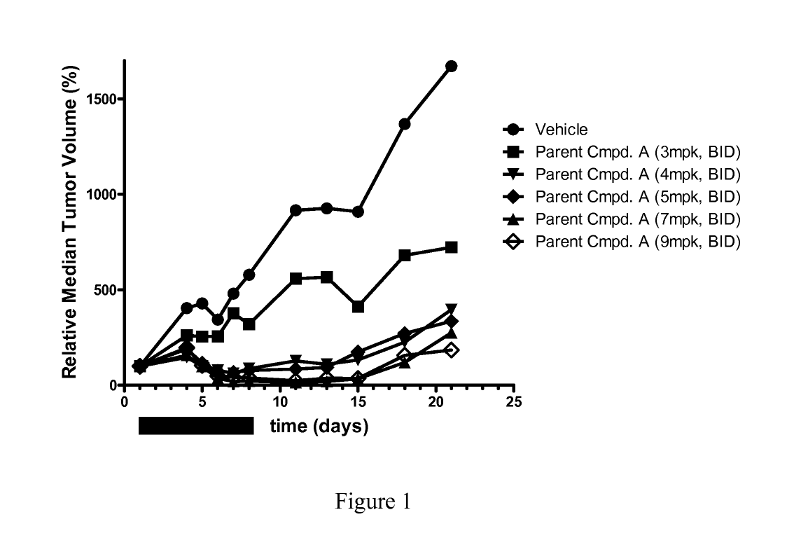

One million HT1080 cells were implanted subcutaneously in the right flank of female nude mice (Crl:NU-Fox1^{nu}). When the median tumor volume (MTV) was approximately 100 mm³, mice were randomized into six cohorts of ten animals. Cohort one was administered vehicle orally (30% Captisol®) and cohorts 2 to 6 were administered orally Parent Compound A at 3, 4, 5, 7 or 9 mg/kg, respectively, twice daily for seven days. The mice were observed daily for mortality and signs of toxicity. Tumor volumes and body weights were determined from Days 1 to 21. The results are depicted in FIG. 1.

MTV of the cohorts administered vehicle and Parent Compound A at 3 mg/kg increased by 479%, and 221% by the end of dosing on study Day 9, respectively. In contrast, MTV of the cohorts dosed with Parent Compound A at 4, 5, 7 and 9 mg/kg were 87%, 78%, 22% and 39% of the initial value, respectively over the same period. These data indicate that Parent Compound A administered twice daily at 4, 5, 7 or 9 mg/kg induced tumor regressions of 13% (p=0.01), 22% (p=0.0003), 78% (p=0.0002) and 61% (p<0.0001), respectively by Day 8.

MTV of the cohorts dosed with vehicle or Parent Compound A at 3, 4, 5, 7 or 9 mg/kg increased by 1572%, 622%, 296%, 235%, 175%, and 85% respectively, at the end of the study on Day 21. These data indicate that Parent Compound A administered at 3, 4, 5, 7, 9 mg/kg resulted in tumor growth inhibition relative to vehicle of 60% (p=0.7), 81% (p=0.09), 85% (p=0.02), 89% (p=0.01) and 95% (p=0.01), respectively on Day 21, fourteen days after dosing was stopped.

Parent Compound A was well tolerated with a maximal reduction in median body weight of <10% of the initial weight over the course of the study. Three animals in the vehicle group and three animals in the group dosed with Parent Compound A at 3 mg/kg had tumor volumes exceeding 1,500 mm³ and were euthanized. There were three deaths in the group dosed with Parent Compound A at 4 mg/kg, two animals were found dead and one had tumor volume exceeding 1,500 mm³ and was euthanized. One animal in each of the groups dosed with Parent Compound A at 5, 7, or 9 mg/kg had no detectable tumor by the end of the study.

Parent Compound A administered orally, twice daily at 3, 4, 5, 7 or 9 mg/kg was well tolerated and showed significant anti-tumor activity in the HT1080 xenograft model, with doses at or above 4 mg/kg causing tumor regressions at the end of dosing.

Anti-Tumor Activity of Example Compound 35 in HT1080 Human Fibrosarcoma Xenograft with Twice Daily Dosing

The objective of this study was to determine the effect of Example Compound 35 administration on the growth of HT1080 human fibrosarcoma cancer cells as a xenograft in athymic nude mice, when given on a twice daily dosing schedule.

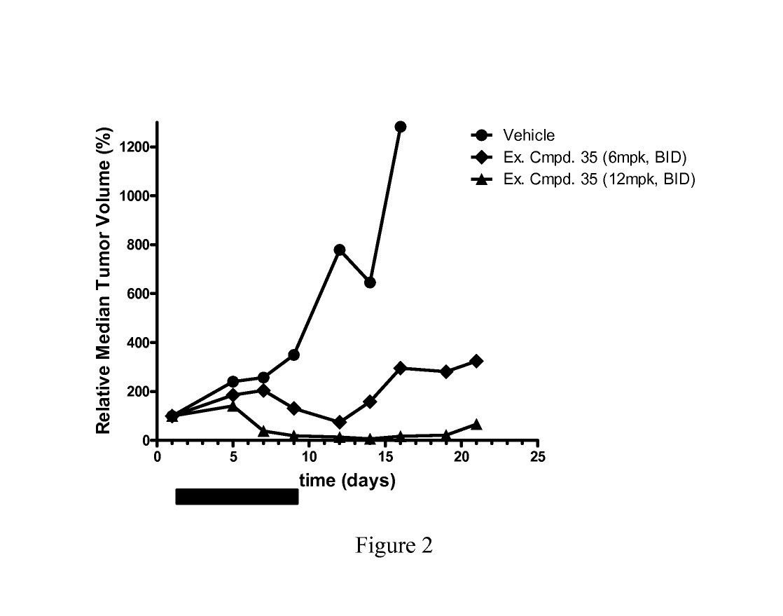

One million HT1080 cells were implanted subcutaneously in the right flank of female nude mice (Crl:NU-Fox1^{nu}). When the median tumor volume (MTV) was approximately 82 mm³, mice were randomized into three cohorts of ten animals. One cohort was dosed with vehicle (5% Dextrose in water with 0.1% Tween 80), a second with Example Compound 35 (6 mg/kg), and the third with Example Compound 35 (12 mg/kg). Example Compound 35 was formulated as an aqueous suspension in 50 mM Glycine, pH 3.0, with 0.5% hydroxypropyl methylcellulose (HPMC), and 0.05% Tween-80, and administered twice daily for 8 days. The mice were observed daily for mortality and signs of toxicity. Tumor volumes and body weights were determined from Days 1 to 21. The results are depicted in FIG. 2.

MTV of the cohort dosed with vehicle increased by 250%, and 1027% by the end of dosing on study Day 9 and the end of the study on Day 21, respectively. In contrast, MTV of the cohort dosed with Example Compound 35 at 6 mg/kg increased by only 31% and 2245% by Day 9 and Day 21, respectively, and MTV of the cohort dosed at 12 mg/kg decreased by 81% and 33%, respectively. These data indicate that Example Compound 35 dosed twice daily at 6 mg/kg inhibited tumor growth of HT1080 xenografts relative to vehicle by 87% on Day 9 (p=0.004) and 78% on Day 21 (p=0.06), and Example Compound 35 dosed at 12 mg/kg resulted in tumor regression of 80% on Day 9 (p=0.0001) and 33% (p=0.001) on Day 21. Example Compound 35 was well tolerated with a maximal reduction in median body weight of <15% of the initial weight over the course of the study. Three animals in the vehicle group had tumor volumes exceeding 1,500 mm³ and were euthanized. No animals receiving Example Compound 35 were euthanized. Four animals in the group dosed with Example Compound 35 at 12 mg/kg had no detectable tumors by the end of the study. Based on molecular weight of Example Compound 35 and Parent Compound A, 12 mg/kg of Example Compound 35 after removal of the Prodrug Moiety is roughly equivalent to 9 mg/kg of Parent Compound A.

Example Compound 35 administered orally, twice daily at 6 or 12 mg/kg was well tolerated and showed significant anti-tumor activity in the HT 1080 xenograft model with the higher dose causing tumor regressions.

Anti-Tumor Activity of Example Compound 42 in HT1080 Human Fibrosarcoma Xenograft with Twice Daily Dosing

The objective of this study was to determine the effect of administration of Example Compound 42 on the growth of HT1080 human fibrosarcoma cancer cells as a xenograft in athymic nude mice, when given on a twice daily dosing schedule.

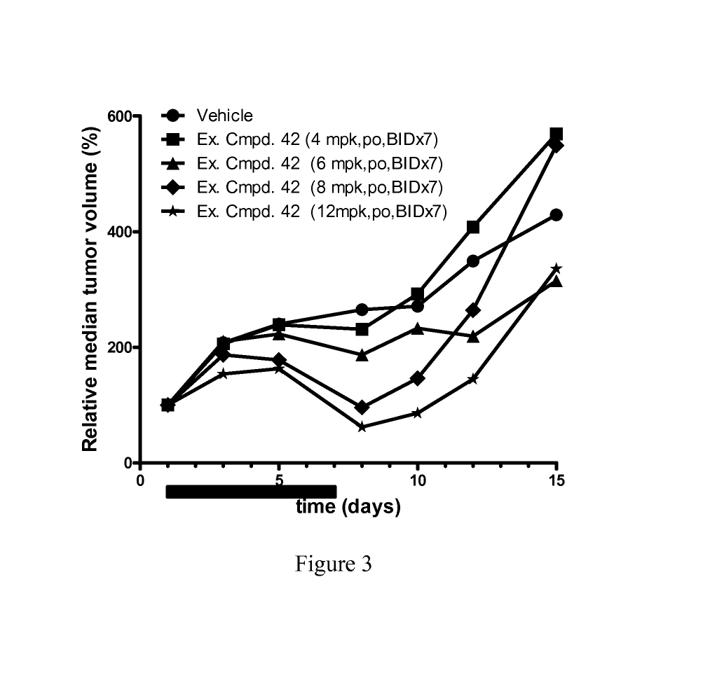

One million HT1080 cells were implanted subcutaneously in the right flank of female nude mice (Crl:NU-Fox1^{nu}). When the median tumor volume (MTV) was approximately 134 mm³, mice were randomized into five cohorts of ten animals. Cohort 1 was dosed with vehicle (50 mM Glycine, pH 3.0, with 0.5% hydroxypropyl methylcellulose (HPMC), and 0.05% Tween-80); cohorts 2, 3, 4 and 5 with Example Compound 42 at 4, 6, 8 or 12 mg/kg twice daily for 7 days. The mice were observed daily for mortality and signs of toxicity. Tumor volumes and body weights were determined from Days 1 to 15. The results are depicted in FIG. 3.

MTV of the cohort dosed with vehicle increased by 165% by the end of dosing on study Day 8. In contrast, MTV of the cohorts dosed with Example Compound 42 at 4 and 6 mg/kg increased by 131% and 87% respectively on Day 8, and MTV of the cohorts dosed at 8 or 12 mg/kg decreased by 4% and 38%, respectively. These data indicated that Example Compound 42 dosed at 4, 6, 8 and 12 mg/kg resulted in tumor growth inhibition of 21% (p>0.05) and 48% (p>0.05) or tumor regression of 4% (p<0.0078) or 38% (p<0.0003), respectively at the end of dosing on Day 8. The effect of Example Compound 42 on tumor growth was not sustained and the MTV of the cohorts dosed with vehicle or Example Compound 42 were similar at the end of the study on Day 15, with increases of 329%, 469%, 215%, 449% and 236%, respectively. One animal died in each of the groups dosed with Example Compound 42 at 6, 8 and 12 mg/kg. The groups dosed with vehicle only had one animal euthanized due to tumor volume exceeding 1,500 mm³. Example Compound 42 at 4, 6 or 8 mg/kg had three, one, and two animals euthanized, respectively, due to tumor volume exceeding 1,500 mm³.

Example Compound 42 administered orally, twice daily showed dose-dependent anti-tumor activity in the HT1080 xenograft model and induced tumor regression at the end of dosing, when dosed at 8 or 12 mg/kg.

Anti-Tumor Activity of Example Compounds 44 and 45 in HT1080 Human Fibrosarcoma Xenograft with Twice Daily Dosing

The objective of this study was to determine the effect of administration of Example Compounds 44 or 45 on the growth of HT1080 human fibrosarcoma cancer cells as a xenograft in athymic nude mice, when given on a twice daily dosing schedule.

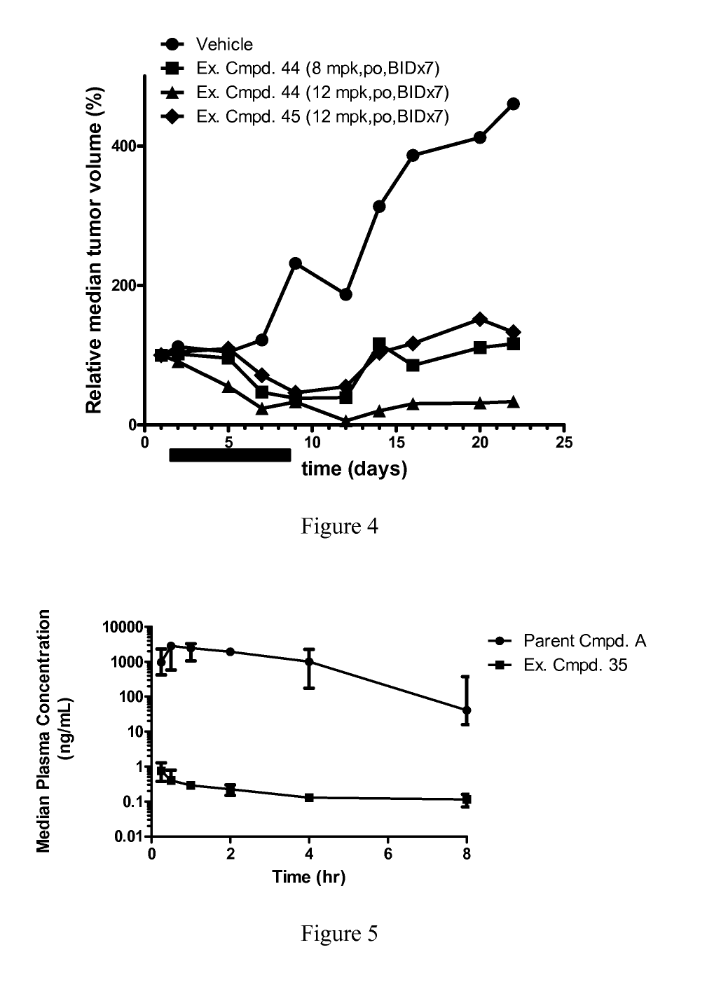

One million HT1080 cells were implanted subcutaneously in the right flank of female nude mice (Crl:NU-Fox1^{nu}). When the median tumor volume (MTV) was approximately 209 mm³, mice were randomized into four cohorts of ten animals. Cohort 1 was dosed with vehicle (50 mM Glycine, pH 3.0, with 0.5% hydroxypropyl methylcellulose (HPMC), and 0.05% Tween-80); cohorts 2 and 3 with Example Compound 44 at 8 and 12 mg/kg, respectively; and cohort 4 with Example Compound 45 at 12 mg/kg. All cohorts were dosed twice daily for 7 days. The mice were observed daily for mortality and signs of toxicity. Tumor volumes and body weights were determined from Days 1 to 22. The results are depicted in FIG. 4.

MTV of the cohort dosed with vehicle increased by 132% by the end of dosing on study Day 9. In contrast, MTV of the cohorts dosed with Example Compound 44 at 8 or 12 mg/kg and of Example Compound 45 dosed at 12 mg/kg decreased by 62%, 67% and 54%, respectively on Day 9. Similarly, MTV of the vehicle cohort increased by 360% at the end of the study on Day 22, whereas MTV of the cohorts dosed with Example Compound 44 at 8 mg/kg and of Example Compound 45 dosed at 12 mg/kg increased by only 16% and 33% over the same period and MTV of the cohort dosed with Example Compound 44 at 12 mg/kg decreased by 67%. These data indicate that Example Compound 44 dosed twice daily at 8 or 12 mg/kg induced tumor regression by 62% (p<0.0001) and 67% (p=0.0002), respectively on Day 9 and inhibited tumor growth by 95% (p=0.0198) or caused tumor regression by 67% (p=0.0012), respectively at the end of the study. Whereas, Example Compound 45 (12 mg/kg) induced tumor regression by 54% (p=0.0001) on Day 9 and inhibited tumor growth relative to vehicle by 91% (p=0.037) on Day 22. Both Example Compounds were well tolerated with a maximal reduction in median body weight of <15% of the initial weight over the course of the study. Four animals in the vehicle group were euthanized; three had tumor volumes exceeding 1,500 mm³, and one had an ulcerated tumor. One animal in the group dosed with Example Compound 44 at 8 mg/kg and three in the group dosed at 12 mg/kg had no detectable tumors by the end of the study.

Example Compound 44 administered orally, twice daily at 8 or 12 mg/kg, and Example Compound 45 at 12 mg/kg were well tolerated and showed significant anti-tumor activity in the HT1080 xenograft model, causing >90% tumor growth inhibition or tumor regression.

### Determination of Example Compound 35 and Parent Compound A in Portal Vein Samples of Rats Following Oral Administration of Example Compound 35

It is believed that Example Compound 35 metabolizes in vivo into Parent Compound A (i.e., it is believed that Example Compound 35 is a prodrug of Parent Compound A). The objective of this study was to determine the concentration of Example Compound 35 and Parent Compound A in hepatic portal vein samples of female Sprague-Dawley rats following oral administration of only Example Compound 35, and to quantify the extent of transport into hepatic portal circulation, if any, of Example Compound 35. A second objective was to determine the relative bioavailability of Parent Compound A in the portal vein following oral administration of Example Compound 35.

Experiment 1. Three female Sprague Dawley rats (200-215 g, Harlan Laboratories, Indianapolis, Ind.) with cannulae implanted surgically in the portal vein for blood sampling were used in this study. Example Compound 35, formulated in 50 mM Glycine pH 3.0 with 0.5% HPMC and 0.05% Tween-80, was administered by oral gavage (PO) at a dose of 20 mg/kg in a volume of 2 mL/kg. This dose is the molar equivalent of a 13.5 mg/kg dose of Parent Compound A. Portal blood samples were collected through the portal vein cannula from each animal at 0.25, 0.5, 1, 2, 4, and 8 hours following administration of compound. Samples were split into two fractions and one was supplemented with citrate buffer (final concentration 100 mM, pH 3.0) to stabilize Example Compound 35 and stop conversion of Example Compound 35 to Parent Compound A ex vivo during analysis. Samples were then processed to generate plasma and analyzed for Example Compound 35 by LC-MS/MS. Plasma derived from the second aliquot of blood was analyzed for Parent Compound A.

Experiment 2. Three female Sprague Dawley rats (215-250 g, Charles River Laboratories) with a catheter implanted surgically in the femoral vein for blood sampling were used in this study. Parent Compound A, solubilized in 3% DMA, 40% PEG300, 12% ethanol, and 45% water by volume, was administered IV at a dose of 2.5 mg/kg in a volume of 2 mL/kg. Femoral blood samples were collected from each animal at 0.083, 0.25, 0.5, 1, 2, 4, and 8 hours following administration of compound and processed for analysis of Parent Compound A concentration.

Pharmacokinetic parameters estimated for Parent Compound A included the maximum observed drug concentration (Cₘₐₓ), time of maximum observed drug concentration (Tₘₐₓ), apparent terminal half-life (t_{1/2}), area under the concentration-time curve from time zero to 8 hours postdose (AUC₍₀₋₈₎), and area under the concentration-time curve extrapolated to infinity (AUC_{(0-inf)}). The bioavailability (% F) of Parent Compound A in portal vein samples relative to IV administration was also estimated.

Plasma samples were analyzed for Example Compound 35 or Parent Compound A concentration using a LC-MS/MS method. All sample runs met standard acceptance criteria for discovery purposes, and were based on a standard curve containing at least 7 points. At least half of quality control samples at each of the three concentrations tested, and two thirds of the entire quality control sample pool, were within 15% of their theoretical value. The r² value for each standard curve was ≧0.99. The quantitation range for the Example Compound 35 and Parent Compound A assays was 1 to 100 ng/mL and 5 to 2,500 ng/mL, respectively, using a 0.05 mL sample volume.

The data were entered into the PK software package WinNonlin® version 5.1.1. A complete data check was conducted to verify the accuracy of data entry.

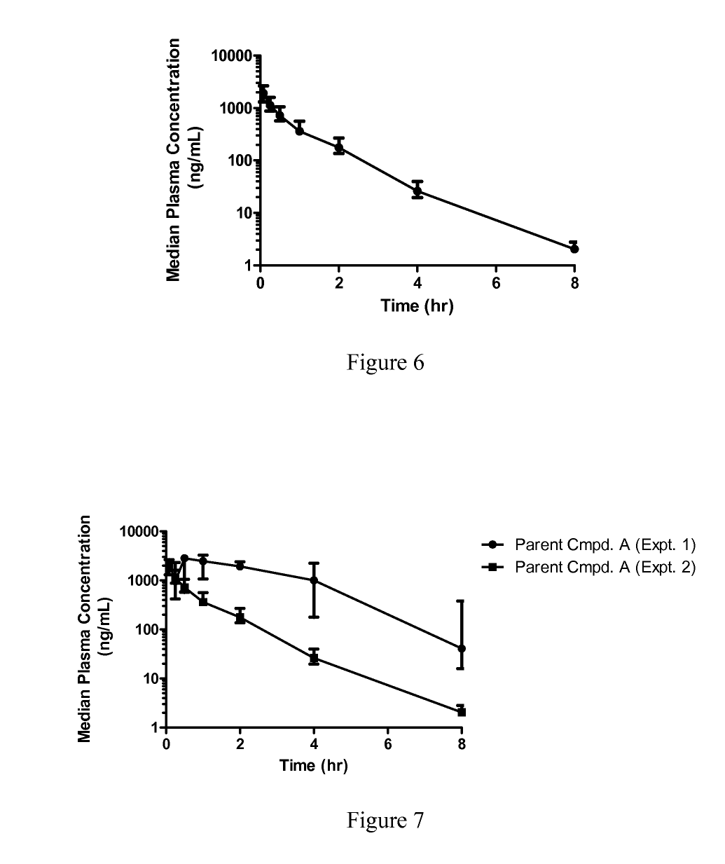

Pharmacokinetic parameters were estimated on median plasma concentrations using extravascular input (PO). Nominal collection times after administration of compound were used for PK analyses. The use of a non-compartmental model provided an adequate fit of the data. Pharmacokinetic parameters estimated for Parent Compound A included the maximum observed drug concentration (Cₘₐₓ), time of maximum observed drug concentration (Tₘₐₓ), apparent terminal half-life (t_{1/2}), apparent volume of distribution (Vz obs), apparent clearance (C1 obs), area under the concentration-time curve from time zero to 8 hours postdose of Example Compound 35 (AUC_{(0-inf)}), and area under the concentration-time curve extrapolated to infinity (AUC_{(0-inf)}). Example Compound 35 was detected in only one 0.25 hour hepatic portal vein sample at 1.29 ng/mL. Any Example Compound 35 present in all of the other samples was below the quantitation limit of 1 ng/mL and pharmacokinetic parameters were not determined for those samples. The median PK parameters estimated for Parent Compound A following portal vein sampling of the oral dose of Example Compound 35 or following femoral vein sampling of the intravenous dose of Parent Compound A are given in the Table 5 below. Individual and median concentration of Parent Compound A and Example Compound 35 in hepatic portal vein following oral administration of Example Compound 35 (20 mg/kg) to rats pursuant to Experiment 1 are shown in Table 6 below. Individual and median plasma concentration of Parent Compound A in femoral vein following IV administration of Parent Compound A (2.5 mg/kg) to rats pursuant to Experiment 2 are shown in Table 7 below. FIG. 5 depicts median concentration of Parent Compound A and Example Compound 35 in hepatic portal vein of rats following a single oral dose of Example Compound 35 (20 mg/kg). FIG. 6 depicts median plasma concentration of Parent Compound A following a single IV dose of Parent Compound A (2.5 mg/kg) to rats. FIG. 7 depicts a comparison of median concentration of Parent Compound A in hepatic portal vein samples following oral dose of Example Compound 35 at 20 mg/kg and femoral vein samples following an IV dose of Parent Compound A at 2.5 mg/kg.

TABLE 5

| Ex. | Cmpd. | | Blood | t_{1/2} | Tmax | Cmax | AUC₍₀₋₈₎ | AUC_{(0-inf)} | % F |
|---|---|---|---|---|---|---|---|---|---|
| No. | dosed | ROA | Sample | (hr) | (hr) | (ng/mL) | (hr*ng/mL) | (hr*ng/mL) | (AUC_{(0-inf)}) |
| 1 | Ex. 35 | Oral | Portal | 1.0 | 1.0 | 2,819 | 6,994 | 7,014 | 90.2 |
| | (20 mg/kg) | | Vein* | | | | | | |
| 2 | Parent | IV | Femoral | 1.0 | 0.07 | 1,924 | 1,459 | 1,463 | N/A |
| | Cmpd. A | | Vein* | | | | | | |
| | (2.5 mg/kg) | | | | | | | | |

*Analyte reported for both experiments here was Parent Compound A

Example Compound 35 is not significantly transported into the hepatic portal vein of female rats when administered orally at 20 mg/kg. Example Compound 35 is rapidly converted to the active compound Parent Compound A following oral administration. The amount of Parent Compound A recovered in the portal vein of female rats following oral administration of Example Compound 35 was 90.2% of what an IV dose of Parent Compound A would be.

TABLE 6

| | Nominal | | | Median |
|---|---|---|---|---|
| | Time | [Ex. Cmp. 35] | [Par. Cmp. A] | [Par. Cmp. A] |
| Animal ID | (hr) | (ng/mL) | (ng/mL) | (ng/mL) |
| 1116 | 0.25 | 0.38* | 417.4 | 961.4 |
| 1117 | 0.25 | 1.3 | 2,306.7 | |
| 1118 | 0.25 | 0.75* | 961.4 | |
| 1116 | 0.5 | 0.4* | 577.7 | 2818.9 |
| 1117 | 0.5 | 0.8* | 2,819.4 | |
| 1118 | 0.5 | 0.32* | 2,818.9 | |
| 1116 | 1 | 0.24* | 1,064.4 | 2455.1 |
| 1117 | 1 | 0.38* | 2,455.1 | |
| 1118 | 1 | 0.29* | 3,237.2 | |
| 1116 | 2 | 0.15* | 1,804.2 | 1930.2 |
| 1117 | 2 | 0.3* | 2,398.5 | |
| 1118 | 2 | No Peak | 1,930.2 | |
| 1116 | 4 | No Peak | 1,004.3 | 1004.3 |
| 1117 | 4 | 0.13* | 2,254.2 | |
| 1118 | 4 | 0.13* | 177.4 | |
| 1116 | 8 | No Peak | 41.0 | 41.0 |
| 1117 | 8 | 0.16* | 378.5 | |
| 1118 | 8 | 0.07* | 15.9 | |

*Below the quantitation limit of 1 ng/mL

TABLE 7

| | | | Median |
|---|---|---|---|
| | Nominal Time | [Par. Cmp. A] | [Par. Cmp. A] |
| Animal ID | (hr) | (ng/mL) | (ng/mL) |
| 10421 | 0.083 | 1,303.7 | 1924.0 |
| 10422 | 0.083 | 2,660.1 | |
| 10423 | 0.083 | 1,924.0 | |
| 10421 | 0.25 | 878.3 | 1131.8 |
| 10422 | 0.25 | 1,608.9 | |
| 10423 | 0.25 | 1,131.8 | |
| 10421 | 0.5 | 568.4 | 719.5 |
| 10422 | 0.5 | 1,062.6 | |
| 10423 | 0.5 | 719.5 | |
| 10421 | 1 | 343.1 | 360.4 |
| 10422 | 1 | 565.0 | |
| 10423 | 1 | 360.4 | |
| 10421 | 2 | 136.5 | 177.2 |
| 10422 | 2 | 268.6 | |
| 10423 | 2 | 177.2 | |
| 10421 | 4 | 19.7 | 26.2 |
| 10422 | 4 | 39.7 | |
| 10423 | 4 | 26.2 | |
| 10421 | 8 | 1.98* | 2.1 |
| 10422 | 8 | 2.05* | |
| 10423 | 8 | 2.81* | |

*Below the quantitation limit of 5 ng/mL

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be understood that certain changes and modifications can be practiced within the scope of the appended claims.

1. A compound having a structure according to Formula I 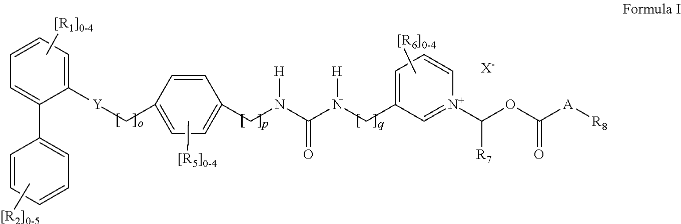
and pharmaceutically acceptable salts and solvates thereof;
wherein:
X is a pharmaceutically-acceptable counterion;
Y is —CH₂CH₂O—, —CH₂O—, —OCH₂—, —SCH₂—, —N(R)CH₂—, —N(R)C(═O)—, —C(═O)N(R)—, —S(═O)₂CH₂—, —S(═O)CH₂—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, ethylene, propylene, n-butylene, —O—C₁₋₄ alkylene-N(R)C(═O)—, —O—C₁₋₄ alkylene-C(═O)N(R)—, —N(R)C(═O)—C₁₋₄ alkylene-O—, —C(═O)N(R)—C₁₋₄ alkylene-O—, —C₁₋₄ alkylene-S(═O)₂—, —C₁₋₄ alkylene-S(═O)—, —S(═O)₂—C₁₋₄ alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —S—C₁₋₄ alkylene-, —C₁₋₄ alkylene-S—, —C₁₋₄ alkylene-S—C₁₋₄ alkylene-, —N(R)—C₁₋₄ alkylene-, —C₁₋₄ alkylene-N(R)—, —C₁₋₄ alkylene-N(R)—C₁₋₄ alkylene-, —C₁₋₄ alkylene-C(═O)—O—C₁₋₄ alkylene-, —C₁₋₄ alkylene-O—C(═O)—C₁₋₄ alkylene-, —C₁₋₄ alkylene-C(═O)—N(R)—N(R)—C₁₋₄ alkylene-, —C₁₋₄ alkylene-N(R)—C(═O)—C₁₋₄ alkylene-, —C(═O)—N(R)—C₁₋₄ alkylene-SO₂N(R)—, or —N(R)—C(═O)—C₁₋₄ alkylene-SO₂N(R)—;
R₁ and R₂, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heterocyclo, cycloalkyl, or amino;
R₅, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;
R₆, if present one or more times, is only attached to a ring carbon and is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;
A is optionally present and if present is selected from O, S, N(R₁₁), N(R₁₁)—C₁₋₄ alkylene, and C₁₋₄ alkylene;
R₁₁ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;
R₇ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;
R₈ is selected from optionally-substituted C₁₋₄ alkyl, optionally-substituted C₁₋₄ alkoxy, optionally-substituted C-carboxy, optionally-substituted aryl, optionally-substituted heteroaryl, optionally-substituted cycloalkyl, and optionally-substituted heterocyclyl;
o, p, and q are each independently 0, 1, or 2; and
any methylene group of the o, p, and q regions, Y, and A, are each optionally independently substituted with C₁₋₄ alkyl, halo, C₁₋₄ haloalkyl, or C₃ or C₄ cycloalkyl.

2. (canceled)

3. The compound according to claim 1, wherein Y is —S(═O)₂CH₂—, —S(═O)CH₂—, —CH₂O—, —CH₂CH₂O—, —CH₂S—, —CH₂N(R)—, —CH₂S(═O)₂—, —CH₂S(═O)—, —C(═O)O—, —OC(═O)—, —SO₂N(R)—, —N(R)SO₂—, —O—C₁₋₄ alkylene-N(R)C(═O)—, —C₁₋₄ alkylene-S(═O)₂—, —C₁₋₄ alkylene-S(═O)—, —S(═O)₂—C₁₋₄ alkylene-, —S(═O)—C₁₋₄ alkylene-, —C₁₋₄ alkylene-SO₂N(R)—, —C₁₋₄ alkylene-N(R)SO₂—, —SO₂N(R)—C₁₋₄ alkylene-, —N(R)SO₂—C₁₋₄ alkylene-, —C₁₋₄ alkylene-O—C₁₋₄ alkylene-, —O—C₁₋₄ alkylene-, —C₁₋₄ alkylene-O—, —C₁₋₄ alkylene-S—, —C₁₋₄ alkylene-S—C₁₋₄ alkylene-, —C₁₋₄ alkylene-N(R)—, —C₁₋₄ alkylene-N(R)—C₁₋₄ alkylene-, —C₁₋₄ alkylene-C(═O)—O—C₁₋₄ alkylene-, —C₁₋₄ alkylene-O—C(═O)—C₁₋₄ alkylene-, —C₁₋₄ alkylene-C(═O)—N(R)—C₁₋₄ alkylene-, or —C₁₋₄ alkylene-N(R)—C(═O)—C₁₋₄ alkylene-, wherein R is H, halo, C₁₋₅ alkyl, C₁₋₅ alkenyl, or C₁₋₅ alkynyl.

4. (canceled)

5. The compound according to claim 1, wherein said compound has a structure according to Formula Ia or Formula Ib: 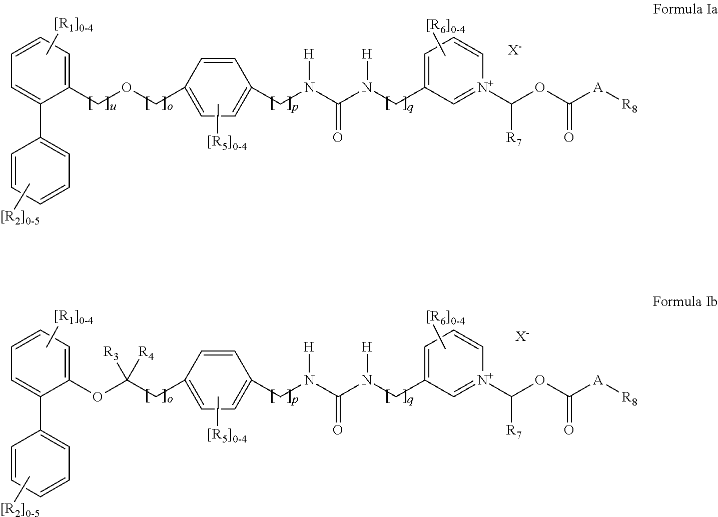
and pharmaceutically acceptable salts and solvates thereof;
wherein:
X is a pharmaceutically-acceptable counterion;
R₁ and R₂, if one or both are present one or more times, are each independently selected from halo, C₁₋₅ alkyl, nitro, cyano, C₁₋₅ alkoxy, C-amido, N-amido, trihalomethyl, C-carboxy, O-carboxy, sulfonamide, amino, aminoalkyl, hydroxyl, mercapto, alkylthio, sulfonyl, and sulfinyl, wherein C₁₋₅ alkyl, C₁₋₅ alkoxy, C-amido, N-amido, amino, aminoalkyl, and alkylthio are each optionally substituted with heterocyclo, cycloalkyl, or amino;
R₃ and R₄ are each independently H, halo, or C₁₋₄ alkyl, or R₃ and R₄ taken together form a cyclopropyl or cyclobutyl ring;
R₅, if present one or more times, is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;
R₆, if present one or more times, is only attached to a ring carbon and is independently selected from halo, C₁₋₅ alkyl, nitro, cyano, C₁₋₅ alkoxy, C-amido, N-amido, trihalomethyl, C-carboxy, O-carboxy, sulfonamide, amino, hydroxyl, mercapto, alkylthio, sulfonyl, and sulfinyl;
A is optionally present and if present is selected from O, S, N(R₁₁), N(R₁₁)—C₁₋₄ alkylene, and C₁₋₄ alkylene;
R₁₁ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;
R₇ is selected from hydro, C₁₋₆ alkyl, C₁₋₆ alkenyl, C₁₋₆ alkynyl, aryl, optionally-substituted aryl, optionally-substituted heteroaryl, optionally-substituted cycloalkyl, and optionally-substituted heterocyclyl;
R₈ is selected from optionally-substituted C₁₋₄ alkyl, optionally-substituted C₁₋₄ alkoxy, optionally-substituted C-carboxy, optionally-substituted aryl, optionally-substituted heteroaryl, optionally-substituted cycloalkyl, and optionally-substituted heterocyclyl;
o, p, and q are each independently 0, 1, or 2;
u is 1 or 2; and
any methylene group of the o, p, q, and u regions and A are each optionally independently substituted with C₁₋₄ alkyl, halo, C₁₋₄ haloalkyl, or C₃ or C₄ cycloalkyl.

6-11. (canceled)

12. The compound according to claim 1, wherein R₁ is not present, or when present is selected from the group consisting of halo, trihalomethyl, nitro, cyano, C-carboxy, O-carboxy, C-amido, N-amido C₁₋₅ alkyl, C₁₋₅ alkoxy, amino, aminoalkyl, and alkylthio, where applicable each further substituted with heterocyclo, cycloalkyl, or amino; or selected from the following: 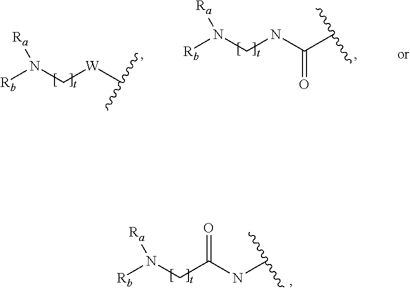
wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine.

13-14. (canceled)

15. The compound according to claim 1, wherein R₂ is not present, or when present is selected from C₁₋₅ alkyl, C₁₋₅ alkoxy, C-amido, N-amido, amino, aminoalkyl, or alkylthio, each further substituted with heterocyclo, cycloalkyl, or amino; or is selected from the following: 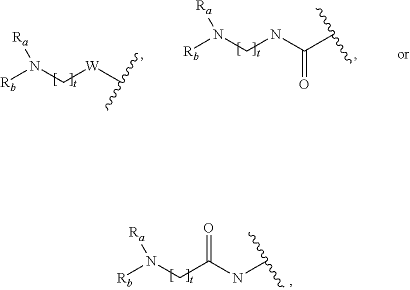
wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine.

16-17. (canceled)

18. The compound according to claim 1, wherein R₁ and/or R₂ is present and is located on the biphenyl ring as shown below: 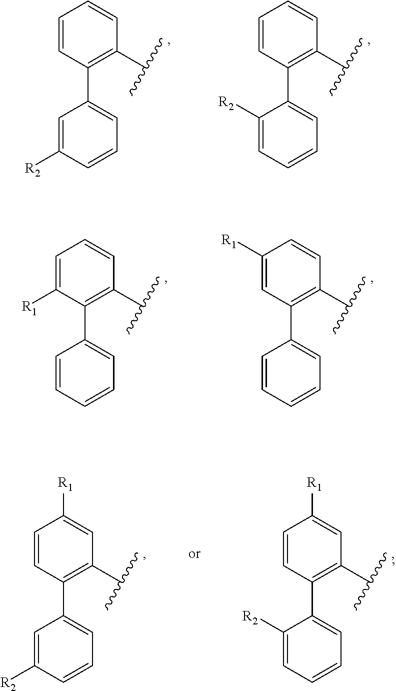
wherein R₁ and R₂ are each selected from the following: 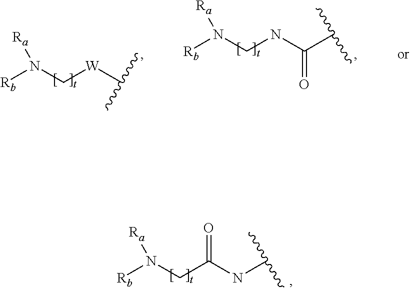
wherein t is 0, 1, 2, 3, or 4, W is N(H), O, C(H)₂, or S, and Rₐ and R_{b} are each independently hydro, C₃₋₆ cycloalkyl, or C₁₋₆ alkyl, or Rₐ and R_{b}, together with the linking nitrogen between them, form azetidine, pyrrolidine, or piperidine; with the proviso that when R₁ and R₂ are both present on the biphenyl ring, then R₁ is C₁₋₄ haloalkyl (such as, for example, trifluoromethyl) or halo (such as, for example, chloro).

19. (canceled)

20. The compound according to claim 1, wherein R₅, is not present or is fluoro, methyl, or trifluormethyl.

21-38. (canceled)

39. The compound according to claim 1, wherein R₁₁ and R₇ are each independently selected from hydro and C₁₋₄ alkyl.

40. (canceled)

41. The compound according to claim 1, wherein R₈ is selected from C₁₋₄ alkyl, C₁₋₄ alkoxy, methoxyethoxyethoxyethoxyethoxy, C-carboxy, aryl, heteroaryl, cycloalkyl, and heterocyclyl, wherein said aryl, heteroaryl, cycloalkyl, and heterocyclyl are each optionally-substituted with C-carboxy, O-carboxy, C₁₋₄ O-carboxyalkylene, hydroxyl, or hydroxylalkylene.

42. (canceled)

43. A compound selected from:
2-[2-[2-(2-methoxyethoxyl)ethoxy]ethoxy]ethyl[3-[[[4-[(2phenylphenyl)sulfonylamino]phenyl]carbamoylamino]methyl]-1-pyridyl]methyl carbonate;
1-[(Acetyloxy)methyl]-3-{[({4-[(biphenyl-2-yloxy)methyl]phenyl}carbamoyl)amino]methyl}pyridinium;
3-{[({4-[(Biphenyl-2-yloxy)methyl]phenyl}carbamoyl)amino]methyl}-1-{[(2,2-dimethylpropanoyl)oxy]methyl}pyridinium;
3-{[({4-[(Biphenyl-2-yloxy)methyl]phenyl}carbamoyl)amino]methyl}-1-(3-oxo-2,4,7,10,13,16-hexaoxaheptadec-1-yl) pyridinium;
3-{[({4-[(Biphenyl-2-yloxy)methyl]phenyl}carbamoyl)amino]methyl}-1-{[(methoxycarbonyl) oxy]methyl}pyridinium;
3-{[({4-[(Biphenyl-2-yloxy)methyl]phenyl}carbamoyl)amino]methyl}-1-[(5-methyl-2-oxo-1,3-dioxol-4-yl)methyl]pyridinium;
3-{[({4-[(Biphenyl-2-yloxy)methyl]phenyl}carbamoyl)amino]methyl}-1-[({[(2S)-2-(methoxycarbonyl)pyrrolidin-1-yl]carbonyl}oxy) methyl]pyridinium;
rel-3-{[({4-[(Biphenyl-2-yloxy)methyl]phenyl}carbamoyl)amino]methyl}-1-[({[(1R,2S)-2-(ethoxycarbonyl)cyclohexyl]carbamoyl}oxy) methyl]pyridinium;
rel-3-{[({4-[(Biphenyl-2-yloxy)methyl]phenyl}carbamoyl)amino]methyl}-1-[({[(1R,2R)-2-(ethoxycarbonyl)cyclohexyl]carbamoyl}oxy) methyl]pyridinium;
3-{[({4-[(Biphenyl-2-yloxy)methyl]phenyl}carbamoyl)amino]methyl}-1-[({[(2S)-2-(2,5,8,11,14-pentaoxapentadecan-1-oyl)pyrrolidin-1-yl]carbonyl}oxy) methyl]pyridinium;
3-{[({4-[(Biphenyl-2-yloxy)methyl]phenyl}carbamoyl)amino]methyl}-1-{1-[(2,2-dimethylpropanoyl)oxy]ethyl}pyridinium;
3-{[({4-[(Biphenyl-2-yloxy)methyl]phenyl}carbamoyl)amino]methyl}-1-{1-[(2,2-dimethylpropanoyl)oxy]-2-methylpropyl}pyridinium;
1-[(Benzoyloxy)methyl]-3-{[({4-[(biphenyl-2-yloxy)methyl]phenyl}carbamoyl)amino]methyl}pyridinium;
3-{[({4-[(Biphenyl-2-yloxy)methyl]phenyl}carbamoyl)amino]methyl}-1-{[(methylcarbamoyl)oxy]methyl}pyridinium;
3-{[({4-[(Biphenyl-2-yloxy)methyl]phenyl}carbamoyl)amino]methyl}-1-{[(dimethylcarbamoyl) oxy]methyl}pyridinium;
3-{[({4-[(Biphenyl-2-yloxy)methyl]phenyl}carbamoyl)amino]methyl}-1-{[(pyrrolidin-1-ylcarbonyl)oxy]methyl}pyridinium;
3-{[({4-[(Biphenyl-2-yloxy)methyl]phenyl}carbamoyl)amino]methyl}-1-{[(morpholin-4-ylcarbonyl)oxy]methyl}pyridinium;
Methyl N-{[(3-{[({4-[(biphenyl-2-yloxy)methyl]phenyl}carbamoyl)amino]methyl}pyridinium-1-yl)methoxy]carbonyl}-N-methylglycinate;
Methyl N-{[(3-{[({4-[(biphenyl-2-yloxy)methyl]phenyl}carbamoyl)amino]methyl}pyridinium-1-yl)methoxy]carbonyl}-beta-alaninate;
3-{[({4-[(Biphenyl-2-yloxy)methyl]phenyl}carbamoyl)amino]methyl}-1-[({[(2S)-2-(methoxycarbonyl)piperidin-1-yl]carbonyl}oxy) methyl]pyridinium;
3-{[({4-[(Biphenyl-2-yloxy)methyl]phenyl}carbamoyl)amino]methyl}-1-({[(2-methoxyethyl) carbamoyl]oxy}methyl)pyridinium;
3-{[({4-[(Biphenyl-2-yloxy)methyl]phenyl}carbamoyl)amino]methyl}-1-{1-[(ethoxycarbonyl) oxy]ethyl}pyridinium;
3-{[({4-[(Biphenyl-2-yloxy)methyl]phenyl}carbamoyl)amino]methyl}-1-[1-({[(2S)-2-(methoxycarbonyl)pyrrolidin-1-yl]carbonyl}oxy) ethyl]pyridinium
1-[1-(Acetyloxy)ethyl]-3-{[({4-[(biphenyl-2-yloxy)methyl]phenyl}carbamoyl)amino]methyl}pyridinium;
3-{[({4-[(Biphenyl-2-yloxy)methyl]phenyl}carbamoyl)amino]methyl}-1-[(butanoyloxy) methyl]pyridinium;
1-{[({(2S)-2-[(Acetyloxy)methyl]pyrrolidin-1-yl}carbonyl)oxy]methyl}-3-{[({4-[(biphenyl-2-yloxy)methyl]phenyl}carbamoyl)amino]methyl}pyridinium;
1-[1-(Acetyloxy)-2-methylpropyl]-3-{[({4-[(biphenyl-2-yloxy)methyl]phenyl}carbamoyl) amino]methyl}pyridinium;
3-{[({4-[(Biphenyl-2-yloxy)methyl]phenyl}carbamoyl)amino]methyl}-1-{1-[(2-methylpropanoyl)oxy]ethyl}pyridinium;
3-{[({4-[(Biphenyl-2-yloxy)methyl]phenyl}carbamoyl)amino]methyl}-1-{[(2-methylpropanoyl) oxy]methyl}pyridinium;
Methyl N-{[(3-{[({4-[(biphenyl-2-yloxy)methyl]phenyl}carbamoyl)amino]methyl}pyridinium-1-yl)methoxy]carbonyl}glycinate;
3-{[({4-[(Biphenyl-2-yloxy)methyl]phenyl}carbamoyl)amino]methyl}-1-[({[2-(methoxycarbonyl)piperidin-1-yl]carbonyl}oxy) methyl]pyridinium;
1-[1-(Benzoyloxy)ethyl]-3-{[({4-[(biphenyl-2-yloxy)methyl]phenyl}carbamoyl)amino]methyl}pyridinium;
1-[1-(Benzoyloxy)-2-methylpropyl]-3-{[({4-[(biphenyl-2-yloxy)methyl]phenyl}carbamoyl) amino]methyl}pyridinium;
1-[(Acetyloxy)methyl]-3-{[({4-[2-(biphenyl-2-yl)ethoxy]phenyl}carbamoyl)amino]methyl}pyridinium;
1-[(Benzoyloxy)methyl]-3-{[({4-[2-(biphenyl-2-yl)ethoxy]phenyl}carbamoyl)amino]methyl}pyridinium;
3-{[({4-[2-(Biphenyl-2-yl)ethoxy]phenyl}carbamoyl)amino]methyl}-1-{[(2,2-dimethylpropanoyl)oxy]methyl}pyridinium;
1-[1-(Acetyloxy)ethyl]-3-{[({4-[2-(biphenyl-2-yl)ethoxy]phenyl}carbamoyl)amino]methyl}pyridinium;
1-[1-(Acetyloxy)-2-methylpropyl]-3-{[({4-[2-(biphenyl-2-yl)ethoxy]phenyl}carbamoyl) amino]methyl}pyridinium;
3-{[({4-[2-(Biphenyl-2-yl)ethoxy]phenyl}carbamoyl)amino]methyl}-1-{1-[(2,2-dimethylpropanoyl)oxy]ethyl}pyridinium;
3-{[({4-[2-(Biphenyl-2-yl)ethoxy]phenyl}carbamoyl)amino]methyl}-1-{1-[(2,2-dimethylpropanoyl)oxy]-2-methylpropyl}pyridinium;
3-{[({4-[2-(Biphenyl-2-yl)ethoxy]phenyl}carbamoyl)amino]methyl}-1-[(butanoyloxy)methyl]pyridinium;
1-[1-(Benzoyloxy)ethyl]-3-{[({4-[2-(biphenyl-2-yl)ethoxy]phenyl}carbamoyl)amino]methyl}pyridinium;
1-[1-(Benzoyloxy)-2-methylpropyl]-3-{[({4-[2-(biphenyl-2-yl)ethoxy]phenyl}carbamoyl) amino]methyl}pyridinium;
3-{[({4-[2-(Biphenyl-2-yl)ethoxy]phenyl}carbamoyl)amino]methyl}-1-{[(2-methylpropanoyl)oxy]methyl}pyridinium;
1-{[({(2S)-2-[(Acetyloxy)methyl]pyrrolidin-1-yl}carbonyl)oxy]methyl}-3-{[({4-[2-(biphenyl-2-yl)ethoxy]phenyl}carbamoyl)amino]methyl}pyridinium;
3-{[({4-[2-(Biphenyl-2-yl)ethoxy]phenyl}carbamoyl)amino]methyl}-1-[({[(2S)-2-(hydroxymethyl)pyrrolidin-1-yl]carbonyl}oxy) methyl]pyridinium;
3-{[({4-[2-(Biphenyl-2-yl)ethoxy]phenyl}carbamoyl)amino]methyl}-1-{1-[(2-methylpropanoyl) oxy]ethyl}pyridinium;
Methyl N-{[(3-{[({4-[2-(biphenyl-2-yl)ethoxy]phenyl}carbamoyl)amino]methyl}pyridinium-1-yl)methoxy]carbonyl}glycinate;
Methyl N-{[(3-{[({4-[2-(biphenyl-2-yl)ethoxy]phenyl}carbamoyl)amino]methyl}pyridinium-1-yl)methoxy]carbonyl}-beta-alaninate;
Methyl N-{[(3-{[({4-[2-(biphenyl-2-yl)ethoxy]phenyl}carbamoyl)amino]methyl}pyridinium-1-yl)methoxy]carbonyl}-N-methylglycinate;
3-{[({4-[2-(Biphenyl-2-yl)ethoxy]phenyl}carbamoyl)amino]methyl}-1-(methoxymethyl) pyridinium;
3-{[({4-[2-(Biphenyl-2-yl)ethoxy]phenyl}carbamoyl)amino]methyl}-1-[({[(2S)-2-(methoxycarbonyl)pyrrolidin-1-yl]carbonyl}oxy) methyl]pyridinium;
3-{[({4-[2-(Biphenyl-2-yl)ethoxy]phenyl}carbamoyl)amino]methyl}-1-{[(pyridin-3-ylcarbonyl)oxy]methyl}pyridinium;
3-{[({4-[2-(Biphenyl-2-yl)ethoxy]phenyl}carbamoyl)amino]methyl}-1-{[(4-methoxy-4-oxobutanoyl)oxy]methyl}pyridinium;
3-{[({4-[2-(Biphenyl-2-yl)ethoxy]phenyl}carbamoyl)amino]methyl}-1-{[(2-hydroxybenzoyl) oxy]methyl}pyridinium;
3-{[({4-[2-(Biphenyl-2-yl)ethoxy]phenyl}carbamoyl)amino]methyl}-1-{[(diethylcarbamoyl)oxy]methyl}pyridinium;
3-{[({4-[2-(Biphenyl-2-yl)ethoxy]phenyl}carbamoyl)amino]methyl}-1-[({[(2S)-2-(2-hydroxypropan-2-yl)pyrrolidin-1-yl]carbonyl}oxy) methyl]pyridinium;
3-{[({4-[2-(Biphenyl-2-yl)ethoxy]phenyl}carbamoyl)amino]methyl}-1-({[(2-methoxyethyl) carbamoyl]oxy}methyl)pyridinium;
3-{[({4-[2-(Biphenyl-2-yl)ethoxy]phenyl}carbamoyl)amino]methyl}-1-{[(pyrrolidin-1-ylcarbonyl)oxy]methyl}pyridinium; and
3-{[({4-[2-(Biphenyl-2-yl)ethoxy]phenyl}carbamoyl)amino]methyl}-1-{[(morpholin-4-ylcarbonyl)oxy]methyl}pyridinium;
and pharmaceutically acceptable salts and solvates thereof.

44. A pharmaceutical composition comprising a compound of claim 1 and a pharmaceutically acceptable excipient.

45. A method of treating cancer, comprising administering a therapeutically effective amount of a compound of claim 1 or a pharmaceutical composition thereof to a patient.

46-47. (canceled)

48. The method of claim 45, further comprising administering to said patient a therapeutically effective amount of a second agent selected from the group consisting of PARP activators, PARP inhibitors, non-DNA damaging agents which are not PARP activators, DNA-damaging agents, and thymidylate synthase inhibitors.

49. (canceled)

50. The method of claim 48, wherein said PARP activator is selected from alkylating agents, methyl methane sulfonate (MMS), N-methyl-N′nitro-N-nitrosoguanidine (MNNG), Nitrosoureas, N-methyl-N-nitrosourea (MNU), streptozotocin, carmustine, lomustine, Nitrogen mustards, melphalan, cyclophosphamide, uramustine, ifosfamide, clorambucil, mechlorethamine, alkyl sulfonates, busulfan, platins, cisplatin, oxaliplatin, carboplatin, nedaplatin, satraplatin, triplatin tetranitrate, non-classical DNA alkylating agents, temozolomide, dacarbazine, mitozolamide, procarbazine, altretamine, radiation, X-rays, gamma rays, charged particles, UV, systemic or targeted radioisotope therapy, DNA damaging agents, topoisomerase inhibitors, camptothecin, beta-lapachone, irinotecan, etoposide, anthracyclines, doxorubicin, daunorubicin, epirubicin, idarubicin, valrubicin, mitoxantrone, reactive oxygen generators, menadione, peroxynitrite, and anti-metabolites, 5-FU, raltetrexed, pemetrexed, pralatrexate, methotrexate, gemcitabine, thioguanine, fludarabine, azathioprine, cytosine arabinoside, mercaptopurine, pentostatin, cladribine, folic acid, and floxuridine; said PARP inhibitor is selected from olaparib, AG014699/PF-01367338, INO-1001, ABT-888, Iniparib, BSI-410, CEP-9722, MK4827, and E7016; said DNA damaging agent is selected from DNA damaging agents, topoisomerase inhibitors, camptothecin, beta-lapachone, irinotecan, etoposide, anthracyclines, doxorubicin, daunorubicin, epirubicin, idarubicin, valrubicin, mitoxantrone, reactive oxygen generators, menadione, peroxynitrite, and anti-metabolites, 5-FU, raltetrexed, pemetrexed, pralatrexate, methotrexate, gemcitabine, thiouanine, fludarabine, azathioprine, cytosine arabinoside, mercaptopurine, pentostatin, cladribine, folic acid, and floxuridine; and said thymidylate synthase inhibitor is selected from 5-FU, raltitrexed, and pemetrexed.

51-62. (canceled)

63. The method of claim 45, wherein cells of said cancer exhibit low levels of Naprt1 expression.

64. The method of claim 63, further comprising administering nicotinic acid, or a compound capable of forming nicotinic acid in vivo, to said patient.

65-66. (canceled)

67. The method of claim 45, wherein said cancer is selected from the group consisting of Hodgkin's disease, non-Hodgkin's lymphoma, acute lymphocytic leukemia, chronic lymphocytic leukemia, acute myeloid leukemia, mantle-cell lymphoma, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer, and prostatic carcinoma.

68. (canceled)

69. A method of treating, delaying the onset, or reducing the severity of, one or more symptoms of cancer, systemic or chronic inflammation, rheumatoid arthritis, diabetes, obesity, T-cell mediated autoimmune disease, ischemia, and other complications associated with these diseases and disorders, in a human patient, comprising identifying a patient in need of such treatment and administering a therapeutically effective amount of a compound of claim 1 or a pharmaceutical composition thereof.

70-75. (canceled)

76. A method of identifying a cancer that is likely susceptible to treatment with a compound of claim 1, said method comprising:
obtaining a biopsy sample of said cancer;
determining the expression level of enzymes in pathways for NAD biosynthesis relative to a non-cancerous control tissue, wherein,
if the expression level of enzymes in such pathways is reduced relative to a non-cancerous control tissue, the cancer is identified as likely susceptible to treatment with a compound of claim 1.

77. A method of making a compound, comprising:
reacting a compound having a structure according to Parent Compound I, wherein R₁, R₂, R₅, R₆, Y, o, p, and q are as defined for Formula I in claim 1; 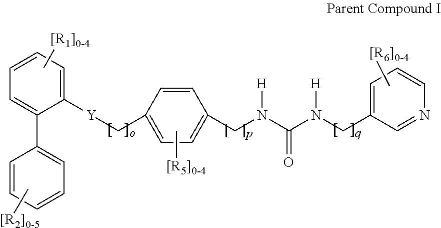
with a desired Prodrug Moiety, wherein X is Cl or Br and A, R₇, and R₈ are as defined for Formula I in claim 1; 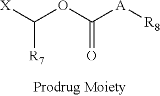
under suitable conditions to yield a compound having a structure according to Formula I as defined in claim 1.
